SEQUENCE LISTING (153 sequences)

SEQ ID NO: 1 - protein, 456 aa, Arabidopsis thaliana
MGCFSCFDSSDDEKLNPVDESNHGQKKQSQPTVSNNISGLPSGGEKLSSKTNGGSKRELLLPRDGLGQIAAHTFAFRELAAATMNFHPDTFLGEGGFGRVYKGRLDSTGQVVAVKQLDRNGLQGNREFLVEVLMLSLLHHPNLVNLIGYCADGDQRLLVYEFMPLGSLEDHLHDLPPDKEALDWNMRMKIAAGAAKGLEFLHDKANPPVIYRDFKSSNILLDEGFHPKLSDFGLAKLGPTGDKSHVSTRVMGTYGYCAPEYAMTGQLTVKSDVYSFGVVFLELITGRKAIDSEMPHGEQNLVAWARPLFNDRRKFIKLADPRLKGRFPTRALYQALAVASMCIQEQAATRPLIADVVTALSYLANQAYDPSKDDSRRNRDERGARLITRNDDGGGSGSKFDLEGSEKEDSPRETARILNRDINRERAVAEAKMWGESLREKRRQSEQGTSESNSTG

SEQ ID NO: 2 - DNA, 1400 bp, Arabidopsis thaliana
atgggttgtttctcgtgttttgattcgagtgatgacgagaagctgaatccagttgatgaatctaatcatggtcagaagaaacaatcacaaccgacagtatccaataacatatctggactcccttcaggtggggagaagcttagctcaaagaccaatggaggatcaaaaagggagctactgcttccaagggatggacttggacaaattgctgctcatacatttgctttccgcgagcttgctgctgcaactatgaactttcatcctgacactttcttaggcgaaggtggatttggacgtgtctacaaaggaaggcttgacagcaccggtcaggttgttgctgttaaacaactagacaggaatggtctacaaggtaacagagaatttctggtagaggttcttatgctcagtcttcttcatcatcccaacttagtcaaccttattggttattgtgctgatggagatcaacgcctcttggtctacgagtttatgccgttaggatcattggaagatcacctccacgatcttccaccggataaggaggccttagattggaacatgagaatgaaaatagctgctggtgcggcgaaaggattggaatttctacatgataaggcaaaccctccggttatttatagagattttaagtcatcaaatattttactggatgagggtttccaccctaagctttctgattttggacttgctaaactcggaccaacgggagacaaatctcatgtctccactagagttatgggaacttatggttattgtgctcccgagtacgcaatgacgggacaattgacagtaaaatcagatgtctacagttttggtgtggtttttctcgagctgattactggtcgcaaagctatagacagcgagatgcctcatggagagcagaacctggtggcttgggctcgcccattgttcaacgacaggcgaaagttcataaaactggctgatccaaggttaaaggggcggtttccaacgcgtgcactctaccaagctttagctgtggcatcaatgtgcatccaagaacaggcggctacacgtcctctcatagcagatgttgtcactgcactctcctatcttgcaaaccaagcttatgatccaagtaaagatgatagtagaagaaaccgggatgaaagaggtgcaaggttaataacaaggaacgacgatggaggtggctcgggaagtaaattcgatttagaaggttcagagaaagaagattcaccgagagagacagctcggatattgaaccgagatatcaatagggagcgtgcggttgcagaggctaagatgtggggagagagtttgagggagaaacgaagacagagcgagcagggtacttcagagagcaacagtaccggggattacaaggatgacgacgataagtaggagct

SEQ ID NO: 3 - protein, 889 aa, Arabidopsis thaliana
MGGCFSVSLPCDQVVSQFSQLLCVRGSYIHNLSKNLASLQKAMRMLKARQYDVIRRLETEEFTGRQQRLSQVQVWLTSVLIIQNQFNDLLRSNEVELQRLCLCGFCSKDLKLSYRYGKRVIMMLKEVESLSSQGFFDVVSEATPFADVDEIPFQPTIVGQEIMLEKAWNRLMEDGSGILGLYGMGGVGKTTLLTKINNKFSKIDDRFDVVIWVVVSRSSTVRKIQRDIAEKVGLGGMEWSEKNDNQIAVDIHNVLRRRKFVLLLDDIWEKVNLKAVGVPYPSKDNGCKVAFTTRSRDVCGRMGVDDPMEVSCLQPEESWDLFQMKVGKNTLGSHPDIPGLARKVARKCRGLPLALNVIGEAMACKRTVHEWCHAIDVLTSSAIDFSGMEDEILHVLKYSYDNLNGELMKSCFLYCSLFPEDYLIDKEGLVDYWISEGFINEKEGRERNINQGYEIIGTLVRACLLLEEERNKSNVKMHDVVREMALWISSDLGKQKEKCIVRAGVGLREVPKVKDWNTVRKISLMNNEIEEIFDSHECAALTTLFLQKNDVVKISAEFFRCMPHLVVLDLSENQSLNELPEEISELASLRYFNLSYTCIHQLPVGLWTLKKLIHLNLEHMSSLGSILGISNLWNLRTLGLRDSRLLLDMSLVKELQLLEHLEVITLDISSSLVAEPLLCSQRLVECIKEVDFKYLKEESVRVLTLPTMGNLRKLGIKRCGMREIKIERTTSSSSRNKSPTTPCFSNLSRVFIAKCHGLKDLTWLLFAPNLTFLEVGFSKEVEDIISEEKAEEHSATIVPFRKLETLHLFELRGLKRIYAKALHFPCLKVIHVEKCEKLRKLPLDSKSGIAGEELVIYYGEREWIERVEWEDQATQLRFLPSSRWRWRET

SEQ ID NO: 4 - DNA, 2670 bp, Arabidopsis thaliana
atgggaggttgtttctctgtttcattgccatgtgatcaagtagtgagtcagttctctcagttgttatgtgtcaggggaagttatattcacaatctctccaagaatctggcttctctgcagaaggccatgcgaatgctcaaggctcggcaatatgatgtgataagaaggttggaaacagaagagtttacagggcgtcaacaaaggctttcccaagttcaggtatggcttactagtgtcctaatcattcaaaaccagtttaatgatctgcttcgtagtaatgaagttgagcttcaaaggttgtgtctttgtggtttctgctccaaagatttgaaattgagctatcgttatgggaaaagagttatcatgatgttgaaggaagttgagagtctcagttctcaaggattctttgatgtggtttctgaggcaactccgtttgctgatgtggatgagatcccttttcaacccacaattgttggtcaggagataatgcttgaaaaggcatggaaccgtctcatggaagatggttccgggattttgggtctgtacggtatggggggagtaggcaaaacgacactactcacgaagatcaacaataagttttctaaaatagatgacagatttgatgttgtgatatgggttgttgtgtctagaagttcaacagtccgtaagatacaaagagacatagcagaaaaggtaggccttgggggaatggagtggagcgagaaaaacgataaccagatagccgttgacatccacaatgtccttaggagacgaaagtttgttttattgttggatgacatatgggagaaagtgaatttaaaagcggttggagtcccgtatccaagcaaagataacggatgcaaggtagcattcactactcgttctcgagatgtgtgtgggcgcatgggggttgatgatccgatggaagttagctgtctacaacccgaagaatcttgggatttgtttcaaatgaaggttgggaaaaatacactgggaagtcacccagacattcccggacttgctagaaaagtcgctaggaaatgtcgtggcctaccattagcactcaatgttatcggtgaagctatggcatgcaaaagaacagtacatgaatggtgtcatgccattgacgttttaacatcatctgccatagatttttcaggtatggaagatgaaattctccatgttttgaagtatagctatgataatttaaatggggagctgatgaaatcctgcttcctctattgctctctgtttcctgaagactatcttattgataaagaggggttggtagactactggataagtgagggattcataaatgaaaaagaaggcagagagaggaatattaaccaaggttatgagataattggtacccttgtccgtgcatgtttgttgttggaggaagaaaggaacaaatcaaacgtgaaaatgcatgatgtggttcgtgagatggctctatggatatcatccgatcttgggaagcaaaaggagaaatgtattgtgcgagctggtgttgggttacgtgaagtaccaaaagtcaaggattggaacactgtgagaaagatttctttgatgaataatgagattgaagagatatttgacagtcacgagtgtgctgcacttacaactctatttctccaaaagaacgacgtggtaaagatctcggctgaattctttcgatgtatgcctcatctggtggttttagatctatcagagaatcagagtcttaatgaattaccagaagaaatatcagagctggcctctttgagatatttcaacttgtcatatacatgtatacatcaactacccgttggtttatggacattgaaaaagctaatacatctgaatctcgaacacatgagcagtcttgggagtatattagggatatcaaatttgtggaatttgaggacattgggactacgagattccagactgttgctagatatgagtttagtgaaggagctgcagctcttagaacatctagaagttataaccctagatatatcatcaagtttggttgcagagccattgttatgctctcaaaggttggtggaatgtataaaagaggtagattttaagtaccttaaggaagaatcagtgagagtattgactttgccaaccatgggtaatctccgcaagctcgggataaaaagatgtggaatgagggagataaagatagagaggacaacttcatcatcgtctcggaacaaaagtcccacaactccatgcttctcaaacctctccagagtctttatagctaaatgccatggtcttaaggatttgacgtggcttttgtttgctcctaatcttacttttcttgaagttgggttctcaaaggaagtagaggatataattagtgaagagaaagctgaggagcattcagctacgattgttccttttaggaaattggagacactacacctgtttgaattacgcgggctcaagagaatctatgcaaaggctctgcattttccgtgtctgaaagttatccacgtagaaaaatgtgaaaagctgagaaagcttccattggattctaaaagtgggatcgcgggtgaagaacttgtcatatattatggagagagagaatggatagaaagggttgaatgggaggaccaagcaactcaactccgtttcttaccttcatccaggtggcggtggagagaaacataa

SEQ ID NO: 5 - DNA, 1949 bp, Artificial Sequence
  Promoter
gtgattgcggttacatcatgtacggaaaaataattctaatccttgatttaaatttgatcttgactatttatttattctttatttcattttgtaaatcattttatgtatcccctggcaaggcaattttatccaccttgcacccaacacttcgggttcccataaatcaaaccaccttaacttcacacaatgctgtaactcacaccgcccagcatctccaatgtgaaagaagctaaaatttaataaacaatcatacgaagcagtgacaaaataccagatggtattaatgctttgataaaattaattggaaagtataaaatggtaggaaataattaattataattaatttaaataagattaaaaataatttaaaactaaaatgttaaaattttaagaaaattattttaaataatatttaaaaacatttaaaatcattttaaaaaatttatttataggacaattaaatgaatatttcagctaattaaaaacaaaagcttacctagccttagaagacaacttgtccaacaattagatgatacccattgcccttacgttttctttaacatcaattattgtttttgtcaacaagctatcttttagttttattttattggtaaaaaatattgtcgccttcaagttgcatcatttaacacatctcgtcattagaaaaataaaactcttccctaaacgattagtagaaaaaatcattcgataataaataagcaagcaaaattaggaaaaaataacttcattttaaaaaaatcattaaggctatattttttaaatgactaattttatatagactgtaactaaaagtatacaatttattatgctatgtatcttaaagaattacttataaaaatctacggaagaatatcttacaaagtgaaaaacaaatgagaaagaatttagtgggatgattatgattttatttgaaaattgaaaaaataattattaaagactttagtggagtaagaaagctttcctattagtcttttcttatccataaaaaaaaaaaaaaaaatctagcgtgacagcttttccatagattttaataatgtaaaatactggtagcagccgaccgttcaggtaatggacactgtggtcctaacttgcaacgggtgcgggcccaatttaataacgccgtggtaacggataaagccaagcgtgaaggggtgaaggtacatctctgactccgtcaagattacgaaaccgtcaactacgaaggactccccgaaatatcatctgtgtcataaacaccaagtcacaccatacatgggcacgcgtcacaatatgattggagaacggttccaccgcatatgctataaattgcccccacacccctcgaccctaatcgcacttgaattgcaatcaaattagttcattctctttgcgcagttccctacctccccctttcaaggtccgtagattcctcctgtttttttttcttcttctttattgtttgttctacatcagcatgatgttgatttgattgtgttttctatcgtttcatcgattataaattttcataatcagaagattcagcttttattaatgcaagaacgtccttaattgatgattttataaccgtaaattaggtctaattagagtttttttcataaagattttcagatccgtttacaacaagccttaattgttgattctgtagtcgtagattaaggtttttttcatgaactacttcagatccgttaaacaacagccttatttgttgatacttcagtcgtttttcaagaaattgttcagatccgttgataaaagccttattcgttgattctgtatggtatttcaagagatattgctcaggtcctttagcaactaccttatttgttgattctgtggccatagattaggattttttttcacgaaattgcttcttgaaattacgtgatggattttgattctgatttatcttgtgattgttgactctacag

SEQ ID NO: 6 - DNA, 2002 bp, Artificial Sequence
  Promoter
ccttaggaaatatggtggctgtactcattttgatgttgatgaatggtttaactgaattccgtttattatcatttttttttttttttgtattatataaaatgaaatgtatagagttgtacaaaaaaaatgaaaccagagtttagagtttatcaacatagagccaaaagttcacgcgataggggtcaaaatcgaaatatagtgtgaacaaataataaaacataatgaaataaagcaaaatcataaaagaaaaagataaaataatgtttgtacaaaaaaaaaagataaaataatgattgtagtatctaccaaaaaaataataataataatgatggtagtctctcatctgtttgactgaattaaatttatttgacaactttaaatgagttttttctttgataaaaactttaaattatagtataaataatatattataatacctatacacatatatttaaaaagttaagaaaatgggtagaccatgtcatggtttaaaaataaaaaataatttgacaaaaatcataaaaaattaaataaccaagttaataataacaagaatatttaatcatgattttattatattgttgttgacctattgactaatacatactaaaaataaacacacaagaaggagtatggagacctaaaatcgaactcaggattcaacataaaattgttatagtcaaaccattgggttataacaatggggacaataatgaaaatataaaaatgaaccaaaattataaaacaaaaaagataaaataatgatgacaatttctcatttgtttgattggtttggttttatttgacaattttaaatgagttaaataatatttctaatacatacatattaaaaatttaagaaaattgggtagaccatagatccttccgctttatatcaatataattttctcgatgtaaatgggcattttccctcttttgcacaaaaaaaaaaatctcgatgttacattgatatttatgtgcgcctaagactttacattgatatatttatttggtgcgccttcattttttctatattatatcataaattttaaaaacaaagttacaaacacaaagcgcgccttcatttttctatattatatcataaattagaattacgttctcgaagagtcaaaccaacctactgattacgttctcggagagacaaaccaacctagtgatttactactagctaattttggttattcctcgaaagtttactttataatcgtgaaatgttattctgattttcttttgaggccaaaatcgtatccgactcacgagagattgctagtttttcttttgcagccaaattcctttacattttaacaaaagaattttttttttttggtcaagtagccacctagtggctagagcacacacaatttaattgtggagaagtggagtgtctggggttcgaaccccggcccctgcatataatatgcaatatccctaccaactgagctaagctcacgagaattaaaaaaaaagaatattaatctaactaaatatacagttaagcgcaatagaaaaacagacgaacaaaagaaagaaacgttgatatataattattggagaatgctaaccattgttagtattgaaaaatggtgaaaaatgataaatttaactttttaaaagtcaaaatattattgaaatcaatataaacatactacttttgacttctttaaccattaccagagggcaatggttaacgagacccataattattctaaggagctactttgcagataggggatgtccttttttttttttgttgaaagaaattaggggatgtccttatctctttatccctgccaattttcctatttataaataaatatcgagtttatcgctctccacctcaaacgctttctttcttgttcttggtttagggttttcttcacccattagggttagggttcaattttccccttttcaatccccaaatcaaaattcaatcaaaacctcaaatttctcaccttttctcactcaatcatcaatcaag

SEQ ID NO: 7 - protein, 7 aa, Arabidopsis thaliana
GDKSHVS

SEQ ID NO: 8 - protein, 6 aa, Artificial Sequence
  Cleavage peptide
RWWFAL

SEQ ID NO: 9 - protein, 6 aa, Artificial Sequence
  Cleavage peptide
HWVNFL

SEQ ID NO: 10 - protein, 6 aa, Artificial Sequence
  Cleavage peptide
WAELVL

SEQ ID NO: 11 - protein, 6 aa, Artificial Sequence
  Cleavage peptide
PIISLA

SEQ ID NO: 12 - protein, 6 aa, Artificial Sequence
  Cleavage peptide
WFYVLQ

SEQ ID NO: 13 - protein, 6 aa, Artificial Sequence
  Cleavage peptide
LTEMFM

SEQ ID NO: 14 - protein, 6 aa, Artificial Sequence
  Cleavage peptide
GQYFVW

SEQ ID NO: 15 - protein, 6 aa, Artificial Sequence
  Cleavage peptide
YWTTLF

SEQ ID NO: 16 - protein, 6 aa, Artificial Sequence
  Cleavage peptide
IQMLWA

SEQ ID NO: 17 - protein, 6 aa, Artificial Sequence
  Cleavage peptide
YARFYL

SEQ ID NO: 18 - protein, 6 aa, Artificial Sequence
  Cleavage peptide
LAKLWY

SEQ ID NO: 19 - protein, 6 aa, Artificial Sequence
  Cleavage peptide
YFWLVR

SEQ ID NO: 20 - protein, 6 aa, Artificial Sequence
  Cleavage peptide
GFWLSF

SEQ ID NO: 21 - protein, 6 aa, Artificial Sequence
  Cleavage peptide
TLEEWF

SEQ ID NO: 22 - protein, 6 aa, Artificial Sequence
  Cleavage peptide
QQLFVV

SEQ ID NO: 23 - protein, 6 aa, Artificial Sequence
  Cleavage peptide
MRFYFT

SEQ ID NO: 24 - protein, 6 aa, Artificial Sequence
  Cleavage peptide
QVFEFL

SEQ ID NO: 25 - protein, 6 aa, Artificial Sequence
  Cleavage peptide
VYYFYR

SEQ ID NO: 26 - protein, 6 aa, Artificial Sequence
  Cleavage peptide
QMIFLR

SEQ ID NO: 27 - protein, 6 aa, Artificial Sequence
  Cleavage peptide
IFLWSA

SEQ ID NO: 28 - protein, 6 aa, Artificial Sequence
  Cleavage peptide
NLEFLY

SEQ ID NO: 29 - protein, 6 aa, Artificial Sequence
  Cleavage peptide
TFTFFQ

SEQ ID NO: 30 - protein, 6 aa, Artificial Sequence
  Cleavage peptide
TAMRIH

SEQ ID NO: 31 - protein, 6 aa, Artificial Sequence
  Cleavage peptide
LIMQKN

SEQ ID NO: 32 - protein, 6 aa, Artificial Sequence
  Cleavage peptide
VMLMHA

SEQ ID NO: 33 - protein, 6 aa, Artificial Sequence
  Cleavage peptide
VKSFLM

SEQ ID NO: 34 - protein, 6 aa, Artificial Sequence
  Cleavage peptide
HLYLYH

SEQ ID NO: 35 - protein, 6 aa, Artificial Sequence
  Cleavage peptide
LMFKMP

SEQ ID NO: 36 - protein, 6 aa, Artificial Sequence
  Cleavage peptide
KLMHGI

SEQ ID NO: 37 - protein, 6 aa, Artificial Sequence
  Cleavage peptide
LTPLMI

SEQ ID NO: 38 - protein, 6 aa, Artificial Sequence
  Cleavage peptide
SPLMKH

SEQ ID NO: 39 - protein, 6 aa, Artificial Sequence
  Cleavage peptide
YYIAPL

SEQ ID NO: 40 - protein, 6 aa, Artificial Sequence
  Cleavage peptide
KNMMTF

SEQ ID NO: 41 - protein, 6 aa, Artificial Sequence
  Cleavage peptide
FYTKHL

SEQ ID NO: 42 - protein, 6 aa, Artificial Sequence
  Cleavage peptide
MQSAID

SEQ ID NO: 43 - protein, 6 aa, Artificial Sequence
  Cleavage peptide
QFEGVL

SEQ ID NO: 44 - protein, 6 aa, Artificial Sequence
  Cleavage peptide
MQHVIA

SEQ ID NO: 45 - protein, 6 aa, Artificial Sequence
  Cleavage peptide
HNRIEQ

SEQ ID NO: 46 - protein, 6 aa, Artificial Sequence
  Cleavage peptide
RIWKQM

SEQ ID NO: 47 - protein, 6 aa, Artificial Sequence
  Cleavage peptide
KLRQRH

SEQ ID NO: 48 - protein, 6 aa, Artificial Sequence
  Cleavage peptide
WEGLMK

SEQ ID NO: 49 - protein, 6 aa, Artificial Sequence
  Cleavage peptide
FELMKA

SEQ ID NO: 50 - protein, 6 aa, Artificial Sequence
  Cleavage peptide
LMKLHN

SEQ ID NO: 51 - protein, 6 aa, Artificial Sequence
  Cleavage peptide
KHGLMR

SEQ ID NO: 52 - protein, 12 aa, Artificial Sequence
  Cleavage peptide
TLEEWFQVFEFL

SEQ ID NO: 53 - protein, 456 aa, Artificial Sequence
  PBS1-R1 variant
MGCFSCFDSSDDEKLNPVDESNHGQKKQSQPTVSNNISGLPSGGEKLSSKTNGGSKRELLLPRDGLGQIAAHTFAFRELAAATMNFHPDTFLGEGGFGRVYKGRLDSTGQVVAVKQLDRNGLQGNREFLVEVLMLSLLHHPNLVNLIGYCADGDQRLLVYEFMPLGSLEDHLHDLPPDKEALDWNMRMKIAAGAAKGLEFLHDKANPPVIYRDFKSSNILLDEGFHPKLSDFGLAKLGPTRWWFALSTRVMGTYGYCAPEYAMTGQLTVKSDVYSFGVVFLELITGRKAIDSEMPHGEQNLVAWARPLFNDRRKFIKLADPRLKGRFPTRALYQALAVASMCIQEQAATRPLIADVVTALSYLANQAYDPSKDDSRRNRDERGARLITRNDDGGGSGSKFDLEGSEKEDSPRETARILNRDINRERAVAEAKMWGESLREKRRQSEQGTSESNSTG

SEQ ID NO: 54 - protein, 456 aa, Artificial Sequence
  PBS1-R2 variant
MGCFSCFDSSDDEKLNPVDESNHGQKKQSQPTVSNNISGLPSGGEKLSSKTNGGSKRELLLPRDGLGQIAAHTFAFRELAAATMNFHPDTFLGEGGFGRVYKGRLDSTGQVVAVKQLDRNGLQGNREFLVEVLMLSLLHHPNLVNLIGYCADGDQRLLVYEFMPLGSLEDHLHDLPPDKEALDWNMRMKIAAGAAKGLEFLHDKANPPVIYRDFKSSNILLDEGFHPKLSDFGLAKLGPTHWVNFLSTRVMGTYGYCAPEYAMTGQLTVKSDVYSFGVVFLELITGRKAIDSEMPHGEQNLVAWARPLFNDRRKFIKLADPRLKGRFPTRALYQALAVASMCIQEQAATRPLIADVVTALSYLANQAYDPSKDDSRRNRDERGARLITRNDDGGGSGSKFDLEGSEKEDSPRETARILNRDINRERAVAEAKMWGESLREKRRQSEQGTSESNSTG

SEQ ID NO: 55 - protein, 456 aa, Artificial Sequence
  PBS1-R3 variant
MGCFSCFDSSDDEKLNPVDESNHGQKKQSQPTVSNNISGLPSGGEKLSSKTNGGSKRELLLPRDGLGQIAAHTFAFRELAAATMNFHPDTFLGEGGFGRVYKGRLDSTGQVVAVKQLDRNGLQGNREFLVEVLMLSLLHHPNLVNLIGYCADGDQRLLVYEFMPLGSLEDHLHDLPPDKEALDWNMRMKIAAGAAKGLEFLHDKANPPVIYRDFKSSNILLDEGFHPKLSDFGLAKLGPTWAELVLSTRVMGTYGYCAPEYAMTGQLTVKSDVYSFGVVFLELITGRKAIDSEMPHGEQNLVAWARPLFNDRRKFIKLADPRLKGRFPTRALYQALAVASMCIQEQAATRPLIADVVTALSYLANQAYDPSKDDSRRNRDERGARLITRNDDGGGSGSKFDLEGSEKEDSPRETARILNRDINRERAVAEAKMWGESLREKRRQSEQGTSESNSTG

SEQ ID NO: 56 - protein, 456 aa, Artificial Sequence
  PBS1-R4 variant
MGCFSCFDSSDDEKLNPVDESNHGQKKQSQPTVSNNISGLPSGGEKLSSKTNGGSKRELLLPRDGLGQIAAHTFAFRELAAATMNFHPDTFLGEGGFGRVYKGRLDSTGQVVAVKQLDRNGLQGNREFLVEVLMLSLLHHPNLVNLIGYCADGDQRLLVYEFMPLGSLEDHLHDLPPDKEALDWNMRMKIAAGAAKGLEFLHDKANPPVIYRDFKSSNILLDEGFHPKLSDFGLAKLGPTPIISLASTRVMGTYGYCAPEYAMTGQLTVKSDVYSFGVVFLELITGRKAIDSEMPHGEQNLVAWARPLFNDRRKFIKLADPRLKGRFPTRALYQALAVASMCIQEQAATRPLIADVVTALSYLANQAYDPSKDDSRRNRDERGARLITRNDDGGGSGSKFDLEGSEKEDSPRETARILNRDINRERAVAEAKMWGESLREKRRQSEQGTSESNSTG

SEQ ID NO: 57 - protein, 456 aa, Artificial Sequence
  PBS1-R5 variant
MGCFSCFDSSDDEKLNPVDESNHGQKKQSQPTVSNNISGLPSGGEKLSSKTNGGSKRELLLPRDGLGQIAAHTFAFRELAAATMNFHPDTFLGEGGFGRVYKGRLDSTGQVVAVKQLDRNGLQGNREFLVEVLMLSLLHHPNLVNLIGYCADGDQRLLVYEFMPLGSLEDHLHDLPPDKEALDWNMRMKIAAGAAKGLEFLHDKANPPVIYRDFKSSNILLDEGFHPKLSDFGLAKLGPTWFYVLQSTRVMGTYGYCAPEYAMTGQLTVKSDVYSFGVVFLELITGRKAIDSEMPHGEQNLVAWARPLFNDRRKFIKLADPRLKGRFPTRALYQALAVASMCIQEQAATRPLIADVVTALSYLANQAYDPSKDDSRRNRDERGARLITRNDDGGGSGSKFDLEGSEKEDSPRETARILNRDINRERAVAEAKMWGESLREKRRQSEQGTSESNSTG

SEQ ID NO: 58 - protein, 456 aa, Artificial Sequence
  PBS1-R6 variant
MGCFSCFDSSDDEKLNPVDESNHGQKKQSQPTVSNNISGLPSGGEKLSSKTNGGSKRELLLPRDGLGQIAAHTFAFRELAAATMNFHPDTFLGEGGFGRVYKGRLDSTGQVVAVKQLDRNGLQGNREFLVEVLMLSLLHHPNLVNLIGYCADGDQRLLVYEFMPLGSLEDHLHDLPPDKEALDWNMRMKIAAGAAKGLEFLHDKANPPVIYRDFKSSNILLDEGFHPKLSDFGLAKLGPTLTEMFMSTRVMGTYGYCAPEYAMTGQLTVKSDVYSFGVVFLELITGRKAIDSEMPHGEQNLVAWARPLFNDRRKFIKLADPRLKGRFPTRALYQALAVASMCIQEQAATRPLIADVVTALSYLANQAYDPSKDDSRRNRDERGARLITRNDDGGGSGSKFDLEGSEKEDSPRETARILNRDINRERAVAEAKMWGESLREKRRQSEQGTSESNSTG

SEQ ID NO: 59 - protein, 456 aa, Artificial Sequence
  PBS1-R7 variant
MGCFSCFDSSDDEKLNPVDESNHGQKKQSQPTVSNNISGLPSGGEKLSSKTNGGSKRELLLPRDGLGQIAAHTFAFRELAAATMNFHPDTFLGEGGFGRVYKGRLDSTGQVVAVKQLDRNGLQGNREFLVEVLMLSLLHHPNLVNLIGYCADGDQRLLVYEFMPLGSLEDHLHDLPPDKEALDWNMRMKIAAGAAKGLEFLHDKANPPVIYRDFKSSNILLDEGFHPKLSDFGLAKLGPTGQYFVWSTRVMGTYGYCAPEYAMTGQLTVKSDVYSFGVVFLELITGRKAIDSEMPHGEQNLVAWARPLFNDRRKFIKLADPRLKGRFPTRALYQALAVASMCIQEQAATRPLIADVVTALSYLANQAYDPSKDDSRRNRDERGARLITRNDDGGGSGSKFDLEGSEKEDSPRETARILNRDINRERAVAEAKMWGESLREKRRQSEQGTSESNSTG

SEQ ID NO: 60 - protein, 456 aa, Artificial Sequence
  PBS1-R8 variant
MGCFSCFDSSDDEKLNPVDESNHGQKKQSQPTVSNNISGLPSGGEKLSSKTNGGSKRELLLPRDGLGQIAAHTFAFRELAAATMNFHPDTFLGEGGFGRVYKGRLDSTGQVVAVKQLDRNGLQGNREFLVEVLMLSLLHHPNLVNLIGYCADGDQRLLVYEFMPLGSLEDHLHDLPPDKEALDWNMRMKIAAGAAKGLEFLHDKANPPVIYRDFKSSNILLDEGFHPKLSDFGLAKLGPTYWTTLFSTRVMGTYGYCAPEYAMTGQLTVKSDVYSFGVVFLELITGRKAIDSEMPHGEQNLVAWARPLFNDRRKFIKLADPRLKGRFPTRALYQALAVASMCIQEQAATRPLIADVVTALSYLANQAYDPSKDDSRRNRDERGARLITRNDDGGGSGSKFDLEGSEKEDSPRETARILNRDINRERAVAEAKMWGESLREKRRQSEQGTSESNSTG

SEQ ID NO: 61 - protein, 456 aa, Artificial Sequence
  PBS1-R9 variant
MGCFSCFDSSDDEKLNPVDESNHGQKKQSQPTVSNNISGLPSGGEKLSSKTNGGSKRELLLPRDGLGQIAAHTFAFRELAAATMNFHPDTFLGEGGFGRVYKGRLDSTGQVVAVKQLDRNGLQGNREFLVEVLMLSLLHHPNLVNLIGYCADGDQRLLVYEFMPLGSLEDHLHDLPPDKEALDWNMRMKIAAGAAKGLEFLHDKANPPVIYRDFKSSNILLDEGFHPKLSDFGLAKLGPTIQMLWASTRVMGTYGYCAPEYAMTGQLTVKSDVYSFGVVFLELITGRKAIDSEMPHGEQNLVAWARPLFNDRRKFIKLADPRLKGRFPTRALYQALAVASMCIQEQAATRPLIADVVTALSYLANQAYDPSKDDSRRNRDERGARLITRNDDGGGSGSKFDLEGSEKEDSPRETARILNRDINRERAVAEAKMWGESLREKRRQSEQGTSESNSTG

SEQ ID NO: 62 - protein, 456 aa, Artificial Sequence
  PBS1-R10 variant
MGCFSCFDSSDDEKLNPVDESNHGQKKQSQPTVSNNISGLPSGGEKLSSKTNGGSKRELLLPRDGLGQIAAHTFAFRELAAATMNFHPDTFLGEGGFGRVYKGRLDSTGQVVAVKQLDRNGLQGNREFLVEVLMLSLLHHPNLVNLIGYCADGDQRLLVYEFMPLGSLEDHLHDLPPDKEALDWNMRMKIAAGAAKGLEFLHDKANPPVIYRDFKSSNILLDEGFHPKLSDFGLAKLGPTYARFYLSTRVMGTYGYCAPEYAMTGQLTVKSDVYSFGVVFLELITGRKAIDSEMPHGEQNLVAWARPLFNDRRKFIKLADPRLKGRFPTRALYQALAVASMCIQEQAATRPLIADVVTALSYLANQAYDPSKDDSRRNRDERGARLITRNDDGGGSGSKFDLEGSEKEDSPRETARILNRDINRERAVAEAKMWGESLREKRRQSEQGTSESNSTG

SEQ ID NO: 63 - protein, 456 aa, Artificial Sequence
  PBS1-R11 variant
MGCFSCFDSSDDEKLNPVDESNHGQKKQSQPTVSNNISGLPSGGEKLSSKTNGGSKRELLLPRDGLGQIAAHTFAFRELAAATMNFHPDTFLGEGGFGRVYKGRLDSTGQVVAVKQLDRNGLQGNREFLVEVLMLSLLHHPNLVNLIGYCADGDQRLLVYEFMPLGSLEDHLHDLPPDKEALDWNMRMKIAAGAAKGLEFLHDKANPPVIYRDFKSSNILLDEGFHPKLSDFGLAKLGPTLAKLWYSTRVMGTYGYCAPEYAMTGQLTVKSDVYSFGVVFLELITGRKAIDSEMPHGEQNLVAWARPLFNDRRKFIKLADPRLKGRFPTRALYQALAVASMCIQEQAATRPLIADVVTALSYLANQAYDPSKDDSRRNRDERGARLITRNDDGGGSGSKFDLEGSEKEDSPRETARILNRDINRERAVAEAKMWGESLREKRRQSEQGTSESNSTG

SEQ ID NO: 64 - protein, 456 aa, Artificial Sequence
  PBS1-R12 variant
MGCFSCFDSSDDEKLNPVDESNHGQKKQSQPTVSNNISGLPSGGEKLSSKTNGGSKRELLLPRDGLGQIAAHTFAFRELAAATMNFHPDTFLGEGGFGRVYKGRLDSTGQVVAVKQLDRNGLQGNREFLVEVLMLSLLHHPNLVNLIGYCADGDQRLLVYEFMPLGSLEDHLHDLPPDKEALDWNMRMKIAAGAAKGLEFLHDKANPPVIYRDFKSSNILLDEGFHPKLSDFGLAKLGPTYFWLVRSTRVMGTYGYCAPEYAMTGQLTVKSDVYSFGVVFLELITGRKAIDSEMPHGEQNLVAWARPLFNDRRKFIKLADPRLKGRFPTRALYQALAVASMCIQEQAATRPLIADVVTALSYLANQAYDPSKDDSRRNRDERGARLITRNDDGGGSGSKFDLEGSEKEDSPRETARILNRDINRERAVAEAKMWGESLREKRRQSEQGTSESNSTG

SEQ ID NO: 65 - protein, 456 aa, Artificial Sequence
  PBS1-R13 variant
MGCFSCFDSSDDEKLNPVDESNHGQKKQSQPTVSNNISGLPSGGEKLSSKTNGGSKRELLLPRDGLGQIAAHTFAFRELAAATMNFHPDTFLGEGGFGRVYKGRLDSTGQVVAVKQLDRNGLQGNREFLVEVLMLSLLHHPNLVNLIGYCADGDQRLLVYEFMPLGSLEDHLHDLPPDKEALDWNMRMKIAAGAAKGLEFLHDKANPPVIYRDFKSSNILLDEGFHPKLSDFGLAKLGPTGFWLSFSTRVMGTYGYCAPEYAMTGQLTVKSDVYSFGVVFLELITGRKAIDSEMPHGEQNLVAWARPLFNDRRKFIKLADPRLKGRFPTRALYQALAVASMCIQEQAATRPLIADVVTALSYLANQAYDPSKDDSRRNRDERGARLITRNDDGGGSGSKFDLEGSEKEDSPRETARILNRDINRERAVAEAKMWGESLREKRRQSEQGTSESNSTG

SEQ ID NO: 66 - protein, 456 aa, Artificial Sequence
  PBS1-R14 variant
MGCFSCFDSSDDEKLNPVDESNHGQKKQSQPTVSNNISGLPSGGEKLSSKTNGGSKRELLLPRDGLGQIAAHTFAFRELAAATMNFHPDTFLGEGGFGRVYKGRLDSTGQVVAVKQLDRNGLQGNREFLVEVLMLSLLHHPNLVNLIGYCADGDQRLLVYEFMPLGSLEDHLHDLPPDKEALDWNMRMKIAAGAAKGLEFLHDKANPPVIYRDFKSSNILLDEGFHPKLSDFGLAKLGPTTLEEWFSTRVMGTYGYCAPEYAMTGQLTVKSDVYSFGVVFLELITGRKAIDSEMPHGEQNLVAWARPLFNDRRKFIKLADPRLKGRFPTRALYQALAVASMCIQEQAATRPLIADVVTALSYLANQAYDPSKDDSRRNRDERGARLITRNDDGGGSGSKFDLEGSEKEDSPRETARILNRDINRERAVAEAKMWGESLREKRRQSEQGTSESNSTG

SEQ ID NO: 67 - protein, 456 aa, Artificial Sequence
  PBS1-R15 variant
MGCFSCFDSSDDEKLNPVDESNHGQKKQSQPTVSNNISGLPSGGEKLSSKTNGGSKRELLLPRDGLGQIAAHTFAFRELAAATMNFHPDTFLGEGGFGRVYKGRLDSTGQVVAVKQLDRNGLQGNREFLVEVLMLSLLHHPNLVNLIGYCADGDQRLLVYEFMPLGSLEDHLHDLPPDKEALDWNMRMKIAAGAAKGLEFLHDKANPPVIYRDFKSSNILLDEGFHPKLSDFGLAKLGPTQQLFVVSTRVMGTYGYCAPEYAMTGQLTVKSDVYSFGVVFLELITGRKAIDSEMPHGEQNLVAWARPLFNDRRKFIKLADPRLKGRFPTRALYQALAVASMCIQEQAATRPLIADVVTALSYLANQAYDPSKDDSRRNRDERGARLITRNDDGGGSGSKFDLEGSEKEDSPRETARILNRDINRERAVAEAKMWGESLREKRRQSEQGTSESNSTG

SEQ ID NO: 68 - protein, 456 aa, Artificial Sequence
  PBS1-R16 variant
MGCFSCFDSSDDEKLNPVDESNHGQKKQSQPTVSNNISGLPSGGEKLSSKTNGGSKRELLLPRDGLGQIAAHTFAFRELAAATMNFHPDTFLGEGGFGRVYKGRLDSTGQVVAVKQLDRNGLQGNREFLVEVLMLSLLHHPNLVNLIGYCADGDQRLLVYEFMPLGSLEDHLHDLPPDKEALDWNMRMKIAAGAAKGLEFLHDKANPPVIYRDFKSSNILLDEGFHPKLSDFGLAKLGPTMRFYFTSTRVMGTYGYCAPEYAMTGQLTVKSDVYSFGVVFLELITGRKAIDSEMPHGEQNLVAWARPLFNDRRKFIKLADPRLKGRFPTRALYQALAVASMCIQEQAATRPLIADVVTALSYLANQAYDPSKDDSRRNRDERGARLITRNDDGGGSGSKFDLEGSEKEDSPRETARILNRDINRERAVAEAKMWGESLREKRRQSEQGTSESNSTG

SEQ ID NO: 69 - protein, 456 aa, Artificial Sequence
  PBS1-R17 variant
MGCFSCFDSSDDEKLNPVDESNHGQKKQSQPTVSNNISGLPSGGEKLSSKTNGGSKRELLLPRDGLGQIAAHTFAFRELAAATMNFHPDTFLGEGGFGRVYKGRLDSTGQVVAVKQLDRNGLQGNREFLVEVLMLSLLHHPNLVNLIGYCADGDQRLLVYEFMPLGSLEDHLHDLPPDKEALDWNMRMKIAAGAAKGLEFLHDKANPPVIYRDFKSSNILLDEGFHPKLSDFGLAKLGPTQVFEFLSTRVMGTYGYCAPEYAMTGQLTVKSDVYSFGVVFLELITGRKAIDSEMPHGEQNLVAWARPLFNDRRKFIKLADPRLKGRFPTRALYQALAVASMCIQEQAATRPLIADVVTALSYLANQAYDPSKDDSRRNRDERGARLITRNDDGGGSGSKFDLEGSEKEDSPRETARILNRDINRERAVAEAKMWGESLREKRRQSEQGTSESNSTG

SEQ ID NO: 70 - protein, 456 aa, Artificial Sequence
  PBS1-R18 variant
MGCFSCFDSSDDEKLNPVDESNHGQKKQSQPTVSNNISGLPSGGEKLSSKTNGGSKRELLLPRDGLGQIAAHTFAFRELAAATMNFHPDTFLGEGGFGRVYKGRLDSTGQVVAVKQLDRNGLQGNREFLVEVLMLSLLHHPNLVNLIGYCADGDQRLLVYEFMPLGSLEDHLHDLPPDKEALDWNMRMKIAAGAAKGLEFLHDKANPPVIYRDFKSSNILLDEGFHPKLSDFGLAKLGPTVYYFYRSTRVMGTYGYCAPEYAMTGQLTVKSDVYSFGVVFLELITGRKAIDSEMPHGEQNLVAWARPLFNDRRKFIKLADPRLKGRFPTRALYQALAVASMCIQEQAATRPLIADVVTALSYLANQAYDPSKDDSRRNRDERGARLITRNDDGGGSGSKFDLEGSEKEDSPRETARILNRDINRERAVAEAKMWGESLREKRRQSEQGTSESNSTG

SEQ ID NO: 71 - protein, 456 aa, Artificial Sequence
  PBS1-R19 variant
MGCFSCFDSSDDEKLNPVDESNHGQKKQSQPTVSNNISGLPSGGEKLSSKTNGGSKRELLLPRDGLGQIAAHTFAFRELAAATMNFHPDTFLGEGGFGRVYKGRLDSTGQVVAVKQLDRNGLQGNREFLVEVLMLSLLHHPNLVNLIGYCADGDQRLLVYEFMPLGSLEDHLHDLPPDKEALDWNMRMKIAAGAAKGLEFLHDKANPPVIYRDFKSSNILLDEGFHPKLSDFGLAKLGPTQMIFLRSTRVMGTYGYCAPEYAMTGQLTVKSDVYSFGVVFLELITGRKAIDSEMPHGEQNLVAWARPLFNDRRKFIKLADPRLKGRFPTRALYQALAVASMCIQEQAATRPLIADVVTALSYLANQAYDPSKDDSRRNRDERGARLITRNDDGGGSGSKFDLEGSEKEDSPRETARILNRDINRERAVAEAKMWGESLREKRRQSEQGTSESNSTG

SEQ ID NO: 72 - protein, 456 aa, Artificial Sequence
  PBS1-R20 variant
MGCFSCFDSSDDEKLNPVDESNHGQKKQSQPTVSNNISGLPSGGEKLSSKTNGGSKRELLLPRDGLGQIAAHTFAFRELAAATMNFHPDTFLGEGGFGRVYKGRLDSTGQVVAVKQLDRNGLQGNREFLVEVLMLSLLHHPNLVNLIGYCADGDQRLLVYEFMPLGSLEDHLHDLPPDKEALDWNMRMKIAAGAAKGLEFLHDKANPPVIYRDFKSSNILLDEGFHPKLSDFGLAKLGPTIFLWSASTRVMGTYGYCAPEYAMTGQLTVKSDVYSFGVVFLELITGRKAIDSEMPHGEQNLVAWARPLFNDRRKFIKLADPRLKGRFPTRALYQALAVASMCIQEQAATRPLIADVVTALSYLANQAYDPSKDDSRRNRDERGARLITRNDDGGGSGSKFDLEGSEKEDSPRETARILNRDINRERAVAEAKMWGESLREKRRQSEQGTSESNSTG

SEQ ID NO: 73 - protein, 456 aa, Artificial Sequence
  PBS1-R21 variant
MGCFSCFDSSDDEKLNPVDESNHGQKKQSQPTVSNNISGLPSGGEKLSSKTNGGSKRELLLPRDGLGQIAAHTFAFRELAAATMNFHPDTFLGEGGFGRVYKGRLDSTGQVVAVKQLDRNGLQGNREFLVEVLMLSLLHHPNLVNLIGYCADGDQRLLVYEFMPLGSLEDHLHDLPPDKEALDWNMRMKIAAGAAKGLEFLHDKANPPVIYRDFKSSNILLDEGFHPKLSDFGLAKLGPTNLEFLYSTRVMGTYGYCAPEYAMTGQLTVKSDVYSFGVVFLELITGRKAIDSEMPHGEQNLVAWARPLFNDRRKFIKLADPRLKGRFPTRALYQALAVASMCIQEQAATRPLIADVVTALSYLANQAYDPSKDDSRRNRDERGARLITRNDDGGGSGSKFDLEGSEKEDSPRETARILNRDINRERAVAEAKMWGESLREKRRQSEQGTSESNSTG

SEQ ID NO: 74 - protein, 456 aa, Artificial Sequence
  PBS1-R22 variant
MGCFSCFDSSDDEKLNPVDESNHGQKKQSQPTVSNNISGLPSGGEKLSSKTNGGSKRELLLPRDGLGQIAAHTFAFRELAAATMNFHPDTFLGEGGFGRVYKGRLDSTGQVVAVKQLDRNGLQGNREFLVEVLMLSLLHHPNLVNLIGYCADGDQRLLVYEFMPLGSLEDHLHDLPPDKEALDWNMRMKIAAGAAKGLEFLHDKANPPVIYRDFKSSNILLDEGFHPKLSDFGLAKLGPTTFTFFQSTRVMGTYGYCAPEYAMTGQLTVKSDVYSFGVVFLELITGRKAIDSEMPHGEQNLVAWARPLFNDRRKFIKLADPRLKGRFPTRALYQALAVASMCIQEQAATRPLIADVVTALSYLANQAYDPSKDDSRRNRDERGARLITRNDDGGGSGSKFDLEGSEKEDSPRETARILNRDINRERAVAEAKMWGESLREKRRQSEQGTSESNSTG

SEQ ID NO: 75 - protein, 456 aa, Artificial Sequence
  PBS1-S1 variant
MGCFSCFDSSDDEKLNPVDESNHGQKKQSQPTVSNNISGLPSGGEKLSSKTNGGSKRELLLPRDGLGQIAAHTFAFRELAAATMNFHPDTFLGEGGFGRVYKGRLDSTGQVVAVKQLDRNGLQGNREFLVEVLMLSLLHHPNLVNLIGYCADGDQRLLVYEFMPLGSLEDHLHDLPPDKEALDWNMRMKIAAGAAKGLEFLHDKANPPVIYRDFKSSNILLDEGFHPKLSDFGLAKLGPTTAMRIHSTRVMGTYGYCAPEYAMTGQLTVKSDVYSFGVVFLELITGRKAIDSEMPHGEQNLVAWARPLFNDRRKFIKLADPRLKGRFPTRALYQALAVASMCIQEQAATRPLIADVVTALSYLANQAYDPSKDDSRRNRDERGARLITRNDDGGGSGSKFDLEGSEKEDSPRETARILNRDINRERAVAEAKMWGESLREKRRQSEQGTSESNSTG

SEQ ID NO: 76 - protein, 456 aa, Artificial Sequence
  PBS1-S2 variant
MGCFSCFDSSDDEKLNPVDESNHGQKKQSQPTVSNNISGLPSGGEKLSSKTNGGSKRELLLPRDGLGQIAAHTFAFRELAAATMNFHPDTFLGEGGFGRVYKGRLDSTGQVVAVKQLDRNGLQGNREFLVEVLMLSLLHHPNLVNLIGYCADGDQRLLVYEFMPLGSLEDHLHDLPPDKEALDWNMRMKIAAGAAKGLEFLHDKANPPVIYRDFKSSNILLDEGFHPKLSDFGLAKLGPTLIMQKNSTRVMGTYGYCAPEYAMTGQLTVKSDVYSFGVVFLELITGRKAIDSEMPHGEQNLVAWARPLFNDRRKFIKLADPRLKGRFPTRALYQALAVASMCIQEQAATRPLIADVVTALSYLANQAYDPSKDDSRRNRDERGARLITRNDDGGGSGSKFDLEGSEKEDSPRETARILNRDINRERAVAEAKMWGESLREKRRQSEQGTSESNSTG

SEQ ID NO: 77 - protein, 456 aa, Artificial Sequence
  PBS1-S3 variant
MGCFSCFDSSDDEKLNPVDESNHGQKKQSQPTVSNNISGLPSGGEKLSSKTNGGSKRELLLPRDGLGQIAAHTFAFRELAAATMNFHPDTFLGEGGFGRVYKGRLDSTGQVVAVKQLDRNGLQGNREFLVEVLMLSLLHHPNLVNLIGYCADGDQRLLVYEFMPLGSLEDHLHDLPPDKEALDWNMRMKIAAGAAKGLEFLHDKANPPVIYRDFKSSNILLDEGFHPKLSDFGLAKLGPTVMLMHASTRVMGTYGYCAPEYAMTGQLTVKSDVYSFGVVFLELITGRKAIDSEMPHGEQNLVAWARPLFNDRRKFIKLADPRLKGRFPTRALYQALAVASMCIQEQAATRPLIADVVTALSYLANQAYDPSKDDSRRNRDERGARLITRNDDGGGSGSKFDLEGSEKEDSPRETARILNRDINRERAVAEAKMWGESLREKRRQSEQGTSESNSTG

SEQ ID NO: 78 - protein, 456 aa, Artificial Sequence
  PBS1-S4 variant
MGCFSCFDSSDDEKLNPVDESNHGQKKQSQPTVSNNISGLPSGGEKLSSKTNGGSKRELLLPRDGLGQIAAHTFAFRELAAATMNFHPDTFLGEGGFGRVYKGRLDSTGQVVAVKQLDRNGLQGNREFLVEVLMLSLLHHPNLVNLIGYCADGDQRLLVYEFMPLGSLEDHLHDLPPDKEALDWNMRMKIAAGAAKGLEFLHDKANPPVIYRDFKSSNILLDEGFHPKLSDFGLAKLGPTVKSFLMSTRVMGTYGYCAPEYAMTGQLTVKSDVYSFGVVFLELITGRKAIDSEMPHGEQNLVAWARPLFNDRRKFIKLADPRLKGRFPTRALYQALAVASMCIQEQAATRPLIADVVTALSYLANQAYDPSKDDSRRNRDERGARLITRNDDGGGSGSKFDLEGSEKEDSPRETARILNRDINRERAVAEAKMWGESLREKRRQSEQGTSESNSTG

SEQ ID NO: 79 - protein, 456 aa, Artificial Sequence
  PBS1-S5 variant
MGCFSCFDSSDDEKLNPVDESNHGQKKQSQPTVSNNISGLPSGGEKLSSKTNGGSKRELLLPRDGLGQIAAHTFAFRELAAATMNFHPDTFLGEGGFGRVYKGRLDSTGQVVAVKQLDRNGLQGNREFLVEVLMLSLLHHPNLVNLIGYCADGDQRLLVYEFMPLGSLEDHLHDLPPDKEALDWNMRMKIAAGAAKGLEFLHDKANPPVIYRDFKSSNILLDEGFHPKLSDFGLAKLGPTHLYLYHSTRVMGTYGYCAPEYAMTGQLTVKSDVYSFGVVFLELITGRKAIDSEMPHGEQNLVAWARPLFNDRRKFIKLADPRLKGRFPTRALYQALAVASMCIQEQAATRPLIADVVTALSYLANQAYDPSKDDSRRNRDERGARLITRNDDGGGSGSKFDLEGSEKEDSPRETARILNRDINRERAVAEAKMWGESLREKRRQSEQGTSESNSTG

SEQ ID NO: 80 - protein, 456 aa, Artificial Sequence
  PBS1-S6 variant
MGCFSCFDSSDDEKLNPVDESNHGQKKQSQPTVSNNISGLPSGGEKLSSKTNGGSKRELLLPRDGLGQIAAHTFAFRELAAATMNFHPDTFLGEGGFGRVYKGRLDSTGQVVAVKQLDRNGLQGNREFLVEVLMLSLLHHPNLVNLIGYCADGDQRLLVYEFMPLGSLEDHLHDLPPDKEALDWNMRMKIAAGAAKGLEFLHDKANPPVIYRDFKSSNILLDEGFHPKLSDFGLAKLGPTLMFKMPSTRVMGTYGYCAPEYAMTGQLTVKSDVYSFGVVFLELITGRKAIDSEMPHGEQNLVAWARPLFNDRRKFIKLADPRLKGRFPTRALYQALAVASMCIQEQAATRPLIADVVTALSYLANQAYDPSKDDSRRNRDERGARLITRNDDGGGSGSKFDLEGSEKEDSPRETARILNRDINRERAVAEAKMWGESLREKRRQSEQGTSESNSTG

SEQ ID NO: 81 - protein, 456 aa, Artificial Sequence
  PBS1-S7 variant
MGCFSCFDSSDDEKLNPVDESNHGQKKQSQPTVSNNISGLPSGGEKLSSKTNGGSKRELLLPRDGLGQIAAHTFAFRELAAATMNFHPDTFLGEGGFGRVYKGRLDSTGQVVAVKQLDRNGLQGNREFLVEVLMLSLLHHPNLVNLIGYCADGDQRLLVYEFMPLGSLEDHLHDLPPDKEALDWNMRMKIAAGAAKGLEFLHDKANPPVIYRDFKSSNILLDEGFHPKLSDFGLAKLGPTKLMHGISTRVMGTYGYCAPEYAMTGQLTVKSDVYSFGVVFLELITGRKAIDSEMPHGEQNLVAWARPLFNDRRKFIKLADPRLKGRFPTRALYQALAVASMCIQEQAATRPLIADVVTALSYLANQAYDPSKDDSRRNRDERGARLITRNDDGGGSGSKFDLEGSEKEDSPRETARILNRDINRERAVAEAKMWGESLREKRRQSEQGTSESNSTG

SEQ ID NO: 82 - protein, 456 aa, Artificial Sequence
  PBS1-S8 variant
MGCFSCFDSSDDEKLNPVDESNHGQKKQSQPTVSNNISGLPSGGEKLSSKTNGGSKRELLLPRDGLGQIAAHTFAFRELAAATMNFHPDTFLGEGGFGRVYKGRLDSTGQVVAVKQLDRNGLQGNREFLVEVLMLSLLHHPNLVNLIGYCADGDQRLLVYEFMPLGSLEDHLHDLPPDKEALDWNMRMKIAAGAAKGLEFLHDKANPPVIYRDFKSSNILLDEGFHPKLSDFGLAKLGPTLTPLMISTRVMGTYGYCAPEYAMTGQLTVKSDVYSFGVVFLELITGRKAIDSEMPHGEQNLVAWARPLFNDRRKFIKLADPRLKGRFPTRALYQALAVASMCIQEQAATRPLIADVVTALSYLANQAYDPSKDDSRRNRDERGARLITRNDDGGGSGSKFDLEGSEKEDSPRETARILNRDINRERAVAEAKMWGESLREKRRQSEQGTSESNSTG

SEQ ID NO: 83 - protein, 456 aa, Artificial Sequence
  PBS1-S9 variant
MGCFSCFDSSDDEKLNPVDESNHGQKKQSQPTVSNNISGLPSGGEKLSSKTNGGSKRELLLPRDGLGQIAAHTFAFRELAAATMNFHPDTFLGEGGFGRVYKGRLDSTGQVVAVKQLDRNGLQGNREFLVEVLMLSLLHHPNLVNLIGYCADGDQRLLVYEFMPLGSLEDHLHDLPPDKEALDWNMRMKIAAGAAKGLEFLHDKANPPVIYRDFKSSNILLDEGFHPKLSDFGLAKLGPTSPLMKHSTRVMGTYGYCAPEYAMTGQLTVKSDVYSFGVVFLELITGRKAIDSEMPHGEQNLVAWARPLFNDRRKFIKLADPRLKGRFPTRALYQALAVASMCIQEQAATRPLIADVVTALSYLANQAYDPSKDDSRRNRDERGARLITRNDDGGGSGSKFDLEGSEKEDSPRETARILNRDINRERAVAEAKMWGESLREKRRQSEQGTSESNSTG

SEQ ID NO: 84 - protein, 456 aa, Artificial Sequence
  PBS1-S10 variant
MGCFSCFDSSDDEKLNPVDESNHGQKKQSQPTVSNNISGLPSGGEKLSSKTNGGSKRELLLPRDGLGQIAAHTFAFRELAAATMNFHPDTFLGEGGFGRVYKGRLDSTGQVVAVKQLDRNGLQGNREFLVEVLMLSLLHHPNLVNLIGYCADGDQRLLVYEFMPLGSLEDHLHDLPPDKEALDWNMRMKIAAGAAKGLEFLHDKANPPVIYRDFKSSNILLDEGFHPKLSDFGLAKLGPTYYIAPLSTRVMGTYGYCAPEYAMTGQLTVKSDVYSFGVVFLELITGRKAIDSEMPHGEQNLVAWARPLFNDRRKFIKLADPRLKGRFPTRALYQALAVASMCIQEQAATRPLIADVVTALSYLANQAYDPSKDDSRRNRDERGARLITRNDDGGGSGSKFDLEGSEKEDSPRETARILNRDINRERAVAEAKMWGESLREKRRQSEQGTSESNSTG

SEQ ID NO: 85 - protein, 456 aa, Artificial Sequence
  PBS1-S11 variant
MGCFSCFDSSDDEKLNPVDESNHGQKKQSQPTVSNNISGLPSGGEKLSSKTNGGSKRELLLPRDGLGQIAAHTFAFRELAAATMNFHPDTFLGEGGFGRVYKGRLDSTGQVVAVKQLDRNGLQGNREFLVEVLMLSLLHHPNLVNLIGYCADGDQRLLVYEFMPLGSLEDHLHDLPPDKEALDWNMRMKIAAGAAKGLEFLHDKANPPVIYRDFKSSNILLDEGFHPKLSDFGLAKLGPTKNMMTFSTRVMGTYGYCAPEYAMTGQLTVKSDVYSFGVVFLELITGRKAIDSEMPHGEQNLVAWARPLFNDRRKFIKLADPRLKGRFPTRALYQALAVASMCIQEQAATRPLIADVVTALSYLANQAYDPSKDDSRRNRDERGARLITRNDDGGGSGSKFDLEGSEKEDSPRETARILNRDINRERAVAEAKMWGESLREKRRQSEQGTSESNSTG

SEQ ID NO: 86 - protein, 456 aa, Artificial Sequence
  PBS1-S12 variant
MGCFSCFDSSDDEKLNPVDESNHGQKKQSQPTVSNNISGLPSGGEKLSSKTNGGSKRELLLPRDGLGQIAAHTFAFRELAAATMNFHPDTFLGEGGFGRVYKGRLDSTGQVVAVKQLDRNGLQGNREFLVEVLMLSLLHHPNLVNLIGYCADGDQRLLVYEFMPLGSLEDHLHDLPPDKEALDWNMRMKIAAGAAKGLEFLHDKANPPVIYRDFKSSNILLDEGFHPKLSDFGLAKLGPTFYTKHLSTRVMGTYGYCAPEYAMTGQLTVKSDVYSFGVVFLELITGRKAIDSEMPHGEQNLVAWARPLFNDRRKFIKLADPRLKGRFPTRALYQALAVASMCIQEQAATRPLIADVVTALSYLANQAYDPSKDDSRRNRDERGARLITRNDDGGGSGSKFDLEGSEKEDSPRETARILNRDINRERAVAEAKMWGESLREKRRQSEQGTSESNSTG

SEQ ID NO: 87 - protein, 456 aa, Artificial Sequence
  PBS1-S13 variant
MGCFSCFDSSDDEKLNPVDESNHGQKKQSQPTVSNNISGLPSGGEKLSSKTNGGSKRELLLPRDGLGQIAAHTFAFRELAAATMNFHPDTFLGEGGFGRVYKGRLDSTGQVVAVKQLDRNGLQGNREFLVEVLMLSLLHHPNLVNLIGYCADGDQRLLVYEFMPLGSLEDHLHDLPPDKEALDWNMRMKIAAGAAKGLEFLHDKANPPVIYRDFKSSNILLDEGFHPKLSDFGLAKLGPTMQSAIDSTRVMGTYGYCAPEYAMTGQLTVKSDVYSFGVVFLELITGRKAIDSEMPHGEQNLVAWARPLFNDRRKFIKLADPRLKGRFPTRALYQALAVASMCIQEQAATRPLIADVVTALSYLANQAYDPSKDDSRRNRDERGARLITRNDDGGGSGSKFDLEGSEKEDSPRETARILNRDINRERAVAEAKMWGESLREKRRQSEQGTSESNSTG

SEQ ID NO: 88 - protein, 456 aa, Artificial Sequence
  PBS1-S14 variant
MGCFSCFDSSDDEKLNPVDESNHGQKKQSQPTVSNNISGLPSGGEKLSSKTNGGSKRELLLPRDGLGQIAAHTFAFRELAAATMNFHPDTFLGEGGFGRVYKGRLDSTGQVVAVKQLDRNGLQGNREFLVEVLMLSLLHHPNLVNLIGYCADGDQRLLVYEFMPLGSLEDHLHDLPPDKEALDWNMRMKIAAGAAKGLEFLHDKANPPVIYRDFKSSNILLDEGFHPKLSDFGLAKLGPTQFEGVLSTRVMGTYGYCAPEYAMTGQLTVKSDVYSFGVVFLELITGRKAIDSEMPHGEQNLVAWARPLFNDRRKFIKLADPRLKGRFPTRALYQALAVASMCIQEQAATRPLIADVVTALSYLANQAYDPSKDDSRRNRDERGARLITRNDDGGGSGSKFDLEGSEKEDSPRETARILNRDINRERAVAEAKMWGESLREKRRQSEQGTSESNSTG

SEQ ID NO: 89 - protein, 456 aa, Artificial Sequence
  PBS1-S15 variant
MGCFSCFDSSDDEKLNPVDESNHGQKKQSQPTVSNNISGLPSGGEKLSSKTNGGSKRELLLPRDGLGQIAAHTFAFRELAAATMNFHPDTFLGEGGFGRVYKGRLDSTGQVVAVKQLDRNGLQGNREFLVEVLMLSLLHHPNLVNLIGYCADGDQRLLVYEFMPLGSLEDHLHDLPPDKEALDWNMRMKIAAGAAKGLEFLHDKANPPVIYRDFKSSNILLDEGFHPKLSDFGLAKLGPTMQHVIASTRVMGTYGYCAPEYAMTGQLTVKSDVYSFGVVFLELITGRKAIDSEMPHGEQNLVAWARPLFNDRRKFIKLADPRLKGRFPTRALYQALAVASMCIQEQAATRPLIADVVTALSYLANQAYDPSKDDSRRNRDERGARLITRNDDGGGSGSKFDLEGSEKEDSPRETARILNRDINRERAVAEAKMWGESLREKRRQSEQGTSESNSTG

SEQ ID NO: 90 - protein, 456 aa, Artificial Sequence
  PBS1-S16 variant
MGCFSCFDSSDDEKLNPVDESNHGQKKQSQPTVSNNISGLPSGGEKLSSKTNGGSKRELLLPRDGLGQIAAHTFAFRELAAATMNFHPDTFLGEGGFGRVYKGRLDSTGQVVAVKQLDRNGLQGNREFLVEVLMLSLLHHPNLVNLIGYCADGDQRLLVYEFMPLGSLEDHLHDLPPDKEALDWNMRMKIAAGAAKGLEFLHDKANPPVIYRDFKSSNILLDEGFHPKLSDFGLAKLGPTHNRIEQSTRVMGTYGYCAPEYAMTGQLTVKSDVYSFGVVFLELITGRKAIDSEMPHGEQNLVAWARPLFNDRRKFIKLADPRLKGRFPTRALYQALAVASMCIQEQAATRPLIADVVTALSYLANQAYDPSKDDSRRNRDERGARLITRNDDGGGSGSKFDLEGSEKEDSPRETARILNRDINRERAVAEAKMWGESLREKRRQSEQGTSESNSTG

SEQ ID NO: 91 - protein, 456 aa, Artificial Sequence
  PBS1-S17 variant
MGCFSCFDSSDDEKLNPVDESNHGQKKQSQPTVSNNISGLPSGGEKLSSKTNGGSKRELLLPRDGLGQIAAHTFAFRELAAATMNFHPDTFLGEGGFGRVYKGRLDSTGQVVAVKQLDRNGLQGNREFLVEVLMLSLLHHPNLVNLIGYCADGDQRLLVYEFMPLGSLEDHLHDLPPDKEALDWNMRMKIAAGAAKGLEFLHDKANPPVIYRDFKSSNILLDEGFHPKLSDFGLAKLGPTRIWKQMSTRVMGTYGYCAPEYAMTGQLTVKSDVYSFGVVFLELITGRKAIDSEMPHGEQNLVAWARPLFNDRRKFIKLADPRLKGRFPTRALYQALAVASMCIQEQAATRPLIADVVTALSYLANQAYDPSKDDSRRNRDERGARLITRNDDGGGSGSKFDLEGSEKEDSPRETARILNRDINRERAVAEAKMWGESLREKRRQSEQGTSESNSTG

SEQ ID NO: 92 - protein, 456 aa, Artificial Sequence
  PBS1-S18 variant
MGCFSCFDSSDDEKLNPVDESNHGQKKQSQPTVSNNISGLPSGGEKLSSKTNGGSKRELLLPRDGLGQIAAHTFAFRELAAATMNFHPDTFLGEGGFGRVYKGRLDSTGQVVAVKQLDRNGLQGNREFLVEVLMLSLLHHPNLVNLIGYCADGDQRLLVYEFMPLGSLEDHLHDLPPDKEALDWNMRMKIAAGAAKGLEFLHDKANPPVIYRDFKSSNILLDEGFHPKLSDFGLAKLGPTKLRQRHSTRVMGTYGYCAPEYAMTGQLTVKSDVYSFGVVFLELITGRKAIDSEMPHGEQNLVAWARPLFNDRRKFIKLADPRLKGRFPTRALYQALAVASMCIQEQAATRPLIADVVTALSYLANQAYDPSKDDSRRNRDERGARLITRNDDGGGSGSKFDLEGSEKEDSPRETARILNRDINRERAVAEAKMWGESLREKRRQSEQGTSESNSTG

SEQ ID NO: 93 - protein, 456 aa, Artificial Sequence
  PBS1-S20 variant
MGCFSCFDSSDDEKLNPVDESNHGQKKQSQPTVSNNISGLPSGGEKLSSKTNGGSKRELLLPRDGLGQIAAHTFAFRELAAATMNFHPDTFLGEGGFGRVYKGRLDSTGQVVAVKQLDRNGLQGNREFLVEVLMLSLLHHPNLVNLIGYCADGDQRLLVYEFMPLGSLEDHLHDLPPDKEALDWNMRMKIAAGAAKGLEFLHDKANPPVIYRDFKSSNILLDEGFHPKLSDFGLAKLGPTWEGLMKSTRVMGTYGYCAPEYAMTGQLTVKSDVYSFGVVFLELITGRKAIDSEMPHGEQNLVAWARPLFNDRRKFIKLADPRLKGRFPTRALYQALAVASMCIQEQAATRPLIADVVTALSYLANQAYDPSKDDSRRNRDERGARLITRNDDGGGSGSKFDLEGSEKEDSPRETARILNRDINRERAVAEAKMWGESLREKRRQSEQGTSESNSTG

SEQ ID NO: 94 - protein, 456 aa, Artificial Sequence
  PBS1-S21 variant
MGCFSCFDSSDDEKLNPVDESNHGQKKQSQPTVSNNISGLPSGGEKLSSKTNGGSKRELLLPRDGLGQIAAHTFAFRELAAATMNFHPDTFLGEGGFGRVYKGRLDSTGQVVAVKQLDRNGLQGNREFLVEVLMLSLLHHPNLVNLIGYCADGDQRLLVYEFMPLGSLEDHLHDLPPDKEALDWNMRMKIAAGAAKGLEFLHDKANPPVIYRDFKSSNILLDEGFHPKLSDFGLAKLGPTFELMKASTRVMGTYGYCAPEYAMTGQLTVKSDVYSFGVVFLELITGRKAIDSEMPHGEQNLVAWARPLFNDRRKFIKLADPRLKGRFPTRALYQALAVASMCIQEQAATRPLIADVVTALSYLANQAYDPSKDDSRRNRDERGARLITRNDDGGGSGSKFDLEGSEKEDSPRETARILNRDINRERAVAEAKMWGESLREKRRQSEQGTSESNSTG

SEQ ID NO: 95 - protein, 456 aa, Artificial Sequence
  PBS1-S23 variant
MGCFSCFDSSDDEKLNPVDESNHGQKKQSQPTVSNNISGLPSGGEKLSSKTNGGSKRELLLPRDGLGQIAAHTFAFRELAAATMNFHPDTFLGEGGFGRVYKGRLDSTGQVVAVKQLDRNGLQGNREFLVEVLMLSLLHHPNLVNLIGYCADGDQRLLVYEFMPLGSLEDHLHDLPPDKEALDWNMRMKIAAGAAKGLEFLHDKANPPVIYRDFKSSNILLDEGFHPKLSDFGLAKLGPTLMKLHNSTRVMGTYGYCAPEYAMTGQLTVKSDVYSFGVVFLELITGRKAIDSEMPHGEQNLVAWARPLFNDRRKFIKLADPRLKGRFPTRALYQALAVASMCIQEQAATRPLIADVVTALSYLANQAYDPSKDDSRRNRDERGARLITRNDDGGGSGSKFDLEGSEKEDSPRETARILNRDINRERAVAEAKMWGESLREKRRQSEQGTSESNSTG

SEQ ID NO: 96 - protein, 456 aa, Artificial Sequence
  PBS1-S24 variant
MGCFSCFDSSDDEKLNPVDESNHGQKKQSQPTVSNNISGLPSGGEKLSSKTNGGSKRELLLPRDGLGQIAAHTFAFRELAAATMNFHPDTFLGEGGFGRVYKGRLDSTGQVVAVKQLDRNGLQGNREFLVEVLMLSLLHHPNLVNLIGYCADGDQRLLVYEFMPLGSLEDHLHDLPPDKEALDWNMRMKIAAGAAKGLEFLHDKANPPVIYRDFKSSNILLDEGFHPKLSDFGLAKLGPTKHGLMRSTRVMGTYGYCAPEYAMTGQLTVKSDVYSFGVVFLELITGRKAIDSEMPHGEQNLVAWARPLFNDRRKFIKLADPRLKGRFPTRALYQALAVASMCIQEQAATRPLIADVVTALSYLANQAYDPSKDDSRRNRDERGARLITRNDDGGGSGSKFDLEGSEKEDSPRETARILNRDINRERAVAEAKMWGESLREKRRQSEQGTSESNSTG

SEQ ID NO: 97 - protein, 462 aa, Artificial Sequence
  PBS1-R26 variant
MGCFSCFDSSDDEKLNPVDESNHGQKKQSQPTVSNNISGLPSGGEKLSSKTNGGSKRELLLPRDGLGQIAAHTFAFRELAAATMNFHPDTFLGEGGFGRVYKGRLDSTGQVVAVKQLDRNGLQGNREFLVEVLMLSLLHHPNLVNLIGYCADGDQRLLVYEFMPLGSLEDHLHDLPPDKEALDWNMRMKIAAGAAKGLEFLHDKANPPVIYRDFKSSNILLDEGFHPKLSDFGLAKLGPTTLEEWFQVFEFLSTRVMGTYGYCAPEYAMTGQLTVKSDVYSFGVVFLELITGRKAIDSEMPHGEQNLVAWARPLFNDRRKFIKLADPRLKGRFPTRALYQALAVASMCIQEQAATRPLIADVVTALSYLANQAYDPSKDDSRRNRDERGARLITRNDDGGGSGSKFDLEGSEKEDSPRETARILNRDINRERAVAEAKMWGESLREKRRQSEQGTSESNSTG

SEQ ID NO: 98 - protein, 12 aa, Artificial Sequence
  Cleavage peptide
HWVNFLHLYLYH

SEQ ID NO: 99 - protein, 12 aa, Artificial Sequence
  Cleavage peptide
HWVNFLLMFKMP

SEQ ID NO: 100 - protein, 12 aa, Artificial Sequence
  Cleavage peptide
HWVNFLWEGLMK

SEQ ID NO: 101 - protein, 12 aa, Artificial Sequence
  Cleavage peptide
HWVNFLFELMKA

SEQ ID NO: 102 - protein, 12 aa, Artificial Sequence
  Cleavage peptide
HWVNFLLMKLHN

SEQ ID NO: 103 - protein, 12 aa, Artificial Sequence
  Cleavage peptide
HWVNFLKHGLMR

SEQ ID NO: 104 - protein, 12 aa, Artificial Sequence
  Cleavage peptide
YARFYLHLYLYH

SEQ ID NO: 105 - protein, 12 aa, Artificial Sequence
  Cleavage peptide
YARFYLLMFKMP

SEQ ID NO: 106 - protein, 12 aa, Artificial Sequence
  Cleavage peptide
YARFYLWEGLMK

SEQ ID NO: 107 - protein, 12 aa, Artificial Sequence
  Cleavage peptide
YARFYLFELMKA

SEQ ID NO: 108 - protein, 12 aa, Artificial Sequence
  Cleavage peptide
YARFYLLMKLHN

SEQ ID NO: 109 - protein, 12 aa, Artificial Sequence
  Cleavage peptide
YARFYLKHGLMR

SEQ ID NO: 110 - protein, 12 aa, Artificial Sequence
  Cleavage peptide
TLEEWFHLYLYH

SEQ ID NO: 111 - protein, 12 aa, Artificial Sequence
  Cleavage peptide
TLEEWFLMFKMP

SEQ ID NO: 112 - protein, 12 aa, Artificial Sequence
  Cleavage peptide
TLEEWFWEGLMK

SEQ ID NO: 113 - protein, 12 aa, Artificial Sequence
  R14-S21
TLEEWFFELMKA

SEQ ID NO: 114 - protein, 12 aa, Artificial Sequence
  Cleavage peptide
TLEEWFLMKLHN

SEQ ID NO: 115 - protein, 12 aa, Artificial Sequence
  Cleavage peptide
TLEEWFKHGLMR

SEQ ID NO: 116 - protein, 12 aa, Artificial Sequence
  Cleavage peptide
QVFEFLHLYLYH

SEQ ID NO: 117 - protein, 12 aa, Artificial Sequence
  Cleavage peptide
QVFEFLLMFKMP

SEQ ID NO: 118 - protein, 12 aa, Artificial Sequence
  Cleavage peptide
QVFEFLWEGLMK

SEQ ID NO: 119 - protein, 12 aa, Artificial Sequence
  Cleavage peptide
QVFEFLFELMKA

SEQ ID NO: 120 - protein, 12 aa, Artificial Sequence
  Cleavage peptide
QVFEFLLMKLHN

SEQ ID NO: 121 - protein, 12 aa, Artificial Sequence
  Cleavage peptide
QVFEFLKHGLMR

SEQ ID NO: 122 - protein, 12 aa, Artificial Sequence
  Cleavage peptide
QMIFLRHLYLYH

SEQ ID NO: 123 - protein, 12 aa, Artificial Sequence
  Cleavage peptide
QMIFLRLMFKMP

SEQ ID NO: 124 - protein, 12 aa, Artificial Sequence
  Cleavage peptide
QMIFLRWEGLMK

SEQ ID NO: 125 - protein, 12 aa, Artificial Sequence
  R19-S21
QMIFLRFELMKA

SEQ ID NO: 126 - protein, 12 aa, Artificial Sequence
  Cleavage peptide
QMIFLRLMKLHN

SEQ ID NO: 127 - protein, 12 aa, Artificial Sequence
  Cleavage peptide
QMIFLRKHGLMR

SEQ ID NO: 128 - protein, 12 aa, Artificial Sequence
  Cleavage peptide
IFLWSAHLYLYH

SEQ ID NO: 129 - protein, 12 aa, Artificial Sequence
  Cleavage peptide
IFLWSALMFKMP

SEQ ID NO: 130 - protein, 12 aa, Artificial Sequence
  Cleavage peptide
IFLWSAWEGLMK

SEQ ID NO: 131 - protein, 12 aa, Artificial Sequence
  Cleavage peptide
IFLWSAFELMKA

SEQ ID NO: 132 - protein, 12 aa, Artificial Sequence
  Cleavage peptide
IFLWSALMKLHN

SEQ ID NO: 133 - protein, 12 aa, Artificial Sequence
  Cleavage peptide
IFLWSAKHGLMR

SEQ ID NO: 134 - protein, 12 aa, Artificial Sequence
  Cleavage peptide
NLEFLYHLYLYH

SEQ ID NO: 135 - protein, 12 aa, Artificial Sequence
  Cleavage peptide
NLEFLYLMFKMP

SEQ ID NO: 136 - protein, 12 aa, Artificial Sequence
  Cleavage peptide
NLEFLYWEGLMK

SEQ ID NO: 137 - protein, 12 aa, Artificial Sequence
  Cleavage peptide
NLEFLYFELMKA

SEQ ID NO: 138 - protein, 12 aa, Artificial Sequence
  Cleavage peptide
NLEFLYLMKLHN

SEQ ID NO: 139 - protein, 12 aa, Artificial Sequence
  Cleavage peptide
NLEFLYKHGLMR

SEQ ID NO: 140 - protein, 12 aa, Artificial Sequence
  Cleavage peptide
TFTFFQHLYLYH

SEQ ID NO: 141 - protein, 12 aa, Artificial Sequence
  Cleavage peptide
TFTFFQLMFKMP

SEQ ID NO: 142 - protein, 12 aa, Artificial Sequence
  Cleavage peptide
TFTFFQWEGLMK

SEQ ID NO: 143 - protein, 12 aa, Artificial Sequence
  Cleavage peptide
TFTFFQFELMKA

SEQ ID NO: 144 - protein, 12 aa, Artificial Sequence
  Cleavage peptide
TFTFFQLMKLHN

SEQ ID NO: 145 - protein, 12 aa, Artificial Sequence
  Cleavage peptide
TFTFFQKHGLMR

SEQ ID NO: 146 - protein, 6 aa, Artificial Sequence
  Soy rust motif
  MISC_FEATURE (1)..(1): X is R, H, W, P, L, G, Y, I, T, Q, M, V, or N
  MISC_FEATURE (2)..(2): X is L or Y
  MISC_FEATURE (3)..(3): X is W, V, E, I, Y, T, M, R, K, L, or F
  MISC_FEATURE (4)..(4): X is F or W
  MISC_FEATURE (5)..(5): X is A, F, V, L, W, Y, or S
  MISC_FEATURE (6)..(6): X is L, A, Q, M, W, F, Y, R, V, or T
XXXXXX

SEQ ID NO: 147 - protein, 6 aa, Artificial Sequence
  Nematode motif
  MISC_FEATURE (1)..(1): X is T, L, V, H, K, S, Y, F, M, Q, R or W
  MISC_FEATURE (2)..(2): X is A, I, M, K, L, T, P, Y, N, Q, F, E, or H
  MISC_FEATURE (5)..(5): X is I, K, H, L, Y, M, G, P, T, V, E, Q, or R
  MISC_FEATURE (6)..(6): X is H, N, A, M, P, I, F, L, D, Q, K, or R
XXLMXX

SEQ ID NO: 148 - protein, 211 aa, Arabidopsis thaliana
MARSNVPKFGNWEAEENVPYTAYFDKARKTRAPGSKIMNPNDPEYNSDSQSQAPPHPPSSRTKPEQVDTVRRSREHMRSREESELKQFGDAGGSSNEAANKRQGRASQNNSYDNKSPLHKNSYDGTGKSRPKPTNLRADESPEKVTVVPKFGDWDENNPSSADGYTHIFNKVREERSSGANVSGSSRTPTHQSSRNPNNTSSCCCFGFGGK

SEQ ID NO: 149 - DNA, 633 bp, Arabidopsis thaliana
atggcgcgcagcaacgtgccgaaatttggcaactgggaagcggaagaaaacgtgccgtataccgcgtattttgataaagcgcgcaaaacccgcgcgccgggcagcaaaattatgaacccgaacgatccggaatataacagcgatagccagagccaggcgccgccgcatccgccgagcagccgcaccaaaccggaacaggtggataccgtgcgccgcagccgcgaacatatgcgcagccgcgaagaaagcgaactgaaacagtttggcgatgcgggcggcagcagcaacgaagcggcgaacaaacgccagggccgcgcgagccagaacaacagctatgataacaaaagcccgctgcataaaaacagctatgatggcaccggcaaaagccgcccgaaaccgaccaacctgcgcgcggatgaaagcccggaaaaagtgaccgtggtgccgaaatttggcgattgggatgaaaacaacccgagcagcgcggatggctatacccatatttttaacaaagtgcgcgaagaacgcagcagcggcgcgaacgtgagcggcagcagccgcaccccgacccatcagagcagccgcaacccgaacaacaccagcagctgctgctgctttggctttggcggcaaa

SEQ ID NO: 150 - protein, 466 aa, Glycine max
MGCFSCFDSREDEKLNPNPQQENHQHEHEHEHDLKPPVPSRISRLPPSASASASASAVGADKLRSTTSNGNGESTAVQIAAQTFSFRELAAATKNFRPQSFLGEGGFGRVYKGRLETTGQVVAVKQLDRNGLQGNREFLVEVLMLSLLHHPNLVNLIGYCADGDQRLLVYEFMPFGSLEDHLHDLPPDKEPLDWNTRMKIAAGAAKGLEYLHDKANPPVIYRDFKSSNILLDEGYHPKLSDFGLAKLGPVGDKSHVSTRVMGTYGYCAPEYAMTGQLTVKSDVYSFGVVFLELITGRKAIDSTRPHGEQNLVTWARPLFSDRRKFPKLADPQLQGRYPMRGLYQALAVASMCIQEQAAARPLIGDVVTALSFLANQAYDHRGAGDDKKNRDDKGGRILKNDVGGGSGRRWDLEGSEKDDSPRETARMLNNRDLDRERAVAEAKIWGENWREKRRQSAQGSFDGSNA

SEQ ID NO: 151 - protein, 468 aa, Glycine max
MGCFSCFDSREDEKLNPNPQQENHQHEHEHEHEHDLKPPVPSRISRLPPSASASASASAVGADKLRSTTSNGNGESTAVQIAAQTFSFRELAAATKNFRPQSFLGEGGFGRVYKGRLETTGQVVAVKQLDRNGLQGNREFLVEVLMLSLLHHPNLVNLIGYCADGDQRLLVYEFMPFGSLEDHLHDLPPDKEPLDWNTRMKIAAGAAKGLEYLHDKANPPVIYRDFKSSNILLDEGYHPKLSDFGLAKLGPVGDKSHVSTRVMGTYGYCAPEYAMTGQLTVKSDVYSFGVVFLELITGRKAIDSTRPHGEQNLVTWARPLFSDRRKFPKLADPQLQGRYPMRGLYQALAVASMCIQEQAAARPLIGDVVTALSFLANQAYDHRGAGDDKKNRDDKGGRILKNDVGGGSGRRWDLEGSEKDDSPRETARMLNNRDLDRERAVAEAKIWGENWREKRRQSAQGSFDGSNA

SEQ ID NO: 152 - protein, 468 aa, Glycine max; identical to SEQ ID NO: 151

SEQ ID NO: 153 - DNA, 2046 bp, Arabidopsis thaliana
gtcaacgagaaaagtttcgaaggaaggatttactgagagtttcagcaacgcgtcattgttaattcgcaattggtaatagtgtgaacactcgtggacgacaaaagaagctgcttcttcatctctctctctctccttctctttctctctcaactccaattcgtttttaccagtttcgaatctgaacaaagttgggttttttattggtacccagaatctcaattctccttctttccttctgggtataggaaaagtctcgcctttttttatatttatccaatcgctcctgttcattgattccccagtaggattgtagtttttggttattgggaaaattgagtaaaggaggagatgggttgtttctcgtgttttgattcgagtgatgacgagaagctgaatccagttgatgaatctaatcatggtcagaagaaacaatcacaaccgacagtatccaataacatatctggactcccttcaggtggggagaagcttagctcaaagaccaatggaggatcaaaaagggagctactgcttccaagggatggacttggacaaattgctgctcatacatttgctttccgcgagcttgctgctgcaactatgaactttcatcctgacactttcttaggcgaaggtggatttggacgtgtctacaaaggaaggcttgacagcaccggtcaggttgttgctgttaaacaactagacaggaatggtctacaaggtaacagagaatttctggtagaggttcttatgctcagtcttcttcatcatcccaacttagtcaaccttattggttattgtgctgatggagatcaacgcctcttggtctacgagtttatgccgttaggatcattggaagatcacctccacgatcttccaccggataaggaggccttagattggaacatgagaatgaaaatagctgctggtgcggcgaaaggattggaatttctacatgataaggcaaaccctccggttatttatagagattttaagtcatcaaatattttactggatgagggtttccaccctaagctttctgattttggacttgctaaactcggaccaacgggagacaaatctcatgtctccactagagttatgggaacttatggttattgtgctcccgagtacgcaatgacgggacaattgacagtaaaatcagatgtctacagttttggtgtggtttttctcgagctcattactggtcgcaaagctatagacagcgagatgcctcatggagagcagaacctggtggcttgggctcgcccattgttcaacgacaggcgaaagttcataaaactggctgatccaaggttaaaggggcggtttccaacgcgtgcactctaccaagctttagctgtggcatcaatgtgcatccaagaacaggcggctacacgtcctctcatagcagatgttgtcactgcactctcctatcttgcaaaccaagcttatgatccaagtaaagatgatagtagaagaaaccgggatgaaagaggtgcaaggttaataacaaggaacgacgatggaggtggctcgggaagtaaattcgatttagaaggttcagagaaagaagattcaccgagagagacagctcggatattgaaccgagatatcaatagggagcgtgcggttgcagaggctaagatgtggggagagagtttgagggagaaacgaagacagagcgagcagggtacttcagagagcaacagtaccgggtagaaccggtttgggtcggtcttgacccttttttgttcttatttctctcaagactctcatttattgtcaaacatagaaaacaaacaaaaaaacattgggacgacgagttgctgtacattatatatgtgatgttgcaatatgggcacaaaggttaataaccaaaactctctaattgagattctggagagttgtgaagattgttcatgtaaattttgtagtctttctatttttaaattttgacagatatgtgtttcatgatgagtcacatgtggcgatgtttacgtctcattgaaagattgaatagcaaagatccaatattaagtgtttctg

(19) United States
(12) Patent Application Publication
(10) Pub. No.: US 2024/0060082 A1
(43) Pub. Date: Feb. 22, 2024

(54) Soybean Engineered Resistance

(71) Applicant: Syngenta Crop Protection AG, Basel (CH)

(72) Inventors: Qingli Liu, Research Triangle Park, NC (US); Clarence Michael Reynolds, Research Triangle Park, NC (US); Xiaoping Tan, Research Triangle Park, NC (US); Milan Jucovic, Research Triangle Park, NC (US)

(73) Assignee: Syngenta Crop Protection AG, Basel (CH)

(21) Appl. No.: 18/271,339

(22) PCT Filed: Jan. 14, 2022
(86) PCT No.: PCT/US22/12453

§ 371 Date: Jul. 7, 2023

Related U.S. Application Data
(60) Provisional application No. 63/140,539, filed on Jan. 22, 2021.

(51) Int. Cl. C12N 15/82

(52) U.S. Cl. CPC C12N 15/8282

(57) ABSTRACT

The present invention provides compositions, systems, and methods for conferring resistance to plant pathogens that express pathogen-specific proteases. Compositions of the invention may include a recombinant nucleic acid molecule comprising a promoter operably linked to a nucleotide sequence that encodes at least one substrate protein of a plant pathogen-specific protease expressed by a Phakopsora or Heterodera plant pathogen species. Additionally, the at least one substrate protein has a Phakopsora-specific or Heterodera-specific heterologous cleavage site, wherein cleavage of the cleavage site confers improved resistance to the Phakopsora or Heterodera plant pathogen species.

### RELATED APPLICATION

This application claims priority to U.S. Patent Application No. 63/140,539, filed 22 Jan. 2021, the entire contents of which are incorporated by reference herein.

### FIELD OF THE INVENTION

This invention relates generally to the field of plant genetics and plant molecular biology. More specifically, this invention relates to compositions comprising novel substrate proteins engineered for recognition by plant pathogen-specific proteases, and methods of use of novel substrate proteins in conferring disease resistance to plant pathogens.

### SEQUENCE LISTING

This application is accompanied by a sequence listing entitled 82202_WO_ST25.txt, created Dec. 13, 2021, which is approximately 234 kilobytes in size. This sequence listing is incorporated herein by reference in its entirety. This sequence listing is submitted herewith via EFS-Web and is in compliance with 37 C.F.R. § 1.824(a)(2)-(6) and (b).

### BACKGROUND

Soybean is one of the most important agricultural crops in the world. It is economically vital as it serves as a major source for numerous areas such as food, protein, oil, and other soy products. There are numerous pathogens that threaten soybean production (for example, fungal pathogens, bacterial pathogens, and nematodes). Asian Soybean Rust (ASR), caused by the fungal pathogen Phakopsora pachyrhizi, is one of the most damaging diseases affecting legume crops. This aggressive pathogen originated in eastern Asia and was first detected in the continental United States in 2004. It is a parasite that destroys a plant's leaves. P. pachyrhizi can infect more than 95 species, mostly legumes; alternative hosts serve as a reservoir for inoculum build up. Pathogen infection is quick as the spores can infect directly without need for a wound or opening. If temperature and moisture conditions are optimal, infection can occur within 6 hours. Infected plant leaves develop water-soaked spots that progress to reddish brown or tan lesions. The infected foliage turns bronze/yellow and premature defoliation can occur as a result, ultimately affecting the number of pods and seed weight. The spores are spread aerially and under most optimal conditions, a plant can go from first signs of infection to severe defoliation in 1-2 weeks.

Yield losses as high as 80% have been reported due to ASR (See, Kawashima et al. (2016) Nat. Biotechnol. 34:661-65). More specifically, ASR is devastating in Latin America, estimated to cause approximately $2 billion of damage in Brazil. There are several main control measures utilized for ASR: crop monitoring, chemical fungicides, breeding resistant soybean cultivars, and specific cultivation practices. Incidence of plant diseases can be controlled by agronomic practices that include conventional breeding techniques, crop rotation, and use of synthetic agrochemicals. Conventional breeding methods, however, are time-consuming and require continuous effort to maintain disease resistance as plant pathogens evolve. See, Grover and Gowthaman (2003) Curr. Sci. 84:330-340. Likewise, agrochemicals increase costs to farmers and cause harmful effects on the ecosystem. Because of such concerns, regulators have banned or limited the use of some of the most harmful agrochemicals.

Plant parasitic nematodes are also damaging to soybean, as well as other crops. Agriculturally important nematodes include species from, for example, Heterodera, Globodera, Meloidogyne, and Rotylenchulus. Heterodera glycines, also known as Soybean Cyst Nematode, is responsible for a disease producing Soybean cysts. These pests are soil-borne and many of them attack the roots of various crops. Unfortunately, damage and loss due to nematodes is not easily detectible early in the growing season. As with rusts and other diseases on agricultural crops, methods to reduce nematode infestation, damage, and yield loss are important.

Plants have innate immune responses that can provide some degree of disease resistance against certain plant pathogens. Natural variation for resistance to plant pathogens has been identified by plant breeders and pathologists and can be bred into many plants. One component of plant innate immune response to pathogens includes natural disease resistance genes (or R genes) that provide high levels of resistance (or immunity) to particular plant pathogens and represent an economical and environmentally friendly form of plant protection. Innate disease resistance in plants to plant pathogens typically is governed by the presence of dominant or semidominant disease resistance (R) genes in the plant and dominant avirulence (avr) genes in the pathogen.

Agricultural scientists can now enhance plant pathogen resistance by genetically engineering plants to express anti-pathogen polypeptides. For example, potato and tobacco plants have been developed that exhibit an increased resistance to foliar and soil-borne fungal pathogens. See, Lorito et al. (1998) Proc. Natl. Acad. Sci. USA 95:7860-7865. In addition, transgenic barley has been developed that exhibit an increased resistance to fungal pathogens. See, Horvath et al. (2003) Proc. Natl. Acad. Sci. USA 100:364-369. Moreover, transgenic corn and cotton plants have been developed to produce Cry endotoxins. See, e.g., Aronson (2002) Cell Mol. Life Sci. 59:417-425; and Schnepf et al. (1998) Microbiol. Mol. Biol. Rev. 62:775-806. Other crops, including potatoes, have been genetically engineered to contain similar endotoxins. See, Hussein et al. (2006) J. Chem. Ecol. 32:1-8; Kalushkov and Nedved (2005) J. Appl. Entomol. 129:401-406 and Dangl et al. (2013) Science 341: 746-751. Soy rust resistance traits in commercial soybean lines deployed in the field have gradually been overcome by soy rust. Considering the significant impact of plant pathogens such as Asian soybean rust and Soybean Cyst Nematode, on the yield and quality of plants, additional compositions, systems and methods for protecting plants from plant pathogens are needed.

### SUMMARY

The present invention provides compositions, systems, and methods for conferring resistance to plant pathogens that express pathogen-specific proteases. Plant pathogens use pathogen-specific proteases as virulence factors for infecting host plants. Various embodiments of the invention include modifying at least one member of a protein pair used by plants to detect the pathogen-specific proteases. These protein pairs activate endogenous defense systems in plants upon recognition of the pathogen-specific proteases. Typically, such protein pairs comprise one member that is a disease resistance protein (such as the product of an R gene) and another member that is a substrate protein of the pathogen-specific protease (such as the product of a corresponding Avr gene). As illustrated at FIG. 5, following cleavage of the substrate protein by the pathogen-specific protease at a cleavage site, the substrate protein interacts with the disease resistance protein (such as via physical association), and the coordinated action of the protein pair elicits a localized immune response against the pathogen in the plant (e.g., a hypersensitive programmed cell death response in infected plant cells/tissues). The inventors herein have recognized that the specificity of such pairs for a given pathogen-specific protease can be modified by replacing an endogenous protease recognition sequence or cleavage site in the substrate protein with a heterologous protease cleavage site that corresponds to a pathogen-specific protease of interest (i.e., a protease of a target pathogen against which enhanced resistance is desired).

The compositions of the invention include a recombinant nucleic acid comprising a nucleotide sequence that encodes at least one modified hypersensitive response substrate (HRS) protein of a plant pathogen-specific protease comprising a heterologous cleavage site (in place of an endogenous cleavage site). As a result of the modification, the substrate protein can be recognized and cleaved by a pathogen-specific protease of interest, such as a protease derived from a target pathogen that is different from the endogenous protease of a natural pathogen to which the substrate protein inherently binds, thereby conferring a plant expressing the modified substrate protein with increased resistance to the target pathogen. In one particular embodiment, the protease of interest is one expressed by a Basidiomycete plant pathogen species and, the at least one modified substrate protein is modified to have a Basidiomycete plant pathogen specific heterologous cleavage site in place of the endogenous cleavage site, wherein cleavage of the modified HRS protein at the heterologous cleavage site confers improved resistance to the Basidiomycete plant pathogen species. In another particular embodiment, the protease of interest is one expressed by a Nematoda (e.g., Heterodera) plant pathogen species and, the at least one modified substrate protein is modified to have a Nematoda plant pathogen specific heterologous cleavage site in place of the endogenous cleavage site, wherein cleavage of the modified HRS protein at the heterologous cleavage site confers improved resistance to the Nematoda plant pathogen species. The HRS protein may be derived from Arabidopsis, Glycine, Hordeum, or Triticum. In particular embodiments, the HRS protein is PBS1, RIN4, or a polypeptide having at least 80%, at least 85%, at least 90%, at least 95%, at least 96%, at least 96%, at least 98% or at least 99% sequence identity to PBS1 or RIN4 and retaining substate protein activity. The HRS proteins of a plant interact with disease resistance proteins (R proteins) encoded by R genes of the plant to provide an innate immune response against a pathogen. Various R genes have been discovered in plants, such as RPM1, RPS2 and RPS5, and are known to confer resistance to plant pathogens through the specific recognition of plant pathogen specific proteases, such as the protease AvrRpt2 and AvrPphB in Arabidopsis. The recognition of the protease requires interaction between substrate proteins such as PBS1 or RIN4 and resistance proteins such as the product of the RPS5 or RPM1 genes. Accordingly, in embodiments, the compositions of the present invention include nucleic acids comprising a nucleic acid sequence encoding a modified PBS1 or RIN4 protein wherein the endogenous cleavage site of PBS1 or RIN4 is modified with a heterogenous cleavage site. In particular embodiments, the endogenous cleavage site of PBS1 or RIN4 is modified to comprise a heterogenous cleavage site for a Basidiomycetes species plant pathogen, such as a Phakopsora species pathogen, or a Nematoda species plant pathogen, such as a Heterodera species pathogen.

In other embodiments, the nucleotide sequence that encodes the hypersensitive response substrate (HRS) protein is operably linked to a promoter active in a plant. In further embodiments, the recombinant nucleic acid further comprises an expression cassette comprising a promoter active in a plant operably linked to an R-gene that is activated by the HRS protein following cleavage at the heterologous (e.g., Basidiomycete-specific or Nematoda-specific) protease cleavage site.

In still further embodiments, the invention comprises a vector, a transformed plant cell, and a transformed plant comprising the recombinant nucleic acid molecule encoding the modified HRS protein comprising the heterogenous cleavage site for a pathogen-specific protease. Optionally, the transformed plant cell and transformed plant is a dicot and furthermore a member of the genus Glycine. The invention also covers a transgenic seed or other plant part of the transformed plant.

In some embodiments, the invention provides a method of protecting a plant from infection by a plant pathogen species. In particular embodiments, the method protects the plant from infection against a target plant species against which the plant does not have innate immunity (e.g., against which the plant does not elicit a defense response). In one example, a method comprises the step of introducing into the plant a nucleotide sequence that encodes at least one hypersensitive response substrate protein of a plant pathogen-specific protease expressed by the target plant pathogen species. The at least one substrate protein has a pathogen-specific heterologous cleavage site and cleavage of the heterologous cleavage site by the protease of the target pathogen confers resistance to the target plant pathogen species. The heterologous cleavage site of the substrate protein is engineered to be specific to the target plant pathogen species and is modified from an endogenous cleavage site of the plant that is not specific to the target plant pathogen species. In a particular embodiment of the previously mentioned method, the plant pathogen species is a Phakopsora plant pathogen and is optionally Phakopsora pachyrhizi causing the disease Asian Soybean Rust. In another embodiment, the plant pathogen species is a Nematoda plant pathogen and is optionally a member of Heterodera, Globodera, Meloidogyne, or Rotylenchulus. In a particular embodiment, the plant pathogen species is the soybean cyst nematode Heterodera glycines.

### BRIEF DESCRIPTION OF THE SEQUENCES IN THE SEQUENCE LISTING

SEQ ID NO: 1 is an amino acid sequence of Arabidopsis thaliana PBS1.

SEQ ID NO: 2 is a nucleotide sequence of Arabidopsis thaliana PBS1.

SEQ ID NO: 3 is an amino acid sequence of RPS5 protein from A. thaliana.

SEQ ID NO: 4 is a nucleotide sequence of RPS5 protein from A. thaliana

SEQ ID NO: 5 is a nucleotide sequence of a prGmUbi promoter used to drive vectors with various PBS1 variants.

SEQ ID NO: 6 is a nucleotide sequence of a prMt12344 promoter used to drive vectors containing RPS5.

SEQ ID NO: 7 is an amino acid sequence of the endogenous cleavage site (AvrPphB).

SEQ ID NO: 8 is the amino acid sequence of the R1 cleavage site peptide.

SEQ ID NO: 9 is the amino acid sequence of the R2 cleavage site peptide.

SEQ ID NO: 10 is the amino acid sequence of the R3 cleavage site peptide.

SEQ ID NO: 11 is the amino acid sequence of the R4 cleavage site peptide.

SEQ ID NO: 12 is the amino acid sequence of the R5 cleavage site peptide.

SEQ ID NO: 13 is the amino acid sequence of the R6 cleavage site peptide.

SEQ ID NO: 14 is the amino acid sequence of the R7 cleavage site peptide.

SEQ ID NO: 15 is the amino acid sequence of the R8 cleavage site peptide.

SEQ ID NO: 16 is the amino acid sequence of the R9 cleavage site peptide.

SEQ ID NO: 17 is the amino acid sequence of the R10 cleavage site peptide.

SEQ ID NO: 18 is the amino acid sequence of the R11 cleavage site peptide.

SEQ ID NO: 19 is the amino acid sequence of the R12 cleavage site peptide.

SEQ ID NO: 20 is the amino acid sequence of the R13 cleavage site peptide.

SEQ ID NO: 21 is the amino acid sequence of the R14 cleavage site peptide.

SEQ ID NO: 22 is the amino acid sequence of the R15 cleavage site peptide.

SEQ ID NO: 23 is the amino acid sequence of the R16 cleavage site peptide.

SEQ ID NO: 24 is the amino acid sequence of the R17 cleavage site peptide.

SEQ ID NO: 25 is the amino acid sequence of the R18 cleavage site peptide.

SEQ ID NO: 26 is the amino acid sequence of the R19 cleavage site peptide.

SEQ ID NO: 27 is the amino acid sequence of the R20 cleavage site peptide.

SEQ ID NO: 28 is the amino acid sequence of the R21 cleavage site peptide.

SEQ ID NO: 29 is the amino acid sequence of the R22 cleavage site peptide.

SEQ ID NO: 30 is the amino acid sequence of the S1 cleavage site peptide.

SEQ ID NO: 31 is the amino acid sequence of the S2 cleavage site peptide.

SEQ ID NO: 32 is the amino acid sequence of the S3 cleavage site peptide.

SEQ ID NO: 33 is the amino acid sequence of the S4 cleavage site peptide.

SEQ ID NO: 34 is the amino acid sequence of the S5 cleavage site peptide.

SEQ ID NO: 35 is the amino acid sequence of the S6 cleavage site peptide.

SEQ ID NO: 36 is the amino acid sequence of the S7 cleavage site peptide.

SEQ ID NO: 37 is the amino acid sequence of the S8 cleavage site peptide.

SEQ ID NO: 38 is the amino acid sequence of the S9 cleavage site peptide.

SEQ ID NO: 39 is the amino acid sequence of the S10 cleavage site peptide.

SEQ ID NO: 40 is the amino acid sequence of the S11 cleavage site peptide.

SEQ ID NO: 41 is the amino acid sequence of the S12 cleavage site peptide.

SEQ ID NO: 42 is the amino acid sequence of the S13 cleavage site peptide.

SEQ ID NO: 43 is the amino acid sequence of the S14 cleavage site peptide.

SEQ ID NO: 44 is the amino acid sequence of the S15 cleavage site peptide.

SEQ ID NO: 45 is the amino acid sequence of the S16 cleavage site peptide.

SEQ ID NO: 46 is the amino acid sequence of the S17 cleavage site peptide.

SEQ ID NO: 47 is the amino acid sequence of the S18 cleavage site peptide.

SEQ ID NO: 48 is the amino acid sequence of the S20 cleavage site peptide.

SEQ ID NO: 49 is the amino acid sequence of the S21 cleavage site peptide.

SEQ ID NO: 50 is the amino acid sequence of the S23 cleavage site peptide.

SEQ ID NO: 51 is the amino acid sequence of the S24 cleavage site peptide.

SEQ ID NO: 52 is the amino acid sequence of the R26 (concatenated) cleavage site peptide.

SEQ ID NO: 53 is a modified PBS1 amino acid sequence; it is modified to replace the endogenous cleavage site (SEQ ID NO: 7) with cleavage site R1 (SEQ ID NO: 8).

SEQ ID NO: 54 is a modified PBS1 amino acid sequence; it is modified to replace the endogenous cleavage site (SEQ ID NO: 7) with cleavage site R2 (SEQ ID NO: 9).

SEQ ID NO: 55 is a modified PBS1 amino acid sequence; it is modified to replace the endogenous cleavage site (SEQ ID NO: 7) with cleavage site R3 (SEQ ID NO: 10).

SEQ ID NO: 56 is a modified PBS1 amino acid sequence; it is modified to replace the endogenous cleavage site (SEQ ID NO: 7) with cleavage site R4 (SEQ ID NO: 11).

SEQ ID NO: 57 is a modified PBS1 amino acid sequence; it is modified to replace the endogenous cleavage site (SEQ ID NO: 7) with cleavage site R5 (SEQ ID NO: 12).

SEQ ID NO: 58 is a modified PBS1 amino acid sequence; it is modified to replace the endogenous cleavage site (SEQ ID NO: 7) with cleavage site R6 (SEQ ID NO: 13).

SEQ ID NO: 59 is a modified PBS1 amino acid sequence; it is modified to replace the endogenous cleavage site (SEQ ID NO: 7) with cleavage site R7 (SEQ ID NO: 14).

SEQ ID NO: 60 is a modified PBS1 amino acid sequence; it is modified to replace the endogenous cleavage site (SEQ ID NO: 7) with cleavage site R8 (SEQ ID NO: 15).

SEQ ID NO: 61 is a modified PBS1 amino acid sequence; it is modified to replace the endogenous cleavage site (SEQ ID NO: 7) with cleavage site R9 (SEQ ID NO: 16).

SEQ ID NO: 62 is a modified PBS1 amino acid sequence; it is modified to replace the endogenous cleavage site (SEQ ID NO: 7) with cleavage site R10 (SEQ ID NO: 17).

SEQ ID NO: 63 is a modified PBS1 amino acid sequence; it is modified to replace the endogenous cleavage site (SEQ ID NO: 7) with cleavage site R11 (SEQ ID NO: 18).

SEQ ID NO: 64 is a modified PBS1 amino acid sequence; it is modified to replace the endogenous cleavage site (SEQ ID NO: 7) with cleavage site R12 (SEQ ID NO: 19).

SEQ ID NO: 65 is a modified PBS1 amino acid sequence; it is modified to replace the endogenous cleavage site (SEQ ID NO: 7) with cleavage site R13 (SEQ ID NO: 20).

SEQ ID NO: 66 is a modified PBS1 amino acid sequence; it is modified to replace the endogenous cleavage site (SEQ ID NO: 7) with cleavage site R14 (SEQ ID NO: 21).

SEQ ID NO: 67 is a modified PBS1 amino acid sequence; it is modified to replace the endogenous cleavage site (SEQ ID NO: 7) with cleavage site R15 (SEQ ID NO: 22).

SEQ ID NO: 68 is a modified PBS1 amino acid sequence; it is modified to replace the endogenous cleavage site (SEQ ID NO: 7) with cleavage site R16 (SEQ ID NO: 23).

SEQ ID NO: 69 is a modified PBS1 amino acid sequence; it is modified to replace the endogenous cleavage site (SEQ ID NO: 7) with cleavage site R17 (SEQ ID NO: 24).

SEQ ID NO: 70 is a modified PBS1 amino acid sequence; it is modified to replace the endogenous cleavage site (SEQ ID NO: 7) with cleavage site R18 (SEQ ID NO: 25).

SEQ ID NO: 71 is a modified PBS1 amino acid sequence; it is modified to replace the endogenous cleavage site (SEQ ID NO: 7) with cleavage site R19 (SEQ ID NO: 26).

SEQ ID NO: 72 is a modified PBS1 amino acid sequence; it is modified to replace the endogenous cleavage site (SEQ ID NO: 7) with cleavage site R20 (SEQ ID NO: 27).

SEQ ID NO: 73 is a modified PBS1 amino acid sequence; it is modified to replace the endogenous cleavage site (SEQ ID NO: 7) with cleavage site R21 (SEQ ID NO: 28).

SEQ ID NO: 74 is a modified PBS1 amino acid sequence; it is modified to replace the endogenous cleavage site (SEQ ID NO: 7) with cleavage site R22 (SEQ ID NO: 29).

SEQ ID NO: 75 is a modified PBS1 amino acid sequence; it is modified to replace the endogenous cleavage site (SEQ ID NO: 7) with cleavage site S1 (SEQ ID NO: 30).

SEQ ID NO: 76 is a modified PBS1 amino acid sequence; it is modified to replace the endogenous cleavage site (SEQ ID NO: 7) with cleavage site S2 (SEQ ID NO: 31).

SEQ ID NO: 77 is a modified PBS1 amino acid sequence; it is modified to replace the endogenous cleavage site (SEQ ID NO: 7) with cleavage site S3 (SEQ ID NO: 32).

SEQ ID NO: 78 is a modified PBS1 amino acid sequence; it is modified to replace the endogenous cleavage site (SEQ ID NO: 7) with cleavage site S4 (SEQ ID NO: 33).

SEQ ID NO: 79 is a modified PBS1 amino acid sequence; it is modified to replace the endogenous cleavage site (SEQ ID NO: 7) with cleavage site S5 (SEQ ID NO: 34).

SEQ ID NO: 80 is a modified PBS1 amino acid sequence; it is modified to replace the endogenous cleavage site (SEQ ID NO: 7) with cleavage site S6 (SEQ ID NO: 35).

SEQ ID NO: 81 is a modified PBS1 amino acid sequence; it is modified to replace the endogenous cleavage site (SEQ ID NO: 7) with cleavage site S7 (SEQ ID NO: 36).

SEQ ID NO: 82 is a modified PBS1 amino acid sequence; it is modified to replace the endogenous cleavage site (SEQ ID NO: 7) with cleavage site S8 (SEQ ID NO: 37).

SEQ ID NO: 83 is a modified PBS1 amino acid sequence; it is modified to replace the endogenous cleavage site (SEQ ID NO: 7) with cleavage site S9 (SEQ ID NO: 38).

SEQ ID NO: 84 is a modified PBS1 amino acid sequence; it is modified to replace the endogenous cleavage site (SEQ ID NO: 7) with cleavage site S10 (SEQ ID NO: 39).

SEQ ID NO: 85 is a modified PBS1 amino acid sequence; it is modified to replace the endogenous cleavage site (SEQ ID NO: 7) with cleavage site S11 (SEQ ID NO: 40).

SEQ ID NO: 86 is a modified PBS1 amino acid sequence; it is modified to replace the endogenous cleavage site (SEQ ID NO: 7) with cleavage site S12 (SEQ ID NO: 41).

SEQ ID NO: 87 is a modified PBS1 amino acid sequence; it is modified to replace the endogenous cleavage site (SEQ ID NO: 7) with cleavage site S13 (SEQ ID NO: 42).

SEQ ID NO: 88 is a modified PBS1 amino acid sequence; it is modified to replace the endogenous cleavage site (SEQ ID NO: 7) with cleavage site S14 (SEQ ID NO: 43).

SEQ ID NO: 89 is a modified PBS1 amino acid sequence; it is modified to replace the endogenous cleavage site (SEQ ID NO: 7) with cleavage site S15 (SEQ ID NO: 44).

SEQ ID NO: 90 is a modified PBS1 amino acid sequence; it is modified to replace the endogenous cleavage site (SEQ ID NO: 7) with cleavage site S16 (SEQ ID NO: 45).

SEQ ID NO: 91 is a modified PBS1 amino acid sequence; it is modified to replace the endogenous cleavage site (SEQ ID NO: 7) with cleavage site S17 (SEQ ID NO: 46).

SEQ ID NO: 92 is a modified PBS1 amino acid sequence; it is modified to replace the endogenous cleavage site (SEQ ID NO: 7) with cleavage site S18 (SEQ ID NO: 47).

SEQ ID NO: 93 is a modified PBS1 amino acid sequence; it is modified to replace the endogenous cleavage site (SEQ ID NO: 7) with cleavage site S20 (SEQ ID NO: 48).

SEQ ID NO: 94 is a modified PBS1 amino acid sequence; it is modified to replace the endogenous cleavage site (SEQ ID NO: 7) with cleavage site S21 (SEQ ID NO: 49).

SEQ ID NO: 95 is a modified PBS1 amino acid sequence; it is modified to replace the endogenous cleavage site (SEQ ID NO: 7) with cleavage site S23 (SEQ ID NO: 50).

SEQ ID NO: 96 is a modified PBS1 amino acid sequence; it is modified to replace the endogenous cleavage site (SEQ ID NO: 7) with cleavage site S24 (SEQ ID NO: 51).

SEQ ID NO: 97 is a modified PBS1 amino acid sequence; it is modified to replace the endogenous cleavage site (SEQ ID NO: 7) with cleavage site R26 (SEQ ID NO: 52).

SEQ ID NO: 98 is the amino acid sequence of the R2-S5 cleavage site peptide.

SEQ ID NO: 99 is the amino acid sequence of the R2-S6 cleavage site peptide.

SEQ ID NO: 100 is the amino acid sequence of the R2-S20 cleavage site peptide.

SEQ ID NO: 101 is the amino acid sequence of the R2-S21 cleavage site peptide.

SEQ ID NO: 102 is the amino acid sequence of the R2-S23 cleavage site peptide.

SEQ ID NO: 103 is the amino acid sequence of the R2-S24 cleavage site peptide.

SEQ ID NO: 104 is the amino acid sequence of the R10-S5 cleavage site peptide.

SEQ ID NO: 105 is the amino acid sequence of the R10-S6 cleavage site peptide.

SEQ ID NO: 106 is the amino acid sequence of the R10-S20 cleavage site peptide.

SEQ ID NO: 107 is the amino acid sequence of the R10-S21 cleavage site peptide.

SEQ ID NO: 108 is the amino acid sequence of the R10-S23 cleavage site peptide.

SEQ ID NO: 109 is the amino acid sequence of the R10-S24 cleavage site peptide.

SEQ ID NO: 110 is the amino acid sequence of the R14-S5 cleavage site peptide.

SEQ ID NO: 111 is the amino acid sequence of the R14-S6 cleavage site peptide.

SEQ ID NO: 112 is the amino acid sequence of the R14-S20 cleavage site peptide.

SEQ ID NO: 113 is the amino acid sequence of the R14-S21 cleavage site peptide.

SEQ ID NO: 114 is the amino acid sequence of the R14-S23 cleavage site peptide.

SEQ ID NO: 115 is the amino acid sequence of the R14-S24 cleavage site peptide.

SEQ ID NO: 116 is the amino acid sequence of the R17-S5 cleavage site peptide.

SEQ ID NO: 117 is the amino acid sequence of the R17-S6 cleavage site peptide.

SEQ ID NO: 118 is the amino acid sequence of the R17-S20 cleavage site peptide.

SEQ ID NO: 119 is the amino acid sequence of the R17-S21 cleavage site peptide.

SEQ ID NO: 120 is the amino acid sequence of the R17-S23 cleavage site peptide.

SEQ ID NO: 121 is the amino acid sequence of the R17-S24 cleavage site peptide.

SEQ ID NO: 122 is the amino acid sequence of the R19-S5 cleavage site peptide.

SEQ ID NO: 123 is the amino acid sequence of the R19-S6 cleavage site peptide.

SEQ ID NO: 124 is the amino acid sequence of the R19-S20 cleavage site peptide.

SEQ ID NO: 125 is the amino acid sequence of the R19-S21 cleavage site peptide.

SEQ ID NO: 126 is the amino acid sequence of the R19-S23 cleavage site peptide.

SEQ ID NO: 127 is the amino acid sequence of the R19-S24 cleavage site peptide.

SEQ ID NO: 128 is the amino acid sequence of the R20-S5 cleavage site peptide.

SEQ ID NO: 129 is the amino acid sequence of the R20-S6 cleavage site peptide.

SEQ ID NO: 130 is the amino acid sequence of the R20-S20 cleavage site peptide.

SEQ ID NO: 131 is the amino acid sequence of the R20-S21 cleavage site peptide.

SEQ ID NO: 132 is the amino acid sequence of the R20-S23 cleavage site peptide.

SEQ ID NO: 133 is the amino acid sequence of the R20-S24 cleavage site peptide.

SEQ ID NO: 134 is the amino acid sequence of the R21-S5 cleavage site peptide.

SEQ ID NO: 135 is the amino acid sequence of the R21-S6 cleavage site peptide.

SEQ ID NO: 136 is the amino acid sequence of the R21-S20 cleavage site peptide.

SEQ ID NO: 137 is the amino acid sequence of the R21-S21 cleavage site peptide.

SEQ ID NO: 138 is the amino acid sequence of the R21-S23 cleavage site peptide.

SEQ ID NO: 139 is the amino acid sequence of the R21-S24 cleavage site peptide.

SEQ ID NO: 140 is the amino acid sequence of the R22-S5 cleavage site peptide.

SEQ ID NO: 141 is the amino acid sequence of the R22-S6 cleavage site peptide.

SEQ ID NO: 142 is the amino acid sequence of the R22-S20 cleavage site peptide.

SEQ ID NO: 143 is the amino acid sequence of the R22-S21 cleavage site peptide.

SEQ ID NO: 144 is the amino acid sequence of the R22-S23 cleavage site peptide.

SEQ ID NO: 145 is the amino acid sequence of the R22-S24 cleavage site peptide.

SEQ ID NO: 146 is an amino acid motif for the soy rust cleavage site.

SEQ ID NO: 147 is an amino acid motif for the nematode cleavage site.

SEQ ID NO: 148 is an amino sequence of Arabidopsis thaliana RIN4.

SEQ ID NO: 149 is a nucleotide sequence of Arabidopsis thaliana RIN4.

SEQ ID NO: 150 is the amino acid sequence of glyma.Wm82.gnm4.ann1.Glyma.20G249600.1, a Glycine homolog of Arabidopsis thaliana PBS1.

SEQ ID NO: 151 is the amino acid sequence of glyma.Lee.gnm1.ann1.GlymaLee.20G209700.1, a Glycine homolog of Arabidopsis thaliana PBS1.

SEQ ID NO: 152 is the amino acid sequence of glyma.Zh13.gnm1.ann1.SoyZH13_20G232600.m1, a Glycine homolog of Arabidopsis thaliana PBS1.

SEQ ID NO: 153 is a nucleotide sequence of a gene encoding Arabidopsis PBS1 (SEQ ID NO: 1), including 5′- and 3′-UTRs.

### BRIEF DESCRIPTION OF THE FIGURES

FIG. 1 is leaf illustration of a few of the PBS1 variants showing cleavage site (P⁺) dependent cell death compared to a negative control (P⁻) in N. tabacum for the soy rust effector.

FIG. 2 illustrates broad spectrum soy rust resistance of 3 PBS1 variants in soybean. The figure is broken up in columns A-K and there are three rows of leaves, herein referred to as the top row (rust1), middle row (rust2), and bottom row (rust3). The three rows refer to three soy rust strains used during evaluation. Column A represents leaves from a positive control event with copy number 2. Column B represents leaves with an event containing 1 copy of a PBS1 variant (construct 25327; PBS1-R19) and 1 copy of RPS5. Column C represents leaves with an event containing >2 copies of a PBS1 variant (construct 25327; PBS1-R19) and 0 copies of RPS5. Column D represents leaves from a soybean control. Column E represents leaves with an event containing 1 copy of a PBS1 variant (construct 25326; PBS-R17) and 1 copy of RPS5. Column F represents leaves with an event containing >2 copies of a PBS1 variant (construct 25326; PBS1-R17) and 1 copy of RPS5. Column G represents leaves with an event containing >2 copies of a PBS1 variant (construct 25326; PBS1-R17) and 0 copies of RPS5. Column H represents leaves from the soybean control. Column I represents leaves with an event containing >2 copies of a PBS1 variant (construct 25328; PBS1-R20) and 2 copies of RPS5. Column J represents leaves with an event containing 0 copies of a PBS1 variant (construct 25328; PBS1-R20) and >2 copies of RPS5. Column K represents leaves with an event containing >2 copies of a PBS1 variant (construct 25328; PBS1-R20) and 2 copies of RPS5.

FIG. 3 represents an example of a binary vector (25326) representing the R17 cleavage site peptide (SEQ ID NO: 24). Component “cAtPBS1-04” represents a modified version of Arabidopsis thaliana PBS1 (AvrPphB susceptible 1) gene with mutations in six amino acids from wild type (DKSHVS) to R17 (QVFEFL), encoding a Ser/Thr protein kinase.

FIG. 4 is leaf illustration of a few of the PBS1 variants showing cleavage site (P⁺) dependent cell death compared to a negative control (P⁻) in N. tabacum for the nematode effector.

FIG. 5 is a schematic representation of a method and system of modifying an endogenous cleavage site of a substrate protein of a plant to a heterogeneous cleavage site specific for a target plant pathogen to thereby confer the plant with resistance to the target plant pathogen. The unmodified substrate protein with the endogenous cleavage site is recognized by the plant's natural pathogen (Pathogen A in this example), resulting in an innate immune reaction due to interaction of the cleaved substrate protein with the R protein of the natural pathogen. The unmodified substrate protein, however, is not recognized by Pathogen B, resulting in no innate immune reaction. As a result, the plant is susceptible to infection by Pathogen B. Upon engineering the substrate protein to insert a heterogeneous cleavage sequence specific to Pathogen B, the modified substrate protein is recognized and cleaved, resulting in an innate immune reaction to pathogen B. This renders the plant resistant to Pathogen B.

FIG. 6 is a schematic representation of a modified PBS1 construct (SEQ ID NO: 53) illustrating the modification of the endogenous cleavage site (SEQ ID NO: 7) with an R17 cleavage site peptide (SEQ ID NO: 24) as disclosed in Example 4.

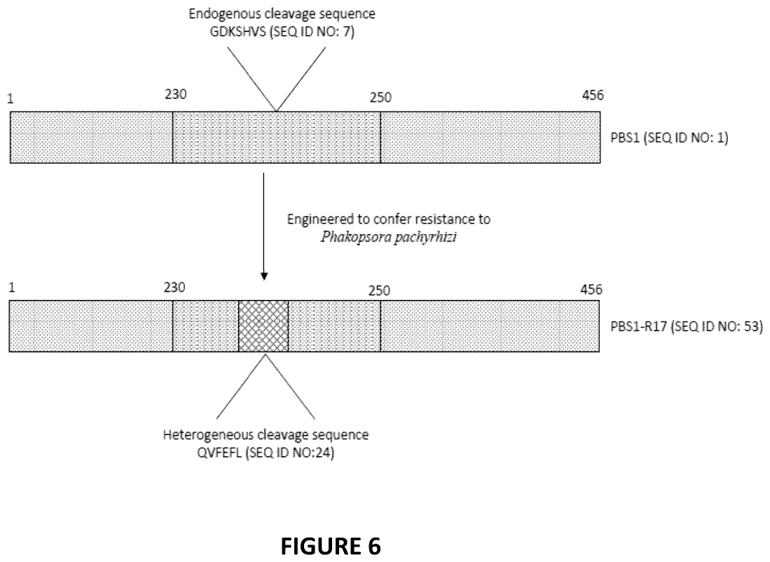

### DETAILED DESCRIPTION

All technical and scientific terms used herein, unless otherwise defined, are intended to have the same meaning as commonly understood by one of ordinary skill in the art. References to techniques employed herein are intended to refer to the techniques as commonly understood in the art, including variations on those techniques and/or substitutions of equivalent techniques that would be apparent to one of skill in the art.

Although the following terms are believed to be well understood by one of ordinary skill in the art, the following definitions are set forth to facilitate understanding of the presently disclosed subject matter.

As used in the description of the invention and the appended claims, the singular forms “a,” “an” and “the” are intended to include the plural forms as well, unless the context clearly indicates otherwise.

As used herein, “and/or” refers to and encompasses any and all possible combinations of one or more of the associated listed items, as well as the lack of combinations when interpreted in the alternative (“or”).

The term “about,” as used herein when referring to a measurable value such as a dosage or time period and the like, is meant to encompass variations of ±20%, ±10%, ±5%, ±1%, ±0.5%, or even ±0.1% of the specified amount. As used herein, phrases such as “between about X and Y” mean “between about X and about Y” and phrases such as “from about X to Y” mean “from about X to about Y.”

As used herein, phrases such as “between about X and Y”, “between about X and about Y”, “from X to Y” and “from about X to about Y” (and similar phrases) should be interpreted to include X and Y, unless the context indicates otherwise.

As used herein, “Basidiomycetes” or “Basidiomycete plant pathogen species” refers to plant pathogens belonging to the taxonomical divisional Basidiomycota. Plant pathogens included in this taxonomical classification includes members of the Phakopsora species, such as Phakopsora pachyrhizi responsible for the plant disease Asian Soy Rust (ASR).

As used herein, a “coding sequence” or “CDS” is a nucleic acid sequence that is transcribed into RNA such as mRNA, rRNA, tRNA, snRNA, sense RNA or antisense RNA. In embodiments, the RNA is then translated to produce a protein. In example embodiments, the CDS is derived from a cDNA sequence and includes the sequence of spliced exons of a transcript in DNA notation and does not include any intron or 5′ or 3′-untranslated regions (UTRs). In comparison, the cDNA sequence contains the whole sequence of the corresponding RNA in DNA notation, including coding and untranslated sequences.

As used herein, a “codon optimized” nucleotide sequence means a nucleotide sequence of a recombinant, transgenic, or synthetic polynucleotide wherein the codons are chosen to reflect the particular codon bias that a host cell or organism may have. This is typically done in such a way so as to preserve the amino acid sequence of the polypeptide encoded by the codon optimized nucleotide sequence. In certain embodiments, a nucleotide sequence is codon optimized for the cell (e.g., an animal, plant, fungal or bacterial cell) in which the construct is to be expressed. For example, a construct to be expressed in a plant cell can have all or parts of its sequence codon optimized for expression in a plant. See, for example, U.S. Pat. No. 6,121,014. In embodiments, the polynucleotides of the invention are codon-optimized for expression in a plant cell (e.g., a dicot cell or a monocot cell) or bacterial cell.

The term “comprise”, “comprises” or “comprising,” when used in this specification, indicates the presence of the stated features, integers, steps, operations, elements, or components, but does not preclude the presence or addition of one or more other features, integers, steps, operations, elements, components, and/or groups thereof.

As used herein, the transitional phrase “consisting essentially of” (and grammatical variants) means that the scope of a claim is to be interpreted to encompass the specified materials or steps recited in the claim “and those that do not materially alter the basic and novel characteristic(s)” of the claimed invention. Thus, the term “consisting essentially of” when used in a claim of this invention is not intended to be interpreted to be equivalent to “comprising.”

The term “corresponding to” in the context of nucleic acid sequences or protein sequences means that when the nucleic acid sequences or amino acid sequences of certain sequences are aligned with each other, the nucleic acids or amino acids that “correspond to” certain enumerated positions in the present invention are those that align with these positions in a reference sequence, but that are not necessarily in these exact numerical positions relative to a particular nucleic acid sequence of the invention. Optimal alignment of sequences for comparison can be conducted by computerized implementations of known algorithms or by visual inspection. Readily available sequence comparison and multiple sequence alignment algorithms are, respectively, the Basic Local Alignment Search Tool (BLAST) and ClustalW/ClustalW2/Clustal Omega programs available on the Internet (e.g., the website of the EMBL-EBI). Other suitable programs include, but are not limited to, GAP, BestFit, Plot Similarity, and FASTA, which are part of the Accelrys GCG Package available from Accelrys, Inc. of San Diego, Calif., United States of America. See also Smith & Waterman, 1981; Needleman & Wunsch, 1970; Pearson & Lipman, 1988; Ausubel et al., 1988; and Sambrook & Russell, 2001.

Unless otherwise stated, identity and similarity will be calculated by the Needleman-Wunsch global alignment and scoring algorithms (Needleman and Wunsch (1970) J. Mol. Biol. 48(3):443-453) as implemented by the “needle” program, distributed as part of the EMBOSS software package (Rice, P. Longden, and Bleasby, A., EMBOSS: The European Molecular Biology Open Software Suite, 2000, Trends in Genetics 16, (6) pp 276-277, versions 6.3.1 available from EMBnet at embnet.org/resource/emboss and emboss.sourceforge.net, among other sources) using default gap penalties and scoring matrices (EBLOSUM62 for protein and EDNAFULL for DNA). Equivalent programs may also be used. By “equivalent program” is intended any sequence comparison program that, for any two sequences in question, generates an alignment having identical nucleotide residue matches and an identical percent sequence identity when compared to the corresponding alignment generated by needle from EMBOSS version 6.3.1.

Additional mathematical algorithms are known in the art and can be utilized for the comparison of two sequences. See, for example, the algorithm of Karlin and Altschul (1990) Proc. Natl. Acad. Sci. USA 87:2264, modified as in Karlin and Altschul (1993) Proc. Natl. Acad. Sci. USA 90:5873-5877. Such an algorithm is incorporated into the BLAST programs of Altschul et al. (1990) J. Mol. Biol. 215:403. BLAST nucleotide searches can be performed with the BLASTN program (nucleotide query searched against nucleotide sequences) to obtain nucleotide sequences homologous to nucleic acid molecules of the invention, or with the BLASTX program (translated nucleotide query searched against protein sequences) to obtain protein sequences homologous to nucleic acid molecules of the invention. BLAST protein searches can be performed with the BLASTP program (protein query searched against protein sequences) to obtain amino acid sequences homologous to protein molecules of the invention, or with the TBLASTN program (protein query searched against translated nucleotide sequences) to obtain nucleotide sequences homologous to protein molecules of the invention. To obtain gapped alignments for comparison purposes, Gapped BLAST (in BLAST 2.0) can be utilized as described in Altschul et al. (1997) Nucleic Acids Res. 25:3389. Alternatively, PSI-Blast can be used to perform an iterated search that detects distant relationships between molecules. See Altschul et al. (1997) supra. When utilizing BLAST, Gapped BLAST, and PSI-Blast programs, the default parameters of the respective programs (e.g., BLASTX and BLASTN) can be used. Alignment may also be performed manually by inspection.

“Expression cassette” as used herein means a nucleic acid molecule capable of directing expression of at least one polynucleotide of interest, such as a polynucleotide encoding a modified substrate protein comprising a heterogenous cleavage site (such as a modified PBS1 or RIN4 comprising a heterogenous cleavage site), in an appropriate host cell, comprising a promoter operably linked to the polynucleotide of interest which is operably linked to a termination signal. The cassette will include 5′ and 3′ regulatory sequences operably linked to a polynucleotide encoding a polypeptide provided herein that allows for expression of the polynucleotide. The expression cassette may also comprise other polynucleotides not related to the expression of a polynucleotide of interest, but which are present to provide convenient restriction sites for removal of the cassette from an expression vector. In embodiments, at least one of the components in the expression cassette may be heterologous (i.e., foreign) with respect to at least one of the other components (e.g., a heterologous promoter operatively associated with a polynucleotide of interest). The expression cassette may also be one that is naturally occurring but has been obtained in a recombinant form useful for heterologous expression. Typically, however, the expression cassette is heterologous with respect to the host, i.e., the expression cassette (or even the polynucleotide of interest) does not occur naturally in the host cell and has been introduced into the host cell or an ancestor cell thereof by a transformation process or a breeding process. The expression of the polynucleotide(s) of interest in the expression cassette is generally under the control of a promoter. In the case of a multicellular organism, such as a plant, the promoter can also be specific or preferential to a particular tissue, or organ, or stage of development (as described in more detail herein). An expression cassette, or fragment thereof, can also be referred to as “inserted polynucleotide” or “insertion polynucleotide” when transformed into a plant. The cassette may additionally contain at least one additional gene or genetic element to be co-transformed into the organism. Where additional genes or elements are included, the components are operably linked. Alternatively, the additional gene(s) or element(s) can be provided on multiple expression cassettes.

As used herein, the terms “enhanced plant pathogen resistance”, “enhanced disease resistance”, and “conferring or enhancing resistance to a plant pathogen” refers to an improvement, enhancement, or increase in a plant's ability to endure and/or thrive despite being infected with a target plant pathogen (such as a plant pathogen against which the plant does not have innate immunity) as compared to one or more control plants. An enhanced plant pathogen resistance comprises any statistically significant increase in resistance to the plant pathogen, including, for example, an increase of at least 1%, 5%, 10%, 20%, 30%, 40%, 50%, 60%, 70%, 80%, 90%, 100% or higher. The control plant may be a plant expressing an endogenous substrate protein with an unmodified cleavage site, wherein the unmodified cleavage site is not recognized or cleaved by a protease of the target pathogen. As a result, a strong immune response is not elicited in the plant responsive to infection by the target pathogen (e.g., no immune response is triggered or a weak immune response is triggered). Such a control plant is fully susceptible to the pathogen or may have limited resistance to the pathogen. The plant with the enhanced disease resistance expresses a modified substrate protein (e.g., modified PBS1 or modified RIN4) wherein the endogenous cleavage site is engineered, via the inclusion of one or more selective mutations, to a heterogenous cleavage site that is recognized and cleaved by a protease of the target pathogen. As a result of expression of the modified substrate protein comprising the heterogenous cleavage site, an immune response is triggered in the plant that reduces the expression of symptoms indicative of infection by the target pathogen. In example embodiments, the substrate protein is engineered to include the heterologous cleavage site for a Basidiomycete species specific plant pathogen, such as a Phytophthora species plant pathogen (e.g., the pathogen responsible for causing Asian soybean rust) or a Nematoda species plant pathogen, such as a Heterodera species plant pathogen (such as the pathogen responsible for causing soybean cysts).

Conferring or enhancing of resistance against a plant pathogen may include an increase (partial or complete increase) in phenotypic characteristics associated with plant pathogen specific protease dependent cell death, herein also referred to as a “hypersensitive response”. In one embodiment, enhanced resistance is associated with a greater degree of hypersensitive response, including but not limited to increased electrolyte leakage from a site of infection.

As used herein, the term “endogenous” refers to materials originating from within an organism or cell. In contrast, “heterogenous” or “heterologous” refers to materials not originating naturally from within the organism or cell, due to modifications being artificially introduced to their endogenous state. For example, a modified plant HRS protein of the present invention comprising a cleavage peptide sequence that is modified (via one or more amino acid substitutions) from the endogenous sequence so as to be recognized by a target pathogen specific protease is a modified HRS protein with a heterogenous sequence. As such, the unmodified protein, with the endogenous cleavage peptide sequence, would not be recognized by the target pathogen and would not trigger a corresponding immune response in the plant.

The term “gene” or “genomic sequence” means a nucleic acid that comprises chromosomal DNA, genomic DNA, plasmid DNA, cDNA, an artificial DNA polynucleotide, or other DNA encoding a polypeptide of interest. In particular embodiments, the nucleic acid sequence of the gene encodes a protein that, when expressed, is responsible, at least in part, for a particular characteristic or trait. In embodiments, the gene may be native, modified (e.g., by directed recombination or site-specific mutation), or synthetic. In example embodiments, the gene is transcribed into an RNA molecule (e.g., an mRNA) in a cell wherein the RNA may encode a peptide, polypeptide, or protein of interest, and in some examples may also encode genetic elements flanking the coding sequence that are involved in the regulation of expression of the mRNA or polypeptide of the present invention. A gene may thus comprise several operably linked sequences, such as a promoter sequence, a 5′ leader sequence comprising, for example, sequences involved in translation initiation, a (protein) coding region (comprising cDNA or genomic DNA), a 3′ non-translated sequence comprising, for example, transcription termination sequence sites, introns (e.g., one or more native, foreign, or modified introns). In example embodiments, the nucleic acid sequence of the isolated gene may include introns, exons, 5′ or 3′-untranslated regions (UTRs), and native regulatory elements (such as native promoters). In other example embodiments, the gene comprises a coding sequence for a polypeptide of interest without including any regulatory elements (e.g., without any native or foreign introns, with some native introns replaced with foreign or modified introns, without any untranslated sequences, or with native regulatory elements replaced with foreign, heterologous or modified regulatory elements).

As used herein, in particular embodiments, “R-gene” or “Resistance gene” refers to a nucleic acid (e.g., DNA sequence) encoding a R-protein, or Resistance protein, that when expressed in a plant cell, can confer to the plant cell, and/or the plant comprising the plant cell, increased resistance to one or more plant pathogens. In embodiments, the R-gene may comprise one or more motifs that correlate with one or more domains of the corresponding R-protein. For example, embodiments of the R-gene may comprise a TNL motif comprising a Toll/Interleukin-1 receptor (TIR) motif, a nucleotide-binding site (NBS), and a leucine rich-repeat (LRR) motif. When expressed, the TNL motif encodes a TNL motif in the R-protein comprising a Toll/Interleukin-1 receptor (TIR) domain, a nucleotide-binding site (NBS) domain, and a leucine rich-repeat (LRR) domain. Examnple R-genes, and R proteins encoded by corresponding R-genes including RPS1-5 are known (see for example, Warren et al. Genetics (1999), U.S. Pat. No. 7,696,410, WO2019182884A1, etc., all of which are incorporated by reference herein). As used herein, “recombinant” refers to a form of nucleic acid (e.g., DNA or RNA) and/or protein and/or an organism that would not normally be found in nature and as such was created by human intervention. Such human intervention may produce a recombinant nucleic acid molecule and/or a recombinant plant. As used herein, a “recombinant DNA molecule” is a DNA molecule comprising a combination of DNA molecules that would not naturally occur together and is the result of human intervention, e.g., a DNA molecule that is comprised of a combination of at least two DNA molecules heterologous to each other, and/or a DNA molecule that is artificially synthesized and comprises a polynucleotide that deviates from the polynucleotide that would normally exist in nature, and/or a DNA molecule that is artificially incorporated into a host cell's genomic DNA and the associated flanking DNA of the host cell's genome. An example of a recombinant DNA molecule is a DNA molecule resulting from the insertion of the transgene or a genome modification (i.e., a gene edit) into a plant's genomic DNA, which may ultimately result in the expression of a recombinant RNA and/or protein molecule in that organism. As used herein, a “recombinant plant” is a plant that would not normally exist in nature, is the result of human intervention, and contains a transgene and/or heterologous DNA molecule and/or a genome modification (i.e., a gene edit) incorporated into its genome. As a result of such genomic alteration, the recombinant plant is distinctly different from the related wildtype plant.

An example of a recombinant nucleic acid molecule encoding a modified substrate protein of a pathogen-specific protease includes a nucleotide sequence that encodes PBS1 in which its endogenous cleavage site (SEQ ID NO: 7) is replaced with a heterologous cleavage site (SEQ ID NO: 24), as is shown in SEQ ID NO: 69. Another example of a recombinant nucleic acid molecule encoding a modified substrate protein of a pathogen-specific protease includes a nucleotide sequence that encodes RIN4 in which its endogenous cleavage site is replaced with a heterologous TEV protease cleavage site.

For nucleotide sequences, “variant” means a substantially similar nucleotide sequence to a nucleotide sequence of a recombinant nucleic acid molecule as described herein, for example, a substantially similar nucleotide sequence encoding a modified substrate protein. For nucleotide sequences, a variant comprises a nucleotide sequence having deletions (i.e., truncations) at the 5′ and/or 3′ end, deletions and/or additions of one or more nucleotides at one or more internal sites compared to the nucleotide sequence of the recombinant nucleic acid molecules as described herein; and/or substitution of one or more nucleotides at one or more sites compared to the nucleotide sequence of the recombinant nucleic acid molecules described herein. One of skill in the art understands that variants are constructed in a manner to maintain the open reading frame.

Conservative variants include those nucleotide sequences that, because of the degeneracy of the genetic code, result in a functionally active modified substrate protein as described herein. Naturally occurring allelic variants can be identified by using well-known molecular biology techniques such as, for example, polymerase chain reaction (PCR) and hybridization techniques. Variant nucleotide sequences also can include synthetically derived sequences, such as those generated, for example, by site-directed mutagenesis but which still provide a functionally active modified substrate protein. Generally, variants of a nucleotide sequence of the recombinant nucleic acid molecules as described herein will have at least about 70%, 75%, 80%, 85%, 90%, 91%, 92%, 93%, 94%, 95%, 96%, 97%, 98%, 99% or more sequence identity to the nucleotide sequence of the recombinant nucleic acid molecules as determined by sequence alignment programs and parameters as described elsewhere herein.

As used herein, “nucleic acid”, “nucleic acid molecule”, or “polynucleotide” refers to any physical string of monomer units that can be corresponded to a string of nucleotides, including a polymer of nucleotides (e.g., a typical DNA polymer or polydeoxyribonucleotide or RNA polymer or polyribonucleotide), modified oligonucleotides (e.g., oligonucleotides comprising bases that are not typical to biological RNA or DNA, such as 2′-O-methylated oligonucleotides), and the like. In some embodiments, a nucleic acid or polynucleotide can be single-stranded, double-stranded, multi-stranded, or combinations thereof. Unless otherwise indicated, a particular nucleic acid or polynucleotide of the present invention optionally comprises or encodes complementary polynucleotides, in addition to any polynucleotide explicitly indicated.

As used herein, “promoter” refers to refers to a polynucleotide, usually upstream (5′) of its coding polynucleotide in an expression cassette, which controls the expression of the coding polynucleotide by providing the recognition for RNA polymerase and other factors required for proper transcription. The promoter is a regulatory element capable of binding RNA polymerase and initiating transcription of a downstream (3′-direction) coding sequence. A number of promoters can be used in an expression cassette, including the native promoter of the gene encoding an HRS protein or the native promoter of an R gene encoding an R protein.

Alternatively, promoters can be selected based upon a desired outcome. Such promoters include, but are not limited to, “constitutive promoters” (where expression of a polynucleotide sequence operably linked to the promoter is unregulated and therefore continuous), “inducible promoters” (where expression of a polynucleotide sequence operably linked to the promoter is induced by an analyte, cofactor, regulatory protein, etc.), “repressible promoters” (where expression of a polynucleotide sequence operably linked to the promoter is repressed by an analyte, cofactor, regulatory protein, etc.), and “tissue-preferred promoters” (where expression of a polynucleotide sequence operably linked to the promoter is higher in a preferred tissue relative to other tissues, such as higher in leaf tissue relative to other plant tissues).

As used herein, “plant promoter” means a promoter that drives expression in a plant such as a constitutive, inducible (e.g., chemical-, environmental-, pathogen- or wound-inducible), repressible, tissue-preferred or other promoter for use in plants.

Example promoters are set forth in WO 99/43838 and in U.S. Pat. Nos. 8,575,425; 7,790,846; 8,147,856; 8,586,832; 7,772,369; 7,534,939; 6,072,050; 5,659,026; 5,608,149; 5,608,144; 5,604,121; 5,569,597; 5,466,785; 5,399,680; 5,268,463; 5,608,142; and 6,177,611; herein incorporated by reference. Example constitutive promoters include CaMV 35S promoter (Odell et al. (985) Nature 313:810-812); rice actin (McElroy et al. (1990) Plant Cell 2: 163-171); ubiquitin (Christensen et al. (1989) Plant Mol. Biol. 12:619-632 and Christensen et al. (1992) Plant Mol. Biol. 18:675-689); pEMU (Last et al. (1991) Theor. Appl. Genet. 81: 581-588); MAS (Velten et al. (1984) EMBO J. 3:2723-2730). Example inducible promoters include those that drive expression of pathogenesis-related proteins (PR proteins), which are induced following infection by a pathogen. See, for example, Redolfi et al. (1983) Neth. J. Plant Pathol. 89:245-254; Uknes et al. (1992) Plant Cell 4:645-656; and Van Loon (1985) Plant Mol. Virol. 4: 111-116; and WO 99/43819, herein incorporated by reference. Promoters that are expressed locally at or near the site of pathogen infection may also be used (Marineau et al. (1987) Plant Mol. Biol. 9:335-342; Matton et al. (1989) Molecular Plant-Microbe Interactions 2: 325-331; Somsisch et al. (1986) Proc. Natl. Acad. Sci. USA 83:2427-2430; Somsisch et al. (1988) Mol. Gen. Genet. 2:93-98; and Yang (1996) Proc. Natl. Acad. Sci. USA 93: 14972-14977; Chen et al. (1996) Plant J. 10:955-966; Zhang et al. (1994) Proc. Natl. Acad. Sci. USA 91:2507-2511; Warner et al. (1993) Plant J. 3: 191-201; Siebertz et al. (1989) Plant Cell 1:961-968; Cordero et al. (1992) Physiol. Mol. Plant Path. 41: 189-200; U.S. Pat. No. 5,750,386 (nematode-inducible); and the references cited therein).

Wound-inducible promoters include pin II promoter (Ryan (1990) Ann. Rev. Phytopath. 28:425-449; Ouan et al. (1996) Nature Biotechnology 14:494-498); wun1 and wun2 (U.S. Pat. No. 5,428,148); win1 and win2 (Stanford et al. (1989) Mol. Gen. Genet. 215:200-208); systemin (McGurl et al. (1992) Science 225: 1570-1573); WIP1 (Rohmeier et al. (1993) Plant Mol. Biol. 22:783-792; Eckelkamp et al. (1993) FEBS Letters 323:73-76); MPI gene (Corderok et al. (1994) Plant J. 6(2): 141-150); and the like, herein incorporated by reference.

Tissue-preferred promoters for use in the invention include those set forth in Yamamoto et al. (1997) Plant J. 12(2):255-265; Kawamata et al. (1997) Plant Cell Physiol. 38(7):792-803; Hansen et al. (1997) Mol. Gen Genet. 254(3):337-343; Russell et al. (1997) Transgenic Res. 6(2): 157-168; Rinehart et al. (1996) Plant Physiol. 112(3): 1331-1341; Van Camp et al. (1996) Plant Physiol. 112(2):525-535; Canevascim et al. (1996) Plant Physiol. 112(2):513-524; Yamamoto et al. (1994) Plant Cell Physiol. 35(5):773-778; Lam (1994) Results Probl. Cell Differ. 20: 181-196; Orozco et al. (1993) PlantMolBiol. 23(6): 1129-1138; Matsuoka et al. (1993) Proc Natl. Acad. Sci. USA 90(20):9586-9590; and Guevara-Garcia et al. (1993) Plant J. 4(3):495-505.

Leaf-preferred promoters include those set forth in Yamamoto et al. (1997) Plant J. 12(2):255-265; Kwon et al. (1994) Plant Physiol. 105:357-67; Yamamoto et al. (1994) Plant Cell Physiol. 35(5):773-778; Gotor et al. (1993) Plant J. 3:509-18; Orozco et al. (1993) Plant Mol. Biol. 23(6): 1129-1138; and Matsuoka et al. (1993) Proc. Natl. Acad. Sci. USA 90(20):9586-9590.

Root-preferred promoters are known and include those in Hire et al. (1992) Plant Mol. Biol. 20(2):207-218 (soybean root-specific glutamine synthetase gene); Keller and Baumgartner (1991) Plant Cell 3(10): 1051-1061 (root-specific control element); Sanger et al. (1990) Plant Mol. Biol. 14(3):433-443 (mannopine synthase (MAS) gene of Agrobacterium tumefaciens); and Miao et al. (1991) Plant Cell 3(1): 11-22 (cytosolic glutamine synthetase (GS)); Bogusz et al. (1990) Plant Cell 2(7):633-641; Leach and Aoyagi (1991) Plant Science (Limerick) 79(1):69-76 (rolC and rolD); Teeri et al. (1989) EMBO J. 8(2):343-350; Kuster et al. (1995) Plant Mol. Biol. 29(4):759-772 (the VfENOD-GRP3 gene promoter); and, Capana et al. (1994) Plant Mol. Biol. 25(4):681-691 (rolB promoter). See also U.S. Pat. Nos. 5,837,876; 5,750,386; 5,633,363; 5,459,252; 5,401,836; 5,110,732; and 5,023,179.

As used herein, “operably linked” refers to the association of polynucleotides on a single nucleic acid fragment so that the function of one affects the function of the other. For example, a promoter is operably linked with a coding polynucleotide or functional RNA when it is capable of affecting the expression of that coding polynucleotide or functional RNA (i.e., that the coding polynucleotide or functional RNA is under the transcriptional control of the promoter). Coding polynucleotide in sense or antisense orientation can be operably linked to regulatory polynucleotides.

As used herein, “protein” refers to a polynucleotide, usually upstream (5′) of its coding polynucleotide, which controls the expression of the coding polynucleotide by providing the recognition for RNA polymerase and other factors required for proper transcription.

As used herein, “substrate protein” refers to a molecule upon which an enzyme reacts. In the present invention, for example, the substrate protein includes hypersensitive response substrate (HRS) proteins such as PBS1 and RIN4, which are recognized and cleaved by pathogen specific proteases resulting in a hypersensitive defense response in the plant against the pathogen. In particular, the pathogen specific protease, upon recognition of a pathogen specific cleavage sequence in the substrate protein, binds and cleaves the substrate protein at the cleavage sequence. The cleaved substrate protein is then able to interact with a resistance protein of the plant, triggering the hypersensitive response.

As used herein, “protease” refers to an enzyme which breaks down proteins and peptides.

As used herein, “plant pathogen” refers to any organism that causes disease on plants. Examples of plant pathogens include, but are not limited to, viruses, fungi, bacteria, and nematodes.

As used herein, “hypersensitive response substrate protein (HRS)” refers to a substrate protein, as described above, in which activity upon the protein results in a hypersensitive response in a plant.

As used herein, “hypersensitive response” refers to a rapid localized immune response of a plant to a pathogen at the point of entry of the pathogen in the plant. The response is used to prevent further spread of said pathogen infection and results in a quick death of the cells in the localized area.

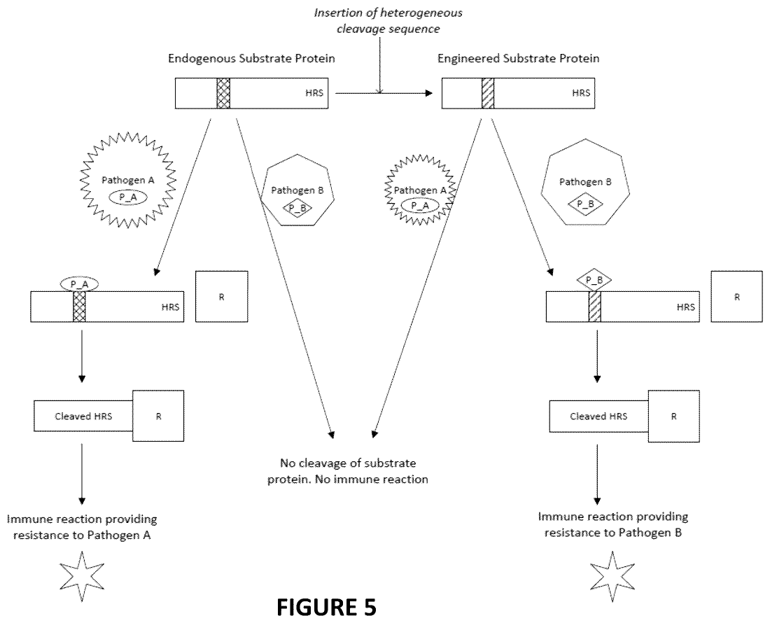

As used herein, “PBS1” refers to a protein kinase superfamily protein involved in plant defense and has been previously disclosed, for example, in U.S. Pat. No. 9,816,102. The defense mechanism may be mediated by the disease resistance (R) protein RPS5. As understood by those skilled in the art, “PBS1” refers to avrPphB susceptible 1. By “PBS1 activity” is intended a polypeptide that when recognized and cleaved by a pathogen-specific protease, mounts a hypersensitive defense response in the plant against the pathogen. In particular, the pathogen specific protease, upon recognition of a pathogen specific cleavage sequence in the PBS1 polypeptide, binds and cleaves the PBS1 polypeptide at the cleavage sequence. The cleaved PBS1 substrate protein is then able to interact with a resistance protein of the plant, triggering the hypersensitive response (FIG. 5 shows a simplified example of such an interaction between a substrate protein and a pathogen-specific protease).

As used herein, “RIN4” refers to, like PBS1, a regulator of plant defense. As understood by those skilled in the art, “RIN4” refers to Resistance to Pseudomonas syringae pv. maculicola 1 (“RPM1”) Interacting Protein 4. By “RIN4 activity” is intended a polypeptide that is recognized and cleaved by pathogen specific proteases, a hypersensitive defense response in the plant against the pathogen occurs. In particular, the pathogen specific protease, upon recognition of a pathogen specific cleavage sequence in the RIN4 polypeptide, binds and cleaves the RIN4 polypeptide at the cleavage sequence. The cleaved RIN4 polypeptide is then able to interact with a resistance protein of the plant, triggering the hypersensitive response.

As used herein, “RPS5” refers to a disease resistance protein encoded by the Rps5 gene. Interaction of the resistance protein with a substrate protein fragment, cleaved by a pathogen protease, enables the plant expressing the resistance protein to mount an innate immune reaction that confers the plant with resistance to the specific plant pathogen.

As used herein, “heterologous” refers to, when used in reference to a gene or nucleic acid, a gene encoding a factor that is not in its natural environment (i.e., has been altered by the hand of man). For example, a heterologous gene may include a gene from one species introduced into another species. A heterologous gene may also include a gene native to an organism that has been altered in some way (e.g., mutated, added in multiple copies, linked to a non-native promoter or enhancer polynucleotide, etc.). Heterologous genes further may comprise plant gene polynucleotides that comprise cDNA forms of a plant gene; the cDNAs may be expressed in either a sense (to produce mRNA) or anti-sense orientation (to produce an anti-sense RNA transcript that is complementary to the mRNA transcript). In one aspect of the invention, heterologous genes are distinguished from endogenous plant genes in that the heterologous gene polynucleotide are typically joined to polynucleotides comprising regulatory elements such as promoters that are not found naturally associated with the gene for the protein encoded by the heterologous gene or with plant gene polynucleotide in the chromosome, or are associated with portions of the chromosome not found in nature (e.g., genes expressed in loci where the gene is not normally expressed). Further, in embodiments, a “heterologous” polynucleotide is a polynucleotide not naturally associated with a host cell into which it is introduced, including non-naturally occurring multiple copies of a naturally occurring polynucleotide.

As used herein, “cleavage site” refers to a specific peptide sequence (or motif) that can be cleaved or cut by a site-specific protease. The protease may be pathogen specific. A given protease derived from a specific pathogen recognizes a specific cleavage site sequence, or a variant thereof.

A “Basidiomycete-specific cleavage site” or “Basidiomycete-specific cleavage sequence” refers to a cleavage site or amino acid sequence that is specifically recognized by a Basidiomycete plant pathogen, such as a Phakopsora plant pathogen including but not limited to Phakopsora pachyrhizi responsible for causing Asian soybean rust (ASR). In embodiments where the “Basidiomycete-specific cleavage sequence” is specific for Phakopsora pachyrhizi and/or is a cleavage sequence being assessed for conferring resistance to Asian soy rust, the cleavage sequence/peptide may also be referred to herein as a “Soy rust cleavage peptide”.

A “Nematode-specific cleavage site” or “Neamtode-specific cleavage sequence” refers to a cleavage site or amino acid sequence that is specifically recognized by a Nematode plant pathogen, such as a Heterodera plant pathogen including but not limited to Heterodera glycines responsible for causing Soybean cysts (also referred to Soybean Cyst Nematode or SCN). In embodiments where the “Nematode-specific cleavage sequence” is specific for Heterodera glycines and/or is a cleavage sequence being assessed for conferring resistance to SCN, the cleavage sequence/peptide may also be referred to herein as a “Soybean cyst cleavage peptide”.

As used herein, the terms “confer pathogen resistance”, “confer disease resistance”, and “conferring or enhancing resistance to a pathogen” refers to an improvement, enhancement, or increase in a plant's ability to endure and/or thrive despite being infected with a plant pathogen against which the plant does not have innate immunity (e.g., a Basidiomycete species pathogen, such as a Phakopsora plant pathogen including but not limited to Phakopsora pachyrhizi responsible for causing Asian soybean rust) as compared to one or more control plants (e.g., a plant comprising an endogenous substrate protein that has not been modified to comprise the cleavage site for a heterogenous R-gene or marker associated with enhanced pathogen resistance to respective pathogen/disease). The control plants may be fully susceptible to the pathogen or have limited resistance to the pathogen. Enhanced disease resistance includes any mechanism (other than whole-plant immunity or resistance) that reduces the expression of symptoms indicative of infection for a respective disease such as Asian soybean rust, soybean cyst nematode, Phytophthora, etc. Conferring or enhancing of resistance may include a reduction (partial reduction or complete reduction) in symptoms or phenotypic characteristics associated with susceptibility to the pathogen and/or an increase in phenotypic characteristics associated with resistance to the pathogen. In example embodiments, conferring or increasing of resistance to Asian Soy Rust can include an increase in the hypersensitive response in a plant following infection with a pathogen causing Asian Soy Rust.

As used herein, “improved resistance” refers to plants exhibiting greater resistance to a pathogen as compared to a control plant.

As used herein, “vector” refers to an agent that contains and carries modified genetic material. For example, a vector can be a plasmid or a virus.

As used herein, “dicot” refers to a dicotyledonous plant, meaning that it is a plant whose seed has two embryonic leaves (cotyledons).

As used herein, “homology”, “sequence similarity”, or “sequence identity” refers to nucleotide or amino acid sequences mean a degree of identity or similarity of two or more sequences and may be determined conventionally by using known software or computer programs such as the Best-Fit or Gap pairwise comparison programs (GCG Wisconsin Package, Genetics Computer Group, 575 Science Drive, Madison, Wis. 53711). BestFit uses the local homology algorithm of Smith and Waterman, Advances in Applied Mathematics 2:482-489 (1981), to find the best segment of identity or similarity between two sequences. Sequence comparison between two or more polynucleotides or polypeptides is generally performed by comparing portions of the two sequences over a comparison window to identify and compare local regions of sequence similarity. The comparison window is generally from about 20 to 200 contiguous nucleotides. Gap performs global alignments: all of one sequence with all of another similar sequence using the method of Needleman and Wunsch, J. Mol. Biol. 48:443-453 (1970). When using a sequence alignment program such as BestFit to determine the degree of DNA sequence homology, similarity or identity, the default setting may be used, or an appropriate scoring matrix may be selected to optimize identity, similarity or homology scores. Similarly, when using a program such as BestFit to determine sequence identity, similarity or homology between two different amino acid sequences, the default settings may be used, or an appropriate scoring matrix, such as blosum45 or blosum80, may be selected to optimize identity, similarity or homology scores.

The present invention relates to, inter alia, a recombinant nucleic acid molecule. In one embodiment, the nucleic acid molecule comprises a a nucleotide sequence that encodes a hypersensitive response substrate (HRS) protein of a plant pathogen-specific protease. The HRS protein comprises a heterologous Basidiomycete-specific protease cleavage site, wherein cleavage of the cleavage site confers to a plant an improved resistance to the Basidiomycete plant pathogen species.

The HRS protein may be a PBS1 of SEQ ID NO: 1 or an amino acid sequence having at least 80%, 85%, 90%, 91%, 92%, 93%, 94%, 95%, 96%, 97%, 98%, 99% identity to SEQ ID NO: 1, wherein the variant of the PBS1 polypeptide retains PBS1 activity, a RIN4 of SEQ ID NO: 148, or an amino acid sequence having at least 80%, 85%, 90%, 91%, 92%, 93%, 94%, 95%, 96%, 97%, 98%, 99% identity to SEQ ID NO: 148 wherein the variant of the RIN4 polypeptide retains RIN4 activity. PBS1, for example, has been previously disclosed (see, e.g. U.S. Pat. No. 9,816,102, which is herein incorporated by reference in its entirety.). In some embodiments, the HRS protein is a homolog of PBS1 from Glycine species having at least 80, at least 85%, atleast 90% sequence identity to SEQ ID NO: 1, such as the homologs of SEQ ID NO: 150-152. In further embodiments, the HRS protein may be any of the Glycine PBS1 homologs listed at www.researchsquare.com/article/rs-548382/v1 (the contents of which are incorporated by reference herein in their entirety), including but not limited to any of the following Glycine homologs: glyma.Wm82.gnm4.ann1.Glyma.20G249600.1 (SEQ ID NO: 150), glyma.Lee.gnm1.ann1.GlymaLee.20G209700.1 (SEQ ID NO: 151), glyma.Zh13.gnm1.ann1.SoyZH13_20G232600.m1 (SEQ ID NO: 152), glyso.PI483463.gnm1.ann1.GlysoPI483463.20G209800.1, glyso.W05.gnm1.ann1.Glysoja.20G055149.1, glycy.G1267.gnm1.ann1.Gcy20g056490.1, glysy.G1300.gnm1.ann1.Gsy20g055379.1, glyst.G1974.gnm1.ann1.Gst20g055837.1, glydo.G1134.gnm1.ann1.Gtt20g056563.1, glyfa.G1718.gnm1.ann1.Gfa10g029889.1, glydo.G1134.gnm1.ann1.Gtt39g108488.1, glydo.G1134.gnm1.ann1.Gtt39g108489.1, glytoD3.G1403.gnm1.ann1.Gto18g048507.1, glytoD3.G1403.gnm1.ann1.Gto18g048508.1, glyma.Wm82.gnm4.ann1.Glyma.10G298400.1, glytoD3.G1403.gnm1.ann1.Gto19g051689.1, glysy.G1300.gnm1.ann1.Gsy10g028237.1, glydo.G1134.gnm1.ann1.Gtt27g073198.1, glydo.G1134.gnm1.ann1.Gtt27g073199.1, glyma.Lee.gnm1.ann1.GlymaLee.10G256900.1, glyso.W05.gnm1.ann1.Glysoja.10G028545.1, glyfa.G1718.gnm1.ann1.Gfa10g029932.1, glyso.PI483463.gnm1.ann1.GlysoPI483463.10G254200.1, glyma.Zh13.gnm1.ann1.SoyZH13_10G276200.m1, glycy.G1267.gnm1.ann1.Gcy10g025851.1, glydo.G1134.gnm1.ann1.Gtt10g028972.1, glydo.G1134.gnm1.ann1.Gtt10g028974.1, glyma.Wm82.gnm4.ann1.Glyma.08G360600.1, glyma.Lee.gnm1.ann1.GlymaLee.08G321900.1, glyso.PI483463.gnm1.ann1.GlysoPI483463.08G318000.1, glyma.Zh13.gnm1.ann1.SoyZH13_08G339400.m1, glyso.W05.gnm1.ann1.Glysoja.08G022682.1, glycy.G1267.gnm1.ann1.Gcy8g019398.1, glydo.G1134.gnm1.ann1.Gtt30g083024.1, glytoD3.G1403.gnm1.ann1.Gto10g026268.1, glyst.G1974.gnm1.ann1.Gst8g021434.1, glysy.G1300.gnm1.ann1.Gsy8g021268.1, glydo.G1134.gnm1.ann1.Gtt8g021843.1, glyfa.G1718.gnm1.ann1.Gfa8g022769.1, glycy.G1267.gnm1.ann1.Gcy18g050446.1, glyma.Zh13.gnm1.ann1.SoyZH13_18G268200.m1, glysy.G1300.gnm1.ann1.Gsy18g049180.1, glyma.Wm82.gnm4.ann1.Glyma.18G301000.1, glydo.G1134.gnm1.ann1.Gtt18g050188.1, and glytoD3.G1403.gnm1.ann1.Gto20g056848.1.

The sequence of the glycine homologs can also be found at https://v1.legumefederation.org/data/v2/Glycine and/or www.ebi.ac.uk/enalbrowser/view/PRJEB44023.

In specific embodiments, the heterologous Basidiomycete-specific protease cleavage site is encoded by a cleavage sequence selected from the group consisting of a sequence that encodes at least one of SEQ NO: 9, 17, 21, 24, 26, 27, 28 29, or 52, or a sequence having at least 65% identity to SEQ ID NO: 9, 17, 21, 24, 26, 27, 28 29, or 52 or a sequence encoding SEQ ID NO: 9, 17, 21, 24, 26, 27, 28, 29 or 52 and having at least 1, 2 or 3 amino acid substitutions therein. Cleavage of the cleavage site results in improved resistance to the plant pathogen species. The cleavage sequence can include a sequence that encodes an amino acid motif having six amino acids. In the six amino acid motif, the first position is arginine, histidine, tryptophan, proline, leucine, glycine, tyrosine, isoleucine, threonine, glutamine, methionine, valine, or asparagine; the second position is leucine or tyrosine; the third position is tryptophan, valine, glutamic acid, isoleucine, tyrosine, threonine, methionine, arginine, lysine, leucine, or phenylalanine; the fourth position is phenylalanine or tryptophan; the fifth position is alanine, phenylalanine, valine, leucine, tryptophan, tyrosine, or serine; and the sixth position is leucine, alanine, glutamine, methionine, tryptophan, phenylalanine, tyrosine, arginine, valine, or threonine.

The cleavage sequence encodes an amino acid sequence having a number of amino acids in the range of 5 to 35 amino acids. In specific embodiments, the cleavage sequence encodes an amino acid sequence having 5 to 35 amino acids and comprising SEQ ID NO: 9; an amino acid sequence having a number of amino acids in the range of 5 to 35 and comprising SEQ ID NO: 17; an amino acid sequence having a number of amino acids in the range of 5 to 35 and comprising SEQ ID NO: 21; an amino acid sequence having a number of amino acids in the range of 5 to 35, including SEQ ID NO: 24, an amino acid sequence having a number of amino acids in the range of 5 to 35 and comprising SEQ ID NO: 26; an amino acid sequence having a number of amino acids in the range of 5 to 35 and comprising SEQ ID NO: 27; an amino acid sequence having a number of amino acids in the range of 5 to 35 and comprising SEQ ID NO: 28; an amino acid sequence having a number of amino acids in the range of 5 to 35 and comprising SEQ ID NO: 29, an amino acid sequence having a number of amino acids in the range of 5 to 35 and comprises SEQ ID NO: 52.

In other embodiments, the cleavage sequence encodes an amino acid sequence comprising at least one of RWWFAL (SEQ ID NO: 8), HWVNFL (SEQ ID NO: 9), WAELVL (SEQ ID NO: 10), PIISLA (SEQ ID NO: 11), WFYVLQ (SEQ ID NO: 12), LTEMFM (SEQ ID NO: 13), GQYFVW (SEQ ID NO: 14), YWTTLF (SEQ ID NO: 15), IQMLWA (SEQ ID NO: 16), YARFYL (SEQ ID NO: 17), LAKLWY (SEQ ID NO: 18), YFWLVR (SEQ ID NO: 19), GFWLSF (SEQ ID NO:20), TLEEWF (SEQ ID NO:21), QQLFVV (SEP ID NO: 22), MRFYFT (SEP ID NO: 23), QVFEFL (SEQ ID NO:24), VYYFYR (SEQ ID NO:25), QMIFLR (SEQ ID NO:26), IFLWSA (SEQ ID NO: 27), NLEFLY (SEQ ID NO:28), TFTFFQ (SEQ ID NO:29), or TLEEWFQVFEFL (SEQ ID NO: 52).

The HRS protein of the recombinant nucleic acid molecule may be at least one of a PBS1 or a RIN4. The PBS1 or RIN4 may be derived from Arabidopsis, Glycine, Hordeum, or Triticum, wherein the at least one PBS1 protein is an amino acid SEQ ID NO: 1, or a sequence having at least 90%, 91%, 92%, 93%, 94%, 95%, 96%, 97%, 98% or 99% identity to the nucleic acid of SEQ ID NO: 2, wherein the variant retains PBS1 activity. When the HRS protein is a RIN4, it is an amino acid SEQ ID NO: 148 or a sequence having at least 90% 91%, 92%, 93%, 94%, 95%, 96%, 97%, 98% or 99% identity to the nucleic acid of SEQ ID NO: 148, wherein the variant retains RIN4 activity.

The position of the cleavage site may vary from embodiment to embodiment. In one embodiment, the heterologous Basidiomycete-specific cleavage site or the heterologous Nematoda-specific cleavage site is located between amino acid position 150-160, 160-170, 170-180, 180-190, 190-200, 200-210, 210-220, 220-230, 230-240, 240-250, 250-260, 260-270, 270-280, 280-290, 290-300, 300-310, or 310-320 of the HRS protein. In other embodiments, the heterologous Basidiomycete-specific cleavage site or the heterologous Nematoda-specific cleavage site is located between amino acid position 150-160, 160-170, 170-180, 180-190, 190-200, 200-210, 210-220, 220-230, 230-240, 240-250, 250-260, 260-270, 270-280, 280-290, 290-300, 300-310, or 310-320 of the wild-type substrate protein, such as with reference to PBS1 (SEQ ID NO: 1). In particular embodiments, the heterologous Basidiomycete-specific cleavage site or the heterologous Nematoda-specific cleavage site is located between amino acid position 230-240, 240-250, 238-248, or 241-246 of PBS1 (SEQ ID NO: 1).

In other embodiments, the heterologous Basidiomycete-specific heterologous cleavage site is located between about amino acid position 238 to about amino acid position 248, or between about amino acid position 238 to about amino acid position 254 in reference to the modified HRS protein, such as modified PBS1 (such as any one of SEQ ID NOs: 53-97). In one particular embodiment, the heterologous Basidiomycete-specific heterologous cleavage site is located between about amino acid position 238 to about amino acid position 248 in reference to the modified PBS1 of SEQ ID NO: 74. In other embodiment, the cleavage site is located between about amino acid position 238 to about amino acid position 248 in reference to any of SEQ ID NOS: 1 and 53-73. In another particular embodiment, the cleavage site is located between about amino acid position 238 to about amino acid position 254 in reference to the modified PBS1 of SEQ ID NO: 97.

In embodiments, the modified substrate protein comprises a nucleotide sequence that, when encoded, replaces the endogenous cleavage site of the unmodified substrate protein with the heterogeneous cleavage site, such as by insertion of a 6-mer, 12-mer, 18-mer amino acid substitution of the endogenous cleavage sequence. In one particular embodiment, the endogenous cleavage sequence of PBS1 comprises a 6-mer amino acid sequence between about amino acid 241 and about amino acid 246 in reference to SEQ ID NO: 1, and the modified substrate protein is engineered to replace the endogenous 6-mer amino acid sequence between about amino acid 241 and about amino acid 246 in reference to SEQ ID NO: 1 with an alternate 6-mer amino acid sequence resulting in the modified substrate protein. In other embodiments, the modified substrate protein is engineered to replace the endogenous 6-mer amino acid sequence between about amino acid 241 and about amino acid 246 in reference to SEQ ID NO: 1 with an alternate 12-mer, 18-mer (or other multiple of 6-mer) amino acid sequence to thereby create the modified substrate protein.

In some embodiments, the nucleotide that encodes the at least one substrate protein contains a nucleotide sequence that encodes a native cleavage site. In some embodiments, the nucleotide sequence that encodes the native cleavage site is replaced by a nucleotide sequence that encodes the heterologous cleavage site, which is located between about amino acid position 238 to about amino acid position 248 in reference to any of SEQ ID NOS: 1 and 53-97, such as in reference to SEQ ID NO: 74.

In one embodiment, the Basidiomycete plant pathogen species is a Phakopsora plant pathogen species. In another embodiment, the Phakopsora plant pathogen species is Phakopsora pachyrhizi, and optionally, the disease is Asian soybean rust. Another embodiment relates to a modified substrate protein, encoded by the nucleic acid molecule previously mentioned, of a plant pathogen-specific protease expressed by a Phakopsora plant pathogen species. Said modified substrate protein comprises an amino acid sequence having a Phakopsora-specific heterologous cleavage site. The substrate protein is encoded by the recombinant nucleic acid molecule mentioned above.

In another embodiment encompasses a vector.

In embodiments, the endogenous cleavage sequence of the endogenous substrate protein is substituted with the heterogenous cleavage sequence of any of SEQ ID NOs: 8-51 and 98-145. In one example embodiment, the 6-mer endogenous cleavage sequence (SEQ ID NO: 7) of the substrate protein PBS1 (SEQ ID NO: 1) is substituted with the heterogenous cleavage sequence of any of SEQ ID NOs: 8-51 and 98-145. In other embodiments, the 6-mer endogenous cleavage sequence (SEQ ID NO: 7) of the substrate protein PBS1 (SEQ ID NO: 1) is substituted with the 12-mer heterogenous cleavage sequence of any of SEQ ID NOs: 52, and 98-145.

In embodiments, the endogenous cleavage sequence of the endogenous substrate protein is substituted with a heterogenous cleavage sequence comprising a combination or concatenation of one or more heterogenous sequences. For example, the heterogenous cleavage sequence may comprise a concatenation of two, three, four or more Basidiomycete-specific cleavage sequences, a concatenation of two, three, four or more Nematode-specific cleavage sequences, or a concatenation of two, three, four or more Basidiomycete-specific and Nematode-specific cleavage sequences. In embodiments, the modified PBS1 substrate protein comprises a heterogenous cleavage sequence comprising a first Basidiomycete-specific cleavage sequence selected from any of SEQ ID NOS: 8-29 and a second Basidiomycete-specific cleavage sequence selected from any of SEQ ID NOS: 8-29. In one particular embodiment, the heterogenous cleavage sequence PBS-R26 (SEQ ID NO: 52) is a peptide comprising an amino sequence comprising a concatenation of two different Basidiomycete-specific cleavage sequences R14 (SEQ ID NO: 14) and R17 (SEQ ID NO: 24). In other embodiments, the modified PBS1 substrate protein comprises a heterogenous cleavage sequence comprising a first Nematode-specific cleavage sequence selected from any of SEQ ID NOS: 30-51 and a second Basidiomycete-specific cleavage sequence selected from any of SEQ ID NOS: 30-51. In still other embodiments, the modified PBS1 substrate protein comprises a heterogenous cleavage sequence comprising a first Basidiomycete-specific cleavage sequence selected from any of SEQ ID NOS: 8-29 and a second Nematode-specific cleavage sequence selected from any of SEQ ID NOS: 30-51, thereby conferring the plant with improved resistance to both a Basidiomycete plant pathogen and a Nematode plant pathogen.

In another example, a substrate protein could be constructed containing both a Basidiomycete-specific and a nematode-specific cleavage site. The cleavage site can be 12-35 amino acids long. Cleavage of the cleavage site should result in increased resistance to both soy rust and nematode damage. Example sequence peptides comprising Basidiomycete-specific and Nematode-specific cleavage sites are shown at SEQ ID NOs: 98-145. These may include, for example, peptides comprising a combination of at least one Basidiomycete-specific cleavage site sequence (such as a soy rust cleavage site sequence), such as a cleavage sequence selected from SEQ ID NOs: 8-29; and at least one Nematode-specific cleavage site sequence (such as a soybean cyst nematode cleavage site sequence), such as a cleavage sequence selected from SEQ ID NOs: 98-145. In some embodiments, the peptide is a concatenated peptide comprising a concatenation of at least one soy rust cleavage site sequence and at least one soybean cyst nematode cleavage site sequence.

Thus, in other embodiments, the disclosure is directed to substrate proteins containing both a Basidiomycete-specific and a Nematode-specific cleavage site. The cleavage site can be 12-35 amino acids long. Cleavage of the cleavage site results in increased resistance to both soy rust and nematode damage.

Further provided are plants, plant cells, plant parts and seed comprising nucleic acid sequences encoding the recombinant HRS proteins disclosed herein. Plants having the recombinant polynucleotides can be propagated to produce progeny plants, and the progeny plants that have stably incorporated into its genome a polynucleotide encoding the recombinant HRS polypeptide. The term “progeny,” refers to the descendant(s) of a particular cross. The term “stable incorporation” refers to the introduction of a nucleic acid sequence into the genome of a plant and said nucleic acid sequence is capable of being inherited by the progeny thereof.

As used herein, the term “plant part” indicates a part of a plant, including single cells and cell tissues such as plant cells that are intact in plants, cell clumps and tissue cultures from which plants can be regenerated. Examples of plant parts include, but are not limited to, single cells and tissues from pollen, ovules, zygotes, leaves, embryos, roots, root tips, anthers, flowers, flower parts, fruits, stems, shoots, cuttings, and seeds; as well as pollen, ovules, egg cells, zygotes, leaves, embryos, roots, root tips, anthers, flowers, flower parts, fruits, stems, shoots, cuttings, scions, rootstocks, seeds, protoplasts, calli, and the like.

In specific embodiments, the transformed plant cell and transformed plant is a dicot and furthermore is optionally a member of the genus Glycine. Other embodiments include transgenic seed of the transformed plant. Further, in yet another embodiment, cleavage of the cleavage site from above activates a RPS5 protein and the RPS5 protein is at least 80%, 85%, 90%, 91%, 92%, 93%, 94%, 95%, 96%, 97%, 98%, 99% identical to SEQ ID NO: 3 and triggers a cell death response. In specific embodiments, a heterologous copy of the RPS5 protein or an the RPS5 protein is at least 80%, 85%, 90%, 91%, 92%, 93%, 94%, 95%, 96%, 97%, 98%, 99% identical to SEQ ID NO: 3 and triggers a cell death response is introduced into the plant or plant cell.

A nucleic acid sequence may be introduced to a plant cell by various ways, for example, by transformation, by genome modification techniques (such as by genome editing), or by breeding. In one aspect, the plant can be produced by transforming the nucleic acid sequence encoding the recombinant HRS polypeptide disclosed herein into a recipient plant. In one aspect, the method can comprise editing the genome of the recipient plant so that the resulting plant comprises a polynucleotide encoding a polypeptide disclosed above.

In some embodiments, the method comprises transforming a polynucleotide disclosed herein or an active variant or fragment thereof into a recipient plant to obtain a transgenic plant, and said transgenic plant has increased resistance to a pathogen of interest. Expression cassettes comprising polynucleotides encoding the polypeptides as described above can be used to transform plants of interest.

As used herein, the term “transgenic” and grammatical variations thereof refer to a plant, including any part derived from the plant, such as a cell, tissue or organ, in which a heterologous nucleic acid is integrated into the genome. In specific embodiments, the heterologous nucleic acid is a recombinant construct, vector or expression cassette comprising one or more nucleic acids. In other embodiments, a transgenic plant is produced by a genetic engineering method, such as Agrobacterium transformation. Through gene technology, the heterologous nucleic acid is stably integrated into chromosomes, so that the next generation can also be transgenic.

Transformation results in a transformed plant, including whole plants, as well as plant organs (e.g., leaves, stems, roots, etc.), seeds, plant cells, propagules, embryos and progeny of the same. Plant cells can be differentiated or undifferentiated (e.g., callus, suspension culture cells, protoplasts, leaf cells, root cells, phloem cells, pollen). Transformation may result in stable or transient incorporation of the nucleic acid into the cell. “Stable transformation” is intended to mean that the nucleotide construct introduced into a host cell integrates into the genome of the host cell and is capable of being inherited by the progeny thereof.

Methods for transformation typically involve introducing a nucleotide construct into a plant. In some embodiments, the transformation method is an Agrobacterium-mediated transformation. In some embodiments, the transformation method is a biolistic-mediated transformation. Transformation may also be performed by infection, transfection, microinjection, electroporation, microprojection, biolistics or particle bombardment, electroporation, silica/carbon fibers, ultrasound mediated, PEG mediated, calcium phosphate co-precipitation, poly cation DMSO technique, DEAE dextran procedure, Agrobacterium and viral mediated (e.g., Caulimoriviruses, Geminiviruses, RNA plant viruses), liposome mediated and the like.

Transformation protocols as well as protocols for introducing polypeptides or polynucleotide sequences into plants may vary depending on the type of plant or plant cell, i.e., monocot or dicot, targeted for transformation. Methods for transformation are known in the art and include those set forth in U.S. Pat. Nos. 8,575,425; 7,692,068; 8,802,934; and 7,541,517; each of which is herein incorporated by reference. See, also, Rakoczy-Trojanowska, M. (2002) Cell Mol Biol Lett. 7:849-858; Jones et al. (2005) Plant Methods, Vol. 1, Article 5; Rivera et al. (2012) Physics of Life Reviews 9:308-345; Bartlett et al. (2008) Plant Methods 4: 1-12; Bates, G. W. (1999) Methods in Molecular Biology 111:359-366; Binns and Thomashow (1988) Annual Reviews in Microbiology 42:57 Sup′/Sup5-606; Christou, P. (1992) The Plant Journal 2:275-281; Christou, P. (1995) Euphytica 85: 13-27; Tzfira et al. (2004) TRENDS in Genetics 20:375-383; Yao et al. (2006) Journal of Experimental Botany 57:3737-3746; Zupan and Zambryski (1995) Plant Physiology 107: 1041-1047.

The cells that have been transformed may be grown into plants in accordance with conventional ways. See, for example, McCormick et al. (1986) Plant Cell Reports 5:81-84. These plants may then be grown, and either pollinated with the same transformed strain or different strains, and the resulting hybrid having constitutive expression of the desired phenotypic characteristic identified. Two or more generations may be grown to ensure that expression of the desired phenotypic characteristic is stably maintained and inherited and then seeds harvested to ensure expression of the desired phenotypic characteristic has been achieved. In this manner, the present invention provides transformed seed (also referred to as “transgenic seed”) having a nucleotide construct of the invention, for example, an expression cassette of the invention, stably incorporated into their genome.

In some embodiments, the polynucleotide sequences encoding the recombinant HRS polypeptide disclosed herein can be generated through genome modification techniques which engineer the heterologous protease specific cleavage site (i.e., the heterologous Basidiomycete-specific protease cleavage site or the heterologous Nematoda-specific protease site) into the endogenous HRS sequence found in the plant's genome. Such methods include, but are not limited to, meganucleases designed against the plant genomic sequence of interest CRISPR-Cas9, TALENs, and other technologies for precise editing of genomes (Feng, et al. Cell Research 23: 1229-1232, 2013, WO 2013/026740); Cre-lox site-specific recombination; FLP-FRT recombination (Li et al. (2009) Plant Physiol 151:1087-1095); Bxbl-mediated integration (Yau et al. Plant J (2011) 701: 147-166); zinc-finger mediated integration (Wright et al. (2005) Plant J 44:693-705); Cai et al. (2009) Plant Mol Biol 69:699-709); and homologous recombination (Lieberman-Lazarovich and Levy (2011) Methods Mol Biol: 51-65).

In some embodiments, provided herein are plants transformed with and expressing gene-editing machinery as described above, which, when crossed with a target plant, result in gene editing in the target plant.

In general, gene editing may involve transient, inducible, or constitutive expression of the gene editing components or systems. Gene editing may involve genomic integration or episomal presence of the gene editing components or systems.

Gene editing generally refers to the use of a site-directed nuclease (including but not limited to CRISPR/Cas, zinc fingers, meganucleases, and the like) to cut a nucleotide sequence at a desired location. This may be to cause an insertion/deletion (“indel”) mutation, (i.e., “SDN1”), a base edit (i.e., “SDN2”), or allele insertion or replacement (i.e., “SDN3”). SDN2 or SDN3 gene editing may comprise the provision of one or more recombination templates (e.g., in a vector) comprising a gene sequence of interest that can be used for homology directed repair (HDR) within the plant (i.e., to be introduced into the plant genome). In some embodiments, the gene or allele of interest is one that is able to confer to the plant an improved trait, e.g., increased protein content and/or increased oil content. The recombination template can be introduced into the plant to be edited either through transformation or through breeding with a donor plant comprising the recombination template. Breaks in the plant genome may be introduced within, upstream, and/or downstream of a target sequence. In some embodiments, a double strand DNA break is made within or near the target sequence locus. In some embodiments, breaks are made upstream and downstream of the target sequence locus, which may lead to its excision from the genome. In some embodiments, one or more single strand DNA breaks (nicks) are made within, upstream, and/or downstream of the target sequence (e.g., using a nickase Cas9 variant). Any of these DNA breaks, as well as those introduced via other methods known to one of skill in the art, may induce HDR. Through HDR, the target sequence is replaced by the sequence of the provided recombination template comprising a polynucleotide of interest. By designing the system such that one or more single strand or double strand breaks are introduced within, upstream, and/or downstream of the corresponding region in the genome of a plant not comprising the gene sequence of interest, this region can be replaced with the template.

In some embodiments, mutations in the genes of interest described herein may be generated without the use of a recombination template via targeted introduction of DNA double strand breaks. Such breaks may be repaired through the process of non-homologous end joining (NHEJ), which can result in the generation of small insertions or deletions (indels) at the repair site. Such indels may lead to frameshift mutations causing premature stop codons or other types of loss-of-function mutations in the targeted genes.

In some embodiments, gene editing may involve transient, inducible, or constitutive expression of the gene editing components or systems in the target plant. Gene editing may also involve genomic integration or episomal presence of the gene editing components or systems in the target plant.

In certain embodiments, the nucleic acid modification or mutation is effected by a (modified) zinc-finger nuclease (ZFN) system. The ZFN system uses artificial restriction enzymes generated by fusing a zinc finger DNA-binding domain to a DNA-cleavage domain that can be engineered to target desired DNA sequences. Exemplary methods of genome editing using ZFNs can be found for example in U.S. Pat. Nos. 6,534,261; 6,607,882; 6,746,838; 6,794,136; 6,824,978; 6,866,997; 6,933,113; and 6,979,539.

In certain embodiments, the nucleic acid modification is effected by a (modified) meganuclease, which are endodeoxyribonucleases characterized by a large recognition site (double-stranded DNA sequences of 12 to 40 base pairs). Exemplary method for using meganucleases can be found in U.S. Pat. Nos. 8,163,514; 8,133,697; 8,021,867; 8,119,361; 8,119,381; 8,124,369; and 8,129,134, which are specifically incorporated by reference.

In certain embodiments, the nucleic acid modification is effected by a (modified) CRISPR/Cas complex or system. In certain embodiments, the CRISPR/Cas system or complex is a class 2 CRISPR/Cas system. In certain embodiments, said CRISPR/Cas system or complex is a type II, type V, or type VI CRISPR/Cas system or complex. The CRISPR/Cas system does not require the generation of customized proteins to target specific sequences but rather a single Cas protein can be programmed by an RNA guide (gRNA) to recognize a specific nucleic acid target, in other words the Cas enzyme protein can be recruited to a specific nucleic acid target locus (which may comprise or consist of RNA and/or DNA) of interest using said short RNA guide.

In general, the CRISPR/Cas or CRISPR system is as used herein foregoing documents refers collectively to transcripts and other elements involved in the expression of or directing the activity of CRISPR-associated (“Cas”) genes, including sequences encoding a Cas gene and one or more of, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a“spacer” in the context of an endogenous CRISPR system), or “RNA(s)” as that term is herein used (e.g., RNA(s) to guide Cas, such as Cas9, e.g. CRISPR RNA and, where applicable, transactivating (tracr) RNA or a single guide RNA (sgRNA) (chimeric RNA)) or other sequences and transcripts from a CRISPR locus. In general, a CRISPR system is characterized by elements that promote the formation of a CRISPR complex at the site of a target sequence (also referred to as a protospacer in the context of an endogenous CRISPR system). In the context of formation of a CRISPR complex, “target sequence” refers to a sequence to which a guide sequence is designed to have complementarity, where hybridization between a target sequence and a guide sequence promotes the formation of a CRISPR complex. A target sequence may comprise any polynucleotide, such as DNA or RNA polynucleotides.

In certain embodiments, the gRNA is a chimeric guide RNA or single guide RNA (sgRNA). In certain embodiments, the gRNA comprises a guide sequence and a tracr mate sequence (or direct repeat). In certain embodiments, the gRNA comprises a guide sequence, a tracr mate sequence (or direct repeat), and a tracr sequence. In certain embodiments, the CRISPR/Cas system or complex as described herein does not comprise and/or does not rely on the presence of a tracr sequence (e.g. if the Cas protein is Cas12a).

The Cas protein as referred to herein, such as but not limited to Cas9, Cas12a (formerly referred to as Cpf1), Cas12b (formerly referred to as C2c1), Cas13a (formerly referred to as C2c2), C2c3, Cas13b protein, may originate from any suitable source, and hence may include different orthologues, originating from a variety of (prokaryotic) organisms, as is well documented in the art. In certain embodiments, the Cas protein is (modified) Cas9, preferably (modified) Staphylococcus aureus Cas9 (SaCas9) or (modified) Streptococcus pyogenes Cas9 (SpCas9). In certain embodiments, the Cas protein is Cas12a, optionally from Acidaminococcus sp., such as Acidaminococcus sp. BV3L6 Cpf1 (AsCas12a) or Lachnospiraceae bacterium Cas12a, such as Lachnospiraceae bacterium MA2020 or Lachnospiraceae bacterium MD2006 (LBCas12a). See U.S. Pat. No. 10,669,540, incorporated herein by reference in its entirety. Alternatively, the Cas12a protein may be from Moraxella bovoculi AAX08_00205 [Mb2Cas12a] or Moraxella bovoculi AAX11_00205 [Mb3Cas12a]. See WO 2017/189308, incorporated herein by reference in its entirety. In certain embodiments, the Cas protein is (modified) C2c2, preferably Leptotrichia wadei C2c2 (LwC2c2) or Listeria newyorkensis FSL M6-0635 C2c2 (LbFSLC2c2). In certain embodiments, the (modified) Cas protein is C2c1. In certain embodiments, the (modified) Cas protein is C2c3. In certain embodiments, the (modified) Cas protein is Cas13b. Other Cas enzymes are available to a person skilled in the art.

Gene editing methods and compositions are also disclosed in U.S. Pat. Nos. 10,519,456 and 10,285,348 82, the entire content of which is herein incorporated by reference.

The gene-editing machinery (e.g., the DNA modifying enzyme) introduced into the plants can be controlled by any promoter that can drive recombinant gene expression in plants. In some embodiments, the promoter is a constitutive promoter. In some embodiments, the promoter is a tissue-specific promoter, e.g., a pollen-specific promoter or a sperm cell specific promoter, a zygote specific promoter, or a promoter that is highly expressed in sperm, eggs and zygotes (e.g., prOsActin1). Suitable promoters are disclosed in U.S. Pat. No. 10,519,456, the entire content of which is herein incorporated by reference.

In another aspect, provided herein is a method of editing plant genomic DNA. In some embodiments, the method comprises using a first soybean plant expressing a DNA modification enzyme and at least one optional guide nucleic acid as described above to pollinate a target plant comprising genomic DNA to be edited.

Also included are methods of protecting a plant from infection by a Basidiomycete plant pathogen species or enhancing plant pathogen resistance to a Basidiomycete plant pathogen species. In one embodiment, a method comprises the steps of introducing a nucleotide sequence to the plant that encodes an HRS protein of a plant pathogen-specific protease, where the HRS protein has a heterologous Basidiomycete-specific cleavage site and cleavage of the cleavage site confers resistance or enhanced resistance to the Basidiomycete plant pathogen species. In an exemplary embodiment, the Basidiomycete plant pathogen is Phakopsora pachyrhizi and the disease is Asian Soybean Rust, the HRS protein is at least one of a PBS1 of SEQ ID NO: 1 or an amino acid sequence having at least 80%, 85%, 90%, 91%, 92%, 93%, 94%, 95%, 96%, 97%, 98%, or 99% identity to SEQ ID NO: 1, wherein the variant retains PBS1 activity. In another embodiment, the recombinant HSR sequence comprises RIN4 of SEQ ID NO: 148 or an amino acid sequence having at least 80%, 85%, 90%, 91%, 92%, 93%, 94%, 95%, 96%, 97%, 98%, or 99% identity to SEQ ID NO: 148, wherein the variant retains RIN4 activity. In other embodiments, the recombinant substrate protein has a heterologous Phakopsora-specific cleavage site, the heterologous cleavage site is encoded by a cleavage sequence selected from the group consisting of a sequence that encodes at least one of SEQ NO: 9, 17, 21, 24, 26, 27, 28 or 29, or a sequence having at least 90%, 91%, 92%, 93%, 94%, 95%, 96%, 97%, 98%, or 99% identity to SEQ ID NO: 9, 17, 21, 24, 26, 27, 28 or 29, and cleavage of the cleavage site confers improved resistance to Phakopsora pachyrhizi and Asian Soybean Rust.

In other embodiments, a recombinant nucleic acid molecule comprises a nucleotide sequence encoding at least one hypersensitive response substrate (HRS) protein of a plant pathogen specific protease. The plant pathogen specific protease is expressed by a nematode species and the HRS protein comprises a heterologous a nematode-specific protease cleavage sites, wherein cleavage of said cleavage site confers resistance to the nematode species. In specific embodiments the recombinant nucleic acid molecule is operably linked to a promoter active in a plant. The HRS protein is at least one of a PBS1 of SEQ ID NO: 1, an amino acid sequence having at least 80%, 85%, 90%, 91%, 92%, 93%, 94%, 95%, 96%, 97%, 98%, or 99% identity to SEQ ID NO: 1, wherein the variant retains PBS1 activity; a RIN4 of SEQ ID NO: 148, or an amino acid sequence having at least 80%, 85%, 90%, 91%, 92%, 93%, 94%, 95%, 96%, 97%, 98%, or 99% identity to SEQ ID NO: 148, wherein the variant retains RIN4 activity. The heterologous nematode-specific protease cleavage site from above is encoded by a cleavage sequence selected from the group consisting of a sequence that encodes at least one of SEQ ID NO: 34, 35, 48, 49, 50, or 51, or a sequence having at least 65% identity to SEQ ID NO: 34, 35, 48, 49, 50, or 51 or a sequence having at least 1, 2 or 3 amino acid substitutions in SEQ ID NO: 34, 35, 48, 49, 50, or 51. Cleavage of the cleavage site results in improved or enhanced resistance to the Nematoda species. In one embodiment, the cleavage sequence includes a sequence that encodes an amino acid motif having six amino acids. In the amino acid motif, the first position is threonine, leucine, valine, histidine, lysine, serine, tyrosine, phenylalanine, methionine, glutamine, arginine, or tryptophan; the second position is alanine, isoleucine, methionine, lysine, leucine, threonine, proline, tyrosine, asparagine, glutamine, phenylalanine, glutamic acid, or histidine; the third position is leucine; the fourth position is methionine; the fifth position is isoleucine, lysine, histidine, leucine, tyrosine, methionine, glycine, proline, threonine, valine, glutamic acid, glutamine, or arginine; and the sixth position is histidine, asparagine, alanine, methionine, proline, isoleucine, phenylalanine, leucine, aspartic acid, glutamine, lysine, or arginine.

In another embodiment, the cleavage site sequence of the recombinant nucleic acid molecule encodes at least one of an amino acid sequence having a number of amino acids in the range of 5 to 35. In specific embodiments, the cleavage site sequence of the recombinant nucleic acid molecule encodes at least one of an amino acid sequence having a number of amino acids in the range of 5 to 3 and comprises SEQ ID NO: 34 therein; an amino acid sequence having a number of amino acids in the range of 5 to 35 and comprises SEQ ID NO: 35 therein; an amino acid sequence having a number of amino acids in the range of 5 to 35 and comprises SEQ ID NO: 48 therein; an amino acid sequence having a number of amino acids in the range of 5 to 35 and comprises SEQ ID NO: 49 therein; an amino acid sequence having a number of amino acids in the range of 5 to 35 and comprises SEQ ID NO: 50 therein; and an amino acid sequence having a number of amino acids in the range of 5 to 35 and comprises SEQ ID NO: 51.

In another embodiment, the cleavage site sequence encodes at least one of TAMRIH (SEQ ID NO: 30), LIMQKN (SEQ ID NO: 31), VMLMHA (SEQ ID NO: 32), VKSFLM (SEQ ID NO: 33), HLYLYH (SEQ ID NO: 34), LMFKMP (SEQ ID NO: 35), KLMHGI (SEQ ID NO: 36), LTPLMI (SEQ ID NO: 37), SPLMKH (SEQ ID NO: 38), YYIAPL (SEQ ID NO: 39), KNMMTF (SEQ ID NO: 40), FYTKHL (SEQ ID NO: 41), MQSAID (SEQ ID NO: 42), QFEGVL (SEQ ID NO: 43), MQHVIA (SEQ ID NO: 44), HNRIEQ (SEQ ID NO: 45), RIWKQM (SEQ ID NO: 46), KLRQRH (SEQ ID NO: 47), WEGLMK (SEQ ID NO: 48), FELMKA (SEQ ID NO: 49), LMKLHN (SEQ ID NO: 50), or KHGLMR (SEQ ID NO: 51).

The at least one HRS protein of the recombinant nucleic acid molecule is at least one of a PBS1 or RIN4. The HRS protein can comprise a PBS1 protein and is SEQ ID NO: 1, or a sequence having at least 90%, 91%, 92%, 93%, 94%, 95%, 96%, 97%, 98%, or 99% identity to a nucleic acid of SEQ ID NO: 2, wherein the variant retains PBS1 activity. The HRS protein can comprise a RIN4 protein and is SEQ ID NO: 148, or a sequence having at least 90%, 91%, 92%, 93%, 94%, 95%, 96%, 97%, 98%, or 99% identity to a nucleic acid of SEQ ID NO: 149, wherein the variant retains RIN4 activity. In some embodiments, the HRS protein is derived from Arabidopsis, Glycine, Hordeum, or Triticum.

The position of the cleavage site may vary from embodiment to embodiment. In one embodiment, the heterologous Nematode-specific cleavage site is located between amino acid position 150-160, 160-170, 170-180, 180-190, 190-200, 200-210, 210-220, 220-230, 230-240, 240-250, 250-260, 260-270, 270-280, 280-290, 290-300, 300-310, or 310-320. In other embodiments, the heterologous nematode-specific protease cleavage site of the recombinant nucleic acid molecule is positioned between about amino acid position 238 to about amino acid position 248 in reference to SEQ ID NO: 79. In other embodiments, the cleavage site is located between about amino acid position 238 to about amino acid position 248 in reference to SEQ ID NOS: 75-78 and SEQ ID NOS: 80-96. In some embodiments of the recombinant nucleic acid molecule, the nucleotide sequence encoding the at least one HRS protein contains a sequence encoding a native cleavage site, e.g. in addition to the heterologous cleavage sites described herein. In some embodiments, the sequence encoding the native cleavage site is replaced by the nucleotide sequence that encodes the heterologous cleavage site.

In one embodiment, the nematode species of the recombinant nucleic acid molecule is selected from the group consisting of Heterodera, Globodera, Meloidogyne, or Rotylenchulus. In another embodiment, the nematode plant species is Heterodera glycines. In yet another embodiment, there is a modified HRS protein of a plant pathogen specific protease expressed by a nematode species. The modified HRS protein comprises an amino acid sequence having a nematode specific heterologous cleavage site and the modified HRS protein is encoded by the recombinant nucleic acid molecule.

Also included are vectors comprising the recombinant nucleic acid molecules disclosed herein as well as a transformed plant cells and transformed plants comprising the recombinant nucleic acid molecule. In many embodiments, the transformed plant cell and transformed plant are dicot, e.g., a member of the Glycine genus. Embodiments also include transgenic seed of a transformed plant. In an embodiment, cleavage of the cleavage site activates a RPS5 resistance protein. The RPS5 protein is at least 80%, 85%, 90%, 91%, 92%, 93%, 94%, 95%, 96%, 97%, 98%, or 99% identical to SEQ ID NO: 3 and triggers a cell death response.

In another embodiment, there is a method of protecting a plant from infection by a nematode plant pathogen species or enhancing a plants resistance from infection by a nematode plant pathogen species. The method comprises introducing a nucleotide sequence to the plant that encodes the recombinant HRS protein of a plant pathogen-specific protease, where the recombinant HRS protein comprises a heterologous nematode-specific protease cleavage site and the cleavage of the cleavage site confers resistance to the Nematoda plant pathogen species. In specific embodiments, the nematode plant pathogen species is Heterodera glycines, and the at least one substrate protein has a Heterodera-specific heterologous cleavage site. The heterologous cleavage site is encoded by a cleavage sequence selected from the group consisting of a sequence that encodes at least one of SEQ NO: 34, 35, 48, 49, 50, or 51, or a sequence having at least 65% identity to SEQ ID NO: 34, 35, 48, 49, 50, or 51. Cleavage of the cleavage site confers improved resistance to Heterodera glycines.

In a separate embodiment, it is a recombinant nucleic acid molecule comprising a promoter operably linked to a nucleotide sequence that encodes at least one HRS protein of a plant pathogen-specific protease expressed by either of a Phakopsora and Heterodera plant pathogen species. The at least one HRS protein has a Phakopsora-Heterodera specific heterologous cleavage site, and cleavage of the cleavage site confers improved resistance to at least one of Phakopsora and Heterodera plant pathogen species. In this recombinant nucleic acid molecule, the at least one HRS protein is at least one of a PBS1 of SEQ ID NO: 1 or an amino acid sequence having at least 80% identity to SEQ ID NO: 1, or the at least one HRS protein is at least one of a RIN4 of SEQ ID NO: 148 or an amino sequence having at least 80% identity to SEQ ID NO: 148. The heterologous cleavage site of the recombinant nucleic acid is encoded by a cleavage sequence selected from the group consisting of a sequence that encodes at least one of SEQ NO: 98-145, or a sequence having at least 65% identity to SEQ ID NO: 98-145. Cleavage of the cleavage site results in improved resistance to the plant pathogen species. In another embodiment, it is an isolated cleavage site for insertion into an HRS protein to create engineered resistance. The cleavage site peptide is selected from the group consisting of SEQ ID NOs: 8-52, 98-147.

Non-limiting embodiments include a recombinant nucleic acid molecule comprising: a nucleotide sequence that encodes a hypersensitive response substrate (HRS) protein of a plant pathogen-specific protease, wherein the HRS protein comprises a heterologous Basidiomycete-specific protease cleavage site. In some embodiments of the recombinant nucleic acid molecule, the HRS protein comprises: a) PBS1 polypeptide; b) a PBS1 polypeptide as set forth in SEQ ID NO: 1; c) an amino acid sequence having at least 80%, 85%, 90%, 95%, 96%, 97%, 98%, or 99% sequence identity to SEQ ID NO: 1, wherein said polypeptide retains PBS1 activity; d) a RIN4 polypeptide; e) a RIN4 polypeptide as set forth in SEQ ID NO: 148; or f) an amino acid sequence having at least 80% 85%, 95%, 96%, 97%, 98%, or 99% sequence identity to SEQ ID NO: 148, wherein said polypeptide retains RIN4 activity. In embodiments of the recombinant nucleic acid molecule, the heterologous Basidiomycete-specific cleavage site comprises a cleavage sequence selected from the group consisting of SEQ ID NO: 9, 17, 21, 24, 26, 27, 28 or 29, or a sequence having at least 65% identity to SEQ ID NO: 9, 17, 21, 24, 26, 27, 28 or 29. In embodiments, the heterologous Basidiomycete-specific cleavage site comprises a cleavage sequence of SEQ ID NO: 52 or a sequence having at least 65% identity to SEQ ID NO: 52. In embodiments of the recombinant nucleic acid molecule, the heterologous Basidiomycete-specific cleavage site encodes at least one of: a) an amino acid sequence having a number of amino acids in the range of 5 to 35 and comprising SEQ ID NO: 9 therein; b) an amino acid sequence a having number of amino acids in the range of 5 to 35, and comprising SEQ ID NO: 17 therein; c) an amino acid sequence having a number of amino acids in the range of 5 to 35, and comprising SEQ ID NO: 21 therein; d) an amino acid sequence having a number of amino acids in the range of 5 to 35, and comprising SEQ ID NO: 24 therein; e) an amino acid sequence having a number of amino acids in the range of 5 to 35, and comprising SEQ ID NO: 26 therein; f) an amino acid sequence having a number of amino acids in the range of 5 to 35, and comprising SEQ ID NO: 27 therein; g) an amino acid sequence having a number of amino acids in the range of 5 to 35, and comprising SEQ ID NO: 28 therein; h) an amino acid sequence having a number of amino acids in the range of 5 to 35, and comprising SEQ ID NO: 29 therein; or i) an amino acid sequence having a number of amino acids in the range of 5 to 35, and comprising SEQ ID NO: 52 therein. In particular embodiments, the cleavage sequence of the recombinant nucleic acid molecule comprises a nucleotide sequence encoding at least one of HWVNFL (SEQ ID NO: 9), YARFYL (SEQ ID NO: 17), TLEEWF (SEQ ID NO: 21), QVFEFL (SEP ID NO: 24), QMIFLR (SEQ ID NO: 26), IFLWSA (SEQ ID NO: 27), NLEFLY (SEQ ID NO: 28), or TFTFFQ (SEQ ID NO: 29). In other embodiments, the cleavage sequence comprises a nucleotide sequence encoding TLEEWFQVFEFL (SEQ ID NO: 52).

In non-limiting embodiments, the HRS protein is derived from Arabidopsis, Glycine, Hordeum, or Triticum. In embodiments of the recombinant nucleic acid molecule, the Basidiomycete-specific heterologous cleavage site is located between about amino acid position 238 to about amino acid position 248 in reference to any of SEQ ID NO: 1 and SEQ ID NO: 53-97. In embodiments, the heterologous Basidiomycete-specific protease is from a Phakopsora plant pathogen species. In particular embodiments, the Phakopsora plant pathogen species is Phakopsora pachyrhizi and the disease is Asian Soybean Rust. In embodiments of the recombinant nucleic acid molecule, cleavage of the heterologous Basidiomycete-specific protease cleavage site activates a RPS5 resistance protein. In particular embodiments, the RPS5 protein is at least 80% identical to SEQ ID NO: 3 and triggers a hypersensitive cell death response. In embodiments of the recombinant nucleic acid molecule, an endogenous plant pathogen-specific protease cleavage site of the HRS protein is replaced by the heterologous Basidiomycete-specific protease cleavage site. In embodiments of the recombinant nucleic acid molecule, the nucleotide sequence that encodes the hypersensitive response substrate (HRS) protein is operably linked to a promoter active in a plant. In embodiments, the recombinant nucleic acid molecule further comprises an expression cassette comprising a promoter active in a plant operably linked to an R-gene that is activated by the HRS protein following cleavage at the Basidiomycete-specific protease cleavage site.

In non-limiting embodiments, a vector is provided comprising the recombinant nucleic acid molecule in accordance with any of the above embodiments. In other non-limiting embodiments, a recombinant protein is provided comprising a modified hypersensitive response substrate (HRS) protein of a plant pathogen-specific protease, wherein the modified HRS protein comprises a heterologous Basidiomycete-specific protease cleavage site, and wherein the modified HRS protein is encoded by the recombinant nucleic acid molecule according to any of the above-described embodiments.

In further embodiments, a plant, plant cell, plant part or a seed is provided comprising the recombinant nucleic acid molecule according to any of the above-described embodiments. In particular embodiments of the plant, plant cell, plant part or a seed, the plant, plant part or plant cell is a dicot, such as a member of the genus Glycine.

Non-limiting embodiments include a method of enhancing plant pathogen resistance in a plant from infection by a Basidiomycete plant pathogen species, the method comprising: expressing in the plant a nucleotide sequence that encodes a hypersensitive response substrate (HRS) protein of a plant pathogen-specific protease comprising a heterologous Basidiomycete-specific cleavage site as described in any one of above embodiments, wherein cleavage of the heterologous Basidiomycete-specific cleavage site by the protease confers resistance to a disease caused by the Basidiomycete plant pathogen species. In embodiments, said nucleotide sequence that encodes the HRS protein is introduced into the plant by transformation. In embodiments, said nucleotide sequence that encodes the HRS protein is introduced into the plant by genome modification. In embodiments of the method, the Basidiomycete plant pathogen species is Phakopsora pachyrhizi and the disease is Asian Soybean Rust (ASR); wherein the heterologous Basidiomycete-specific cleavage site is a Phakopsora-specific heterologous cleavage site comprising a cleavage sequence selected from the group consisting of SEQ NO: 9, 17, 21, 24, 26, 27, 28 or 29 or a sequence having at least 65% identity to SEQ ID NO: 9, 17, 21, 24, 26, 27, 28 or 29; and wherein cleavage of the HRS protein at the cleavage site by the protease confers improved resistance to Phakopsora pachyrhizi and ASR.

Non-limiting embodiments are provided of a recombinant nucleic acid molecule comprising: a nucleotide sequence that encodes a hypersensitive response substrate (HRS) protein of a plant pathogen-specific protease, wherein the HRS protein has a heterologous Nematoda-specific protease cleavage site. In embodiments of the recombinant nucleic acid molecule, the HRS protein comprises: a PBS1 polypeptide; a PBS1 polypeptide as set forth in SEQ ID NO: 1, an amino acid sequence having at least 80%, 85%, 90%, 95%, 96%, 97%, 98%, or 99% sequence identity to SEQ ID NO: 1, wherein said polypeptide retains PBS1 activity; a RIN4 polypeptide; a RIN4 polypeptide as set forth in SEQ ID NO: 148; or an amino acid sequence having at least 80% 85%, 95%, 96%, 97%, 98%, or 99% sequence identity to SEQ ID NO: 148, wherein said polypeptide retains RIN4 activity. In particular embodiments of the recombinant nucleic acid molecule, the heterologous Nematoda-specific cleavage site comprises a cleavage sequence selected from the group consisting of SEQ ID NO: 34, 35, 48, 49, 50, or 51, or a sequence having at least 65% identity to SEQ ID NO: 34, 35, 48, 49, 50, or 51. In embodiments of the recombinant nucleic acid molecule, the cleavage sequence comprises a nucleic acid sequence encoding: an amino acid sequence having a number of amino acids in the range of 5 to 35, and comprising SEQ ID NO: 34 therein; an amino acid sequence having a number of amino acids in the range of 5 to 35, and comprising SEQ ID NO: 35 therein; an amino acid sequence having a number of amino acids in the range of 5 to 35, and comprising SEQ ID NO: 48 therein; an amino acid sequence having a number of amino acids in the range of 5 to 35, and comprising SEQ ID NO: 49 therein; an amino acid sequence having a number of amino acids in the range of 5 to 35, and comprising SEQ ID NO: 50 therein; or an amino acid sequence having a number of amino acids in the range of 5 to 35, and comprising SEQ ID NO: 51 therein. In particular embodiments of the recombinant nucleic acid molecule, the HRS protein is derived from Arabidopsis, Glycine, Hordeum, or Triticum. In embodiments, the heterologous Nematoda-specific cleavage site is located between about amino acid position 238 to about amino acid position 248 in reference to any of SEQ ID NO: 71 and SEQ ID NOs: 53-97. In embodiments of the recombinant nucleic acid molecule, an endogenous plant protease cleavage site of the nucleotide sequence encoding the HRS protein is replaced with a nucleotide sequence for the heterologous Nematoda-specific cleavage site. In particular embodiments of the recombinant nucleic acid molecule, the Nematoda plant pathogen species is Heterodera, Globodera, Meloidogyne, or Rotylenchulus, such as wherein the Nematoda plant pathogen species is Heterodera glycines.

In non-limiting embodiments of the recombinant nucleic acid molecule, the nucleotide sequence that encodes the hypersensitive response substrate (HRS) protein is operably linked to a promoter active in a plant. In further embodiments, the recombinant nucleic acid molecule further comprises an expression cassette comprising a promoter active in a plant operably linked to an R-gene encoding a resistance protein that is activated by the HRS protein following cleavage at the Nematoda-specific protease cleavage site. In embodiments of the recombinant nucleic acid molecule, cleavage of the Nematoda-specific protease cleavage site activates the resistance protein RPS5 and triggers a localized hypersensitive cell death response. In particular embodiments, the resistance protein RPS5 is SEQ ID NO: 3 or a protein having at least 80% sequence identity to SEQ ID NO: 3 and triggers a hypersensitive cell death response.

Non-limiting embodiments are provided for a vector comprising the recombinant nucleic acid molecule according to any of the embodiments disclosed above. Non-limiting embodiments are provided for a plant, plant part, plant cell or a seed, comprising the recombinant nucleic acid molecule according to any of the embodiments disclosed above. In particular embodiments of the plant, plant part, plant cell or a seed, the plant, plant part, plant cell or seed is a dicot, such as a member of the genus Glycine. In embodiments, a transgenic seed of the plant of any of the above embodiments is provided. In embodiments, a transformed plant cell of the plant of any of the above embodiments is provided.

Non-limiting embodiments include a recombinant protein comprising a modified hypersensitive response substrate (HRS) protein of a plant pathogen-specific protease, wherein the modified HRS protein comprises an amino acid sequence having a heterologous Nematoda-specific-cleavage site, and wherein the modified HRS protein is encoded by the recombinant nucleic acid molecule according to any of the above embodiments.

Non-limiting embodiments are provided for a method of enhancing plant pathogen resistance in a plant from infection by a Nematoda plant pathogen species, the method comprising: expressing in the plant a nucleotide sequence that encodes a hypersensitive response substrate (HRS) protein of a plant pathogen-specific protease comprising a heterologous Nematoda-specific cleavage site as described in any one of the above embodiments, wherein cleavage of the heterologous Nematoda-specific cleavage site by the protease confers the plant with resistance to a disease caused by the Nematoda plant pathogen species. In embodiments of the method, the Nematoda plant pathogen species is Heterodera glycines and the disease is Soybean Cyst Nematode (SCN); wherein the heterologous Nematoda-specific cleavage site is a Heterodera-specific heterologous cleavage site comprising a cleavage sequence selected from the group consisting of SEQ NO: 34, 35, 48, 49, 50, or 51 or a sequence having at least 65% identity to SEQ ID NO: 34, 35, 48, 49, 50, or 51; and wherein cleavage of the HRS protein at the cleavage site by the protease confers improved resistance to Heterodera glycines and SCN.

Non-limiting embodiments are provided for a recombinant nucleic acid molecule comprising: a promoter operably linked to a nucleotide sequence that encodes a HRS protein of a plant pathogen-specific protease, and wherein the HRS protein has a heterologous Phakopsora-Heterodera specific cleavage site, wherein cleavage of the cleavage site confers improved resistance to at least one of Phakopsora and Heterodera plant pathogen species. In embodiments of the recombinant nucleic acid molecule, the HRS protein is at least one of a PBS1 of SEQ ID NO: 1 or an amino acid sequence having at least 80%, 90%, 95%, 96%, 97%, 98%, or 99% identity to SEQ ID NO: 1, or wherein the at least one HRS protein is at least one of a RIN4 of SEQ ID NO: 148, or an amino sequence having at least 80%, 90%, 95%, 96%, 97%, 98%, or 99% identity to SEQ ID NO: 148. In embodiments of recombinant nucleic acid molecule, the heterologous cleavage site includes a first cleavage sequence for Phakopsora selected from at least one of SEQ ID NOs: 8-29, and a second cleavage sequence for Heterodera selected from at least one of SEQ ID NOs 30-51. In embodiments of the recombinant nucleic acid molecule, the heterologous cleavage site includes a cleavage sequence selected from the group consisting of a sequence that encodes at least one of SEQ NOs: 98-145, or a sequence having at least 65% identity to SEQ ID NOs: 98-145, wherein cleavage of the cleavage site results in improved resistance to at least one of Phakopsora and Heterodera.

Non-limiting embodiments are provided for an isolated cleavage site peptide for insertion into an HRS protein to create engineered resistance, wherein the cleavage site peptide is selected from the group consisting of SEQ ID NOS: 8-52, and 98-145.

### EXAMPLES

The disclosure will be more fully understood upon consideration of the following non-limiting examples, which are offered for purposes of illustration, not limitation.

### Example 1. Selecting and Testing Soy Rust Cleavage Site Specificity in Planta Using Nicotiana benthamiana and Nicotiana tabacum

Based on motif analysis of more than 10,000 peptides, we selected 22 Basidiomycete-specific cleavage site peptides for evaluation in N. benthamiana and N. tabacum, listed herein as R1-R22 (SEQ ID NOs 8-29). Each of these peptides was 6 amino acids long and was assayed for conferring improved resistance to Phakopsora. Herein the peptide sequences assayed for conferring improved resistance to Phakopsora-induced soy rust are also referred to as soy rust cleavage site sequences. For generating PBS1 variants (that is, modified PBS1 proteins having a heterogenous cleavage site sequence), the nucleotide sequence encoding the wild type peptide was replaced with one of the six amino acid long peptides constructed in a binary vector driven by plant promoter prGmUbi. The gene encoding the corresponding R protein that interacts with the substrate protein to mount a hypersensitive response, RPS5, was included in another binary construct driven by plant promoter prMt12344. All expression constructs were transformed into Agrobacterium strain EHA101 RecA for experiments in planta. An example amino acid motif for a Basidiomycete-specific, soy rust cleavage site is shown at SEQ ID NO: 146.

During evaluations with N. benthamiana, nearly all PBS1 variants, when transformed with the RPS5 protein, produced cell death or a hypersensitive response, regardless of whether the plant pathogen specific proteases were present or not. These results possibly indicate that N. benthamiana may contain a protease with similar substrate specificity as the pathogen specific proteases. Therefore, we were unable to differentiate responses of PBS1 variant cleavage by pathogen specific proteases.

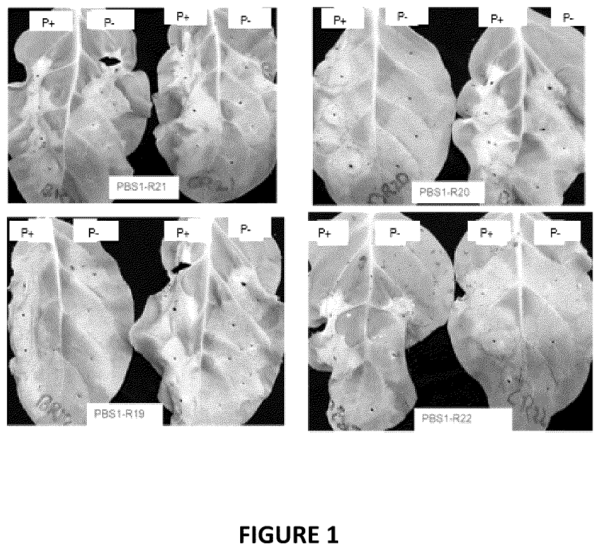

However, when we evaluated PBS1 variants using N. tabacum, there were variants where we observed a reduction (e.g., partial reduction or complete reduction) of plant protease cleavage. We also observed several PBS1 variants that showed pathogen specific protease cell death. As seen in Table 1, the cell death was rated from 0-4, with 0 representing no cell death and 4 representing the strongest cell death response. P⁺ represents the presence of the pathogen specific proteases while P⁻ represents protease absence. The peptides displayed in Table 1 exhibited specific cell death response with ratings ranging from 3-4. See FIG. 1 for leaf illustration examples of this data.

### Example 2. Efficacy with Soy Rust Cleavage Sites of Variable Length

Embodiments of the invention also include cleavage sites of different length, e.g., 5 to 15 amino acids. To illustrate the effect of cleavage site length, we created PBS1 variants with two concatenated 6-mer peptides. In one example, we concatenated PBS1-R14 and PBS1-R17 (coded as PBS1-R26). PBS1-R26 contains the 6 amino acids from both R14 (SEQ ID NO: 14) and R17 (SEQ ID NO: 24) resulting in TLEEWFQVFEL (SEQ ID NO: 52). The concatenated variant led to cleavage site dependent cell death in N. tabacum. These results, a cell death rating of 4, can also be seen in Table 1. The construct comprising the concatenated PBS1 was then stacked with RPS5 in a molecular stack for soy transformation. In other examples, cleavage sites may be lengthened or shortened as desired so long as the modification confers improved resistance to the target pathogen such as to Phakopsora plant pathogen species. In other examples, cleavage sites may be as many as five amino acids, six amino acids, seven amino acids, eight amino acids, nine amino acids, ten amino acids, eleven amino acids, twelve amino acids, thirteen amino acids, fourteen amino acids or fifteen amino acids so long as the modification confers improved resistance to the target pathogen, such as to Phakopsora plant pathogen species.

TABLE 1
Cleavage sites exhibiting the
greatest cell death response

| 6-mer sequence | Code | P+ | P− |
|---|---|---|---|
| HWVNFL (SEQ ID NO: 9) | PBS1-R2 | 3 | 0 |
| YARFYL (SEQ ID NO: 17) | PBS1-R10 | 4 | 0 |
| TLEEWF (SEQ ID NO: 21) | PBS1-R14 | 4 | 0 |
| QVFEFL (SEQ ID NO: 24) | PBS1-R17 | 4 | 0 |
| QMIFLR (SEQ ID NO: 26) | PBS1-R19 | 4 | 0 |
| IFLWSA (SEQ ID NO: 27) | PBS1-R20 | 4 | 0 |
| NLEFLY (SEQ ID NO: 28) | PBS1-R21 | 4 | 0 |
| TFTFFQ (SEQ ID NO: 29) | PBS1-R22 | 4 | 0 |
| TLEEWFQVFEFL | PBS1-R26 | 4 | 0 |
| (SEQ ID NO: 52) | | | |

### Example 3. PBS1 and RPS5 Co-Transformation in Soybean

The constructs comprising the PBS1 variants that exhibited plant pathogen specific protease dependent cell death (as listed in Table 1) were submitted for soybean transformation with RPS5 to investigate whether the PBS1 variants would trigger soy rust resistance. We co-transformed PBS1 variants with RPS5 to evaluate the following: 1) if the soybean transformation line contained functional RPS5 and 2) if so, to determine if the PBS1 variants and RPS5 would function together as the PBS1 variants are of Arabidopsis origin. From the co-transformations, we generated 3 GM soybean materials: 1) with only the PBS1 variant, 2) with only the RPS5 protein, and 3) with both the PBS1 variant as well as the RPS5 protein. With these materials, we evaluated rust specific resistance.

### Example 4. Rust Resistance in Soybean

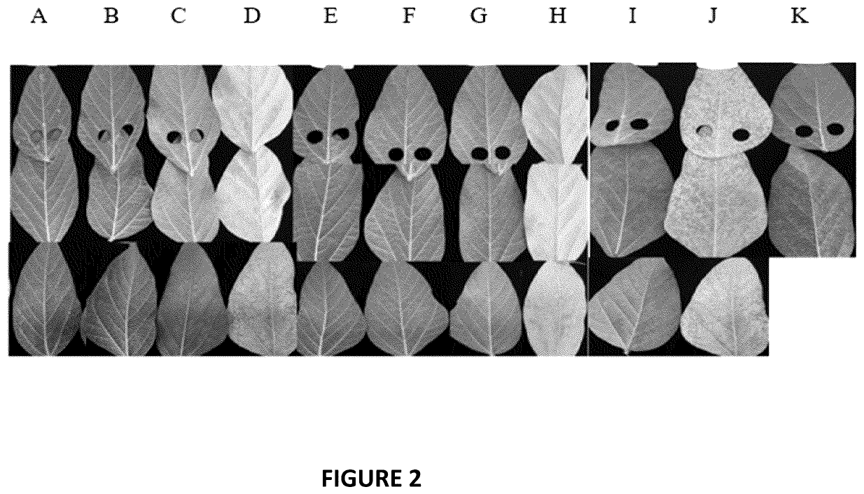

We evaluated each of the materials produced from transformation on 2-3 different rust strains, referred to herein as rust1, rust2, and rust3. Control events generated with only RPS5 (non-efficacious event G40; construct 25331) did not show observable resistance on any of the three strains. See FIG. 2, top row, middle row, and bottom row.

We observed 3 PBS1 variants, PBS1-R17, PBS1-R19, and PBS1-R20, that showed strong (more than 85% efficacy) resistance to the three soy rust strains compared to control event G40. A slight increase in resistance occurred when RPS5 was also present based on fungal mass quantifications. These results indicate that soybean's RPS5 homolog was able to interact with the PBS1 variant in triggering the hypersensitive response in the plant. Further, this indicates that a greater amount of RPS5 in the plant (such as produced by overexpression of RPS5 in the plant) can result in increased levels of resistance through interaction between the RPS5 and the PBS1 variant. Phenotypic data supporting this is shown in FIG. 2.

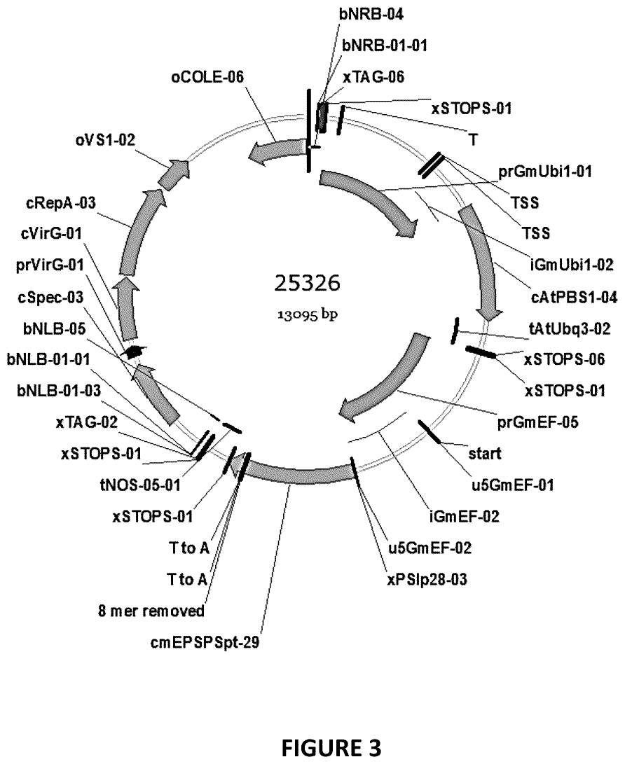

FIG. 3 represents an image of the binary vector containing the PBS1-R17 variant. This variant contains the R17 cleavage site peptide. The cleavage site is modified in the Arabidopsis thaliana PBS1 (AvrPphB susceptible 1) gene with mutations in six amino acids from the wild type (DKSHVS; SEQ ID NO: 7) to the R17 peptide (QVFEFL; SEQ ID NO: 24).

TABLE 2

| | | | | RPS5 | |
|---|---|---|---|---|---|
| Event | Construct | Copy # | 6mer code | Construct | Copy # |
| G7 | 25326 | 1 | R17 | 25331 | 1 |
| G61 | 25326 | >2 | R17 | 25331 | 1 |
| G80 | 25326 | >2 | R17 | 25331 | 0 |
| G1 | 25327 | 1 | R19 | 25331 | 1 |
| G3 | 25327 | >2 | R19 | 25331 | 0 |
| G2 | 25328 | >2 | R20 | 25331 | 2 |
| G40 | 25328 | 0 | R20 | 25331 | >2 |
| G97 | 25328 | >2 | R20 | 25331 | 2 |

### Example 4. Selecting and Testing Soybean Cyst Nematode Cleavage Site Specificity in Planta Using Nicotiana tabacum

Based on motif analysis of more than 300,000 peptides, we selected 25 Namatide-specific cleavage site peptides for evaluation in N. tabacum, listed herein as S1-S24 (SEQ ID NOs 30-51). These cleavage site peptides are all 6 amino acids long. Herein the peptide sequences assayed for conferring improved resistance to Heterodera-induced Soybean cysts are also referred to as soybean cyst nematode cleavage site sequences. For PBS1 variants, the wild type peptide was replaced with one of the six amino acid long peptides constructed in a binary vector driven by prGmUbi. The RPS5 protein was constructed in another binary construct driven by prMt12344. All constructs were transformed into Agrobacterium strain EHA101 RecA for experiments in planta. An example amino acid motif for a Nematode-specific, soybean cyst cleavage site is shown at SEQ ID NO: 146.

### Example 5. Efficacy with Soybean Cyst Nematode Cleavage Sites in N. tabacum

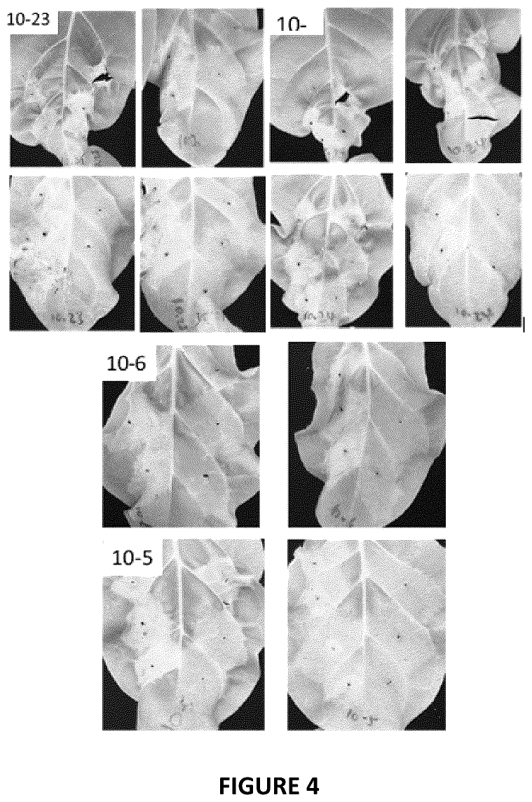

We observed several PBS1 variants that showed pathogen specific protease cell death. As seen in Table 3, the cell death was rated from 0-4, with 0 representing no cell death and 4 being the strongest cell death response. P⁺ represents the presence of the pathogen specific proteases while P⁻ represents protease absence. The peptides displayed in Table 3 exhibited specific cell death response with ratings ranging from 3-4. See FIG. 4 for leaf illustration examples of this data.

TABLE 3
Cleavage sites exhibiting the greatest cell death response

| 6-mer sequence | Code | P+ | P− |
|---|---|---|---|
| HLYLYH | PBS1-S5 | 4 | 0 |
| LMFKMP | PBS1-S6 | 4 | 0 |
| WEGLMK | PBS1-S20 | 4 | 0 |
| FELMKA | PBS1-S21 | 4 | 0 |
| LMKLHN | PBS1-S23 | 3 | 0 |
| KHGLMR | PBS1-S24 | 4 | 0 |

### Example 6. Efficacy with Both Soy Rust and Soybean Cyst Nematode Cleavage Sites of Variable Length

In another example, a substrate protein could be constructed containing both a soy rust and a nematode cleavage site. The cleavage site can be 12-35 amino acids long. Cleavage of the cleavage site should result in increased resistance to both soy rust and nematode damage. Example sequence peptides comprising rust and soybean cleavage sites are shown at SEQ ID NOs: 98-145. These may include, for example, peptides comprising a combination of at least one soy rust cleavage site sequence (such as a cleavage sequence selected from SEQ ID NOs: 8-29) and at least one soybean cyst nematode cleavage site sequence (such as a cleavage sequence selected from SEQ ID NOs: 98-145). In some embodiments, the peptide is a concatenated peptide comprising a concatenation of at least one soy rust cleavage site sequence and at least one soybean cyst nematode cleavage site sequence.

Thus, in other embodiments, the disclosure is directed to substrate proteins containing both a soy rust and a nematode cleavage site. The cleavage site can be 12-35 amino acids long. Cleavage of the cleavage site results in increased resistance to both soy rust and nematode damage.

### Example 7. Using RIN4 Substrate Protein System to Replace the Cleavage Site

In another example, a RIN4 substrate protein can be used in place of PBS1 or another HRS protein to replace the cleavage site to engineer resistance to a target pathogen species.

1. A recombinant nucleic acid molecule comprising:
a nucleotide sequence that encodes a hypersensitive response substrate (HRS) protein of a plant pathogen-specific protease, wherein the HRS protein comprises a heterologous Basidiomycete-specific protease cleavage site,
wherein the HRS protein comprises:
a. a PBS1 polypeptide;
b. a PBS1 polypeptide as set forth in SEQ ID NO: 1; or
c. a polypeptide comprising an amino acid sequence having at least 90%, 95%, 96%, 97%, 98%, or 99% sequence identity to SEQ ID NO: 1, wherein said polypeptide retains PBS1 activity.

2. A recombinant nucleic acid molecule comprising:
a nucleotide sequence that encodes a hypersensitive response substrate (HRS) protein of a plant pathogen-specific protease, wherein the HRS protein comprises a heterologous Basidiomycete-specific protease cleavage site,
wherein the HRS protein comprises:
a. a RIN4 polypeptide;
b. a RIN4 polypeptide as set forth in SEQ ID NO: 148; or
c. a polypeptide comprising an amino acid sequence having at least 90%, 95%, 96%, 97%, 98%, or 99% sequence identity to SEQ ID NO: 148, wherein said polypeptide retains RIN4 activity.

3. The recombinant nucleic acid molecule of claim 1, wherein the heterologous Basidiomycete-specific cleavage site comprises a cleavage sequence selected from the group consisting of SEQ ID NO: 9, 17, 21, 24, 26, 27, 28, 29, or 52, or a sequence having at least 95% identity to SEQ ID NO: 9, 17, 21, 24, 26, 27, 28, 29, or 52.

4. (canceled)

5. The recombinant nucleic acid molecule of claim 3, wherein the heterologous Basidiomycete-specific cleavage site encodes at least one of:
a. an amino acid sequence having a number of amino acids in the range of 5 to 35 and comprising SEQ ID NO: 9 therein;
b. an amino acid sequence a having number of amino acids in the range of 5 to 35, and comprising SEQ ID NO: 17 therein;
c. an amino acid sequence having a number of amino acids in the range of 5 to 35, and comprising SEQ ID NO: 21 therein;
d. an amino acid sequence having a number of amino acids in the range of 5 to 35, and comprising SEQ ID NO: 24 therein;
e. an amino acid sequence having a number of amino acids in the range of 5 to 35, and comprising SEQ ID NO: 26 therein;
f. an amino acid sequence having a number of amino acids in the range of 5 to 35, and comprising SEQ ID NO: 27 therein;
g. an amino acid sequence having a number of amino acids in the range of 5 to 35, and comprising SEQ ID NO: 28 therein;
h. an amino acid sequence having a number of amino acids in the range of 5 to 35, and comprising SEQ ID NO: 29 therein; or
l. an amino acid sequence having a number of amino acids in the range of 5 to 35, and comprising SEQ ID NO: 52 therein.

6-8. (canceled)

9. The HRS protein of claim 1, wherein the HRS protein is derived from Arabidopsis, Glycine, Hordeum, or Triticum.

10. The recombinant nucleic acid molecule of claim 1, wherein the Basidiomycete-specific heterologous cleavage site is located between about amino acid position 238 to about amino acid position 248 in reference to any of SEQ ID NOs: 1, and 53-97.

11. The recombinant nucleic acid molecule of claim 1, wherein the heterologous Basidiomycete-specific protease is from a Phakopsora plant pathogen species and wherein expression of the recombinant nucleic acid molecule in a plant enhances the resistance of the plant to a disease caused by a Basidiomycete plant pathogen species.

12. The recombinant nucleic acid molecule of claim 11, wherein the Phakopsora plant pathogen species is Phakopsora pachyrhizi and the disease is Asian Soybean Rust.

13. The recombinant nucleic acid molecule of claim 1, wherein cleavage of the heterologous Basidiomycete-specific protease cleavage site activates a RPS5 resistance protein and triggers a hypersensitive cell death response, wherein the RPS5 resistance protein comprises SEQ ID NO: 3 or an amino acid sequence having at least 90% sequence identity to SEQ ID NO: 3 that retains RPS5 activity.

14-15. (canceled)

16. The recombinant nucleic acid molecule of claim 1, wherein the nucleotide sequence encoding the hypersensitive response substrate (HRS) protein is operably linked to a promoter active in a plant.

17. The recombinant nucleic acid molecule of claim 1, further comprising an expression cassette comprising a promoter active in a plant operably linked to an R-gene that is activated by the HRS protein following cleavage at the Basidiomycete-specific protease cleavage site.

18. A vector comprising the recombinant nucleic acid molecule of claim 1.

19. A recombinant protein comprising a modified hypersensitive response substrate (HRS) protein of a plant pathogen-specific protease, wherein the modified HRS protein comprises a heterologous Basidiomycete-specific protease cleavage site, and wherein the modified HRS protein is encoded by the recombinant nucleic acid molecule of claim 1.

20. A plant, plant cell, plant part or a seed comprising the recombinant nucleic acid molecule of claim 1.

21. The plant, plant cell, plant part or a seed of claim 20, wherein the plant, plant part or plant cell is a dicot.

22. The plant, plant part, plant cell or a seed of claim 21, wherein the dicot is a member of the genus Glycine.

23. A method of enhancing pathogen resistance in a plant from a disease caused upon infection by a Basidiomycete plant pathogen species, the method comprising:
expressing in the plant a nucleotide sequence that encodes a hypersensitive response substrate (HRS) protein of a plant pathogen-specific protease comprising a heterologous Basidiomycete-specific cleavage site, wherein cleavage of the heterologous Basidiomycete-specific cleavage site by the protease confers resistance to a disease caused by the Basidiomycete plant pathogen species,
wherein the heterologous Basidiomycete-specific cleavage site comprises a cleavage sequence selected from the group consisting of SEQ ID NO: 9, 17, 21, 24, 26, 27, 28, 29, or 52, or a sequence having at least 90% identity to SEQ ID NO: 9, 17, 21, 24, 26, 27, 28, 29 or 52; and
wherein the HRS protein comprises a PBS1 polypeptide as set forth in SEQ ID NO: 1, a RIN4 polypeptide as set forth in SEQ ID NO: 148, a polypeptide comprising an amino acid sequence having at least 90% sequence identity to SEQ ID NO: 1 and retaining PBS1 activity, or a polypeptide comprising an amino acid sequence having at least 90% sequence identity to SEQ ID NO: 148 and retaining RIN4 activity.

24. The method of claim 23, wherein said nucleotide sequence that encodes the HRS protein is introduced into the plant by transformation.

25. The method of claim 23, wherein said nucleotide sequence that encodes the HRS protein is introduced into the plant by genome modification.

26. The method of claim 23,
wherein the Basidiomycete plant pathogen species is Phakopsora pachyrhizi and the disease is Asian Soybean Rust (ASR); and
wherein cleavage of the HRS protein at the cleavage site by the protease confers improved resistance to Phakopsora pachyrhizi and ASR.

27. A recombinant nucleic acid molecule comprising:
a nucleotide sequence that encodes a hypersensitive response substrate (HRS) protein of a plant pathogen-specific protease, wherein the HRS protein has a heterologous Nematoda-specific protease cleavage site.

28. The recombinant nucleic acid molecule of claim 27, wherein the HRS protein comprises:
a. a PBS1 polypeptide;
b. a PBS1 polypeptide as set forth in SEQ ID NO: 1,
c. an amino acid sequence having at least 80%, 85%, 90%, 95%, 96%, 97%, 98%, or 99% sequence identity to SEQ ID NO: 1, wherein said polypeptide retains PBS1 activity;
d. a RIN4 polypeptide;
e. a RIN4 polypeptide as set forth in SEQ ID NO: 148; or
f. an amino acid sequence having at least 80% 85%, 95%, 96%, 97%, 98%, or 99% sequence identity to SEQ ID NO: 148, wherein said polypeptide retains RIN4 activity.

29. The recombinant nucleic acid molecule of claim 27 or 28, wherein the heterologous Nematoda-specific cleavage site comprises a cleavage sequence selected from the group consisting of SEQ ID NO: 34, 35, 48, 49, 50, or 51, or a sequence having at least 95% identity to SEQ ID NO: 34, 35, 48, 49, 50, or 51.

30-45. (canceled)

46. A recombinant protein comprising a modified hypersensitive response substrate (HRS) protein of a plant pathogen-specific protease, wherein the modified HRS protein comprises an amino acid sequence having a heterologous Nematoda-specific cleavage site, and wherein the modified HRS protein is encoded by the recombinant nucleic acid molecule according to claim 27.

47. A method of enhancing pathogen resistance in a plant from a disease caused from infection by a Nematoda plant pathogen species, the method comprising:
expressing in the plant a nucleotide sequence that encodes a hypersensitive response substrate (HRS) protein of a plant pathogen-specific protease comprising a heterologous Nematoda-specific cleavage site of claim 27, wherein cleavage of the heterologous Nematoda-specific cleavage site by the protease confers the plant with resistance to a disease caused by the Nematoda plant pathogen species.

48. The method of claim 47, wherein the Nematoda plant pathogen species is Heterodera glycines and the disease is Soybean Cyst Nematode (SCN);
wherein the heterologous Nematoda-specific cleavage site is a Heterodera-specific heterologous cleavage site comprising a cleavage sequence selected from the group consisting of SEQ NO: 34, 35, 48, 49, 50, or 51 or a sequence having at least 65% identity to SEQ ID NO: 34, 35, 48, 49, 50, or 51; and
wherein cleavage of the HRS protein at the cleavage site by the protease confers improved resistance to Heterodera glycines and SCN.

49-52. (canceled)

53. An isolated cleavage site peptide for insertion into an HRS protein to create engineered resistance, wherein the cleavage site peptide is selected from the group consisting of SEQ ID NOS: 8-52, and 98-145.

54. A recombinant protein comprising a modified hypersensitive response substrate (HRS) protein of a plant pathogen-specific protease, wherein the modified HRS protein comprises a heterologous Basidiomycete-specific protease cleavage site, and wherein the modified HRS protein is encoded by the recombinant nucleic acid molecule according to claim 2.

55. A plant, plant cell, plant part or a seed comprising the recombinant nucleic acid molecule according to claim 2.
